(19) United States
(12) Patent Application Publication
(10) Pub. No.: US 2024/0352143 A1
(43) Pub. Date: Oct. 24, 2024

(54) ANTI-CD73 ANTIBODIES AND USES THEREOF

(71) Applicant: Incyte Corporation, Wilmington, DE (US)

(72) Inventors: Horacio G. Nastri, West Chester, PA (US); Shaun M. Stewart, Chadds Ford, PA (US); Juan Carlos Almagro, Cambridge, MA (US); Jing Zhou, Boxborough, MA (US); Rebecca A. Buonpane, Wilmington, DE (US)

(21) Appl. No.: 18/668,914

(22) Filed: May 20, 2024

Related U.S. Application Data
(62) Division of application No. 17/138,279, filed on Dec. 30, 2020, now Pat. No. 12,018,089.
(60) Provisional application No. 62/956,847, filed on Jan. 3, 2020.

(51) Int. Cl. C07K 16/28; A61K 39/00; A61P 35/00

(52) U.S. Cl. CPC C07K 16/2896; A61P 35/00; A61K 2039/505; C07K 2317/24; C07K 2317/33; C07K 2317/34; C07K 2317/76; C07K 2317/92

(57) ABSTRACT

Anti-CD73 antibodies are disclosed. Also disclosed are related nucleic acids, vectors, cells, and pharmaceutical compositions. Methods of treating cancer with the anti-CD73 antibodies are also disclosed.

### RELATED APPLICATIONS

This application is a divisional of U.S. application Ser. No. 17/138,279, filed on Dec. 30, 2020, which claims the benefit of U.S. Provisional Application No. 62/956,847, filed Jan. 3, 2020, each of which is incorporated herein by reference in its entirety.

### SEQUENCE LISTING

This application contains a Sequence Listing that has been submitted electronically as an XML file named 20443-0644002_SL_ST26.xml. The XML file, created on Jan. 23, 2024, is 92,415 bytes in size. The material in the XML file is hereby incorporated by reference in its entirety.

### BACKGROUND

Cluster of differentiation 73 (CD73) is a glycosyl phosphatidyl inositol-(GPI-)linked membrane protein that catalyzes the conversion of extracellular adenosine monophosphate (AMP) to adenosine. It functions as a homodimer, and can be shed and is active as a soluble protein in circulation. In addition to its enzymatic function, CD73 also is a cellular adhesion molecule and plays a role in regulation of leukocyte trafficking. CD73 levels are known to be upregulated due to tissue injury or hypoxic conditions, and a number of solid tumors have elevated CD73 levels. Upregulation of CD73 within the tumor contributes to the adenosine-rich tumor microenvironment, which has numerous pro-tumor and immuno-suppressive effects. Thus, there is a need for therapeutics targeting CD73 in cancer.

### SUMMARY

In one aspect, the disclosure provides an antibody that binds to human CD73, wherein the antibody comprises a variable heavy (VH) domain comprising VH complementarity determining region (CDR)1, VH CDR2, and VH CDR3, wherein:
the VH CDR1 comprises the amino acid sequence GYTFTSYG (SEQ ID NO:1);
the VH CDR2 comprises the amino acid sequence IYPGSGNT (SEQ ID NO:2); and
the VH CDR3 comprises the amino acid sequence ARYDYLGSSYGFDY (SEQ ID NO:3); and
wherein the antibody comprises a variable light (VL) domain comprising VL CDR1, VL CDR2, and VL CDR3, wherein:
the VL CDR1 comprises the amino acid sequence QDVSTA (SEQ ID NO:4);
the VL CDR2 comprises the amino acid sequence SAS (SEQ ID NO:5); and
the VL CDR3 comprises the amino acid sequence QQHYNTPYT (SEQ ID NO:6).

In some embodiments of the first aspect, the VH domain comprises the amino acid sequence set forth in SEQ ID NO:22.

In some embodiments of the first aspect, the antibody comprises a heavy chain and wherein the heavy chain comprises the amino acid sequence set forth in SEQ ID NO:24.

In some embodiments of the first aspect, the VL domain comprises the amino acid sequence set forth in SEQ ID NO:23.

In some embodiments of the first aspect, the antibody comprises a light chain and wherein the light chain comprises the amino acid sequence set forth in SEQ ID NO:25.

In some embodiments of the first aspect, the VH domain is at least 80% identical to the amino acid sequence set forth in SEQ ID NO:22 and the VL domain is at least 80% identical to the amino acid sequence set forth in SEQ ID NO:23.

In some embodiments of the first aspect, the VH domain comprises the amino acid sequence set forth in SEQ ID NO:22 and the VL domain comprises the amino acid sequence set forth in SEQ ID NO:23.

In some embodiments of the first aspect, the antibody comprises a heavy chain and a light chain, and wherein the heavy chain comprises the amino acid sequence set forth in SEQ ID NO:24 and the light chain comprises the amino acid sequence set forth in SEQ ID NO:25.

In a second aspect, the disclosure provides an antibody that binds to human CD73, wherein the antibody binds to human CD73 at an epitope within amino acids 40-53 of SEQ ID NO:70.

In a third aspect, the disclosure provides an antibody that binds to human CD73 and competes for binding to human CD73 with an antibody that has a heavy chain comprising the amino acid sequence of SEQ ID NO:24 and a light chain comprising the amino acid sequence of SEQ ID NO:25.

In some embodiments of the first, second, and third aspects, the antibody is a humanized antibody.

In some embodiments of the first, second, and third aspects, the antibody is a bispecific antibody, single chain antibody, an Fab fragment, an F(ab′)2 fragment, an Fab′ fragment, an Fsc fragment, an Fv fragment, an scFv, an sc(Fv)2, or a diabody.

In a fourth aspect, the disclosure provides a nucleic acid or nucleic acids encoding the antibody of any one of the first through third aspects.

In a fifth aspect, the disclosure provides an expression vector or expression vectors comprising the foregoing nucleic acid or nucleic acids of the fourth aspect.

In a sixth aspect, the disclosure provides an isolated cell comprising the nucleic acid or nucleic acids of the fourth aspect or the expression vector or expression vectors of the fifth aspect.

In a seventh aspect, the disclosure provides an isolated cell comprising a first expression vector comprising a first nucleic acid encoding a first polypeptide comprising the VH domain of the antibody of any one of the first through third aspects operably linked to a promoter, and a second expression vector comprising a second nucleic acid encoding a second polypeptide comprising the VL domain of the antibody of any one of the first through third aspects operably linked to a promoter.

In an eighth aspect, the disclosure provides an isolated cell that produces the antibody of any one of the first through third aspects.

In a ninth aspect, the disclosure provides a method of making the antibody of any one of the first through third aspects, comprising culturing the cell of the sixth or seventh aspect and isolating the antibody.

In a tenth aspect, the disclosure provides a pharmaceutical composition comprising the antibody of any one of the first through third aspects and a pharmaceutically acceptable carrier.

In an eleventh aspect, the disclosure provides a method for treating a cancer in a human subject in need thereof, comprising administering to the human subject an effective amount of the antibody of any one of the first through third aspects.

In some embodiments of the eleventh aspect, the cancer has a high adenosine signature.

In some embodiments of the eleventh aspect, the cancer is head and neck cancer, colorectal cancer, lung cancer, melanoma, ovarian, bladder, liver cancer, or renal cell carcinoma.

In a twelfth aspect, the disclosure provides an antibody that binds to human CD73, wherein the antibody comprises a variable heavy (VH) domain comprising VH complementarity determining region (CDR)1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
wherein the antibody comprises a variable light (VL) domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39).

In some embodiments of the twelfth aspect,
the VH CDR1 comprises the amino acid sequence GFTFSSYD (SEQ ID NO:34);
the VH CDR2 comprises the amino acid sequence MSYDGSNK (SEQ ID NO:35);
the VH CDR3 comprises the amino acid sequence ATEIAAKGDY (SEQ ID NO:36);
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39). In some embodiments, the VH domain comprises the amino acid sequence set forth in SEQ ID NO:62. In some embodiments, the antibody comprises a heavy chain and wherein the heavy chain comprises the amino acid sequence set forth in SEQ ID NO:30. In some embodiments, the VL domain comprises the amino acid sequence set forth in SEQ ID NO:61. In some embodiments, the antibody comprises a light chain and wherein the light chain comprises the amino acid sequence set forth in SEQ ID NO:31. In some embodiments, the VH domain is at least 80% identical to the amino acid sequence set forth in SEQ ID NO:62 and the VL domain is at least 80% identical to the amino acid sequence set forth in SEQ ID NO:61. In some embodiments, the VH domain comprises the amino acid sequence set forth in SEQ ID NO:62 and the VL domain comprises the amino acid sequence set forth in SEQ ID NO:61.

In some embodiments, the antibody comprises a heavy chain and a light chain, and wherein the heavy chain comprises the amino acid sequence set forth in SEQ ID NO:30 and the light chain comprises the amino acid sequence set forth in SEQ ID NO:31.

In some embodiments of the twelfth aspect,
the VH CDR1 comprises the amino acid sequence GFTFSSYD (SEQ ID NO:34);
the VH CDR2 comprises the amino acid sequence MSYEGSNK (SEQ ID NO:40);
the VH CDR3 comprises the amino acid sequence ATEIAAKGDY (SEQ ID NO:36);
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39). In some embodiments, the VH domain comprises the amino acid sequence set forth in SEQ ID NO:63. In some embodiments, the antibody comprises a heavy chain and wherein the heavy chain comprises the amino acid sequence set forth in SEQ ID NO:33. In some embodiments, the VL domain comprises the amino acid sequence set forth in SEQ ID NO:61. In some embodiments, the antibody comprises a light chain and wherein the light chain comprises the amino acid sequence set forth in SEQ ID NO:31. In some embodiments, the VH domain is at least 80% identical to the amino acid sequence set forth in SEQ ID NO:63 and the VL domain is at least 80% identical to the amino acid sequence set forth in SEQ ID NO:61. In some embodiments, the VH domain comprises the amino acid sequence set forth in SEQ ID NO:63 and the VL domain comprises the amino acid sequence set forth in SEQ ID NO:61. In some embodiments, the antibody comprises a heavy chain and a light chain, and wherein the heavy chain comprises the amino acid sequence set forth in SEQ ID NO:33 and the light chain comprises the amino acid sequence set forth in SEQ ID NO:31.

In a thirteenth aspect, the disclosure provides an antibody that binds to human CD73, wherein the antibody binds to human CD73 at an epitope within amino acids 386-399 and 470-489 of SEQ ID NO:70.

In a fourteenth aspect, the disclosure provides an antibody that binds to human CD73 and competes for binding to human CD73 with an antibody that has a heavy chain comprising the amino acid sequence of SEQ ID NO:30 and a light chain comprising the amino acid sequence of SEQ ID NO:31.

In a fifteenth aspect, the disclosure provides an antibody that binds to human CD73 and competes for binding to human CD73 with an antibody that has a heavy chain comprising the amino acid sequence of SEQ ID NO:33 and a light chain comprising the amino acid sequence of SEQ ID NO:31.

In some embodiments of the twelfth through fifteenth aspects, the antibody is a humanized antibody.

In some embodiments of the twelfth through fifteenth aspects, the antibody is a bispecific antibody, single chain antibody, an Fab fragment, an F(ab′)2 fragment, an Fab′ fragment, an Fsc fragment, an Fv fragment, an scFv, an sc(Fv)2, or a diabody.

In a sixteenth aspect, the disclosure provides a nucleic acid or nucleic acids encoding the antibody thereof of any one of the twelfth through fifteenth aspects.

In a seventeenth aspect, the disclosure provides an expression vector or expression vectors comprising the nucleic acid or nucleic acids of the sixteenth aspect operably linked to a promoter.

In an eighteenth aspect, the disclosure provides an isolated cell comprising the nucleic acid or nucleic acids of the sixteenth aspect or the expression vector or expression vectors of the seventeenth aspect.

In a nineteenth aspect, the disclosure provides an isolated cell comprising a first expression vector comprising a first nucleic acid encoding a first polypeptide comprising the VH domain of the antibody of any one of the twelfth through fifteenth aspects operably linked to a promoter, and a second expression vector comprising a second nucleic acid encoding a second polypeptide comprising the VL domain of the antibody of any one of the twelfth through fifteenth aspects operably linked to a promoter.

In a twentieth aspect, the disclosure provides an isolated cell that produces the antibody of any one of the twelfth through fifteenth aspects.

In a twenty-first aspect, the disclosure provides a method of making the antibody of any one of the twelfth through fifteenth aspects, comprising culturing the cell of the eighteenth or nineteenth aspect and isolating the antibody.

In a twenty-second aspect, the disclosure provides a pharmaceutical composition comprising the antibody of any one of the twelfth through fifteenth aspects and a pharmaceutically acceptable carrier.

In a twenty-third aspect, the disclosure provides a method for treating a cancer in a human subject in need thereof, comprising administering to the human subject an effective amount of the antibody of any one of the twelfth through fifteenth aspects.

In some embodiments of the twenty-third aspect, the cancer has a high adenosine signature. In some embodiments of the twenty-third aspect, the cancer is head and neck cancer, colorectal cancer, lung cancer, melanoma, ovarian, bladder, liver cancer, or renal cell carcinoma.

### BRIEF DESCRIPTION OF THE DRAWINGS

FIG. 1A shows the heavy chain variable domain (VH) and light chain variable domain (VL) amino acid sequences for humanized CL25 antibodies CL_hu10-4, HzCL25, CL25_hu_10-6, and CL25_hu_11-4.

FIG. 1B shows the VH and VL amino acid sequences for humanized CL25 antibodies CL25_hu_11-5, CL25_hu_11-6, CL25_hu_8-4, and CL25_hu_8-5.

FIG. 1C shows the VH and VL amino acid sequences for humanized CL25 antibodies CL25_hu_8-6, CL25_hu_9-4, CL25_hu_9-5, and CL25_hu_9-6.

FIG. 1D shows an alignment of the VH for CL25 and humanized CL25 antibodies. CDRs according to the IMGT definition are underlined.

FIG. 1E shows an alignment of the VL for CL25 and humanized CL25 antibodies. CDRs according to the IMGT definition are underlined.

FIG. 2A is a graph depicting the cell binding (measured by geometric mean fluorescence intensity [GMFI]) for the indicated antibodies at the indicated concentrations on MDA-MB-231 cells.

FIG. 2B is a graph depicting the cell binding (measured by GMFI) for CL25, or isotype control (iso ctrl) at the indicated concentrations on A375 cells.

FIG. 3A is a graph depicting the cellular CD73 inhibition on A375 cells treated with the indicated antibodies or isotype control (iso ctrl) at the indicated concentrations.

FIG. 3B is a graph depicting the cellular CD73 inhibition on MDA-MB-231 cells treated with the indicated antibodies or isotype control (iso ctrl) at the indicated concentrations.

FIG. 3C is a graph depicting the cellular CD73 inhibition on MDA-MB-231 cells treated with the indicated antibodies or isotype control (iso ctrl) at the indicated concentrations.

FIG. 4 is a graph depicting inhibition of recombinant CD73 treated with the indicated antibodies or isotype control (iso ctrl) at the indicated concentrations.

FIGS. 5A-5N are each graphs depicting the percent CD4+ T cell proliferation in donor cells treated with the indicated antibody or isotype control at various concentrations.

FIG. 6 is a map of the crystal structure of human CD73 (4H2F.pdb) with the CL25 antibody epitope indicated in dark grey (with arrows).

FIG. 7 is a graph depicting surface CD73 levels after 24 hour incubation with the indicated antibody or isotype control (iso ctrl), or without treatment (NT), as measured with a directly conjugated non-competing antibody.

FIG. 8 is a graph depicting the mean tumor volume at the indicated days post tumor inoculation for mice administered isotype control (IgG control) or 1 mg/kg or 10 mg/kg of HzCL25.

FIG. 9 is a graph depicting the percent inhibition of CD73 in tumors harvested from mice treated administered isotype control (IgG control) or 1 mg/kg or 10 mg/kg of HzCL25.

FIG. 10A is a graph depicting the total surface CD73 levels for single cell suspensions of tumor cells only from mice treated with the indicated antibodies at the indicated doses, as determined by the mean fluorescence intensity (MFI) of non-competing antibody.

FIG. 10B is a graph depicting the receptor occupancy for single cell suspensions of tumor cells only from mice treated with the indicated antibodies at the indicated doses, as determined by the MFI of competing antibody.

FIG. 11A is a graph depicting the mean tumor volume at the indicated days post-inoculation with 10 mg/kg of isotype control (cx00376-001) or HzCL25. Triangles on the x-axis indicate days of drug administration.

FIG. 11B is a graph depicting the individual tumor volume through day 33 for the mice of FIG. 11A treated with isotype control (left) or HzCL25 (right).

FIG. 12 are graphs depicting the concentration of total and free soluble hCD73 in sera of mice treated with the indicated antibody or IgG isotype control. Total soluble hCD73 was detected with non-competing antibody (Antibody X, bottom) and free soluble hCD73 was detected with competing antibody (HzCL25, top).

FIG. 13A is a graph depicting the cell binding (GMFI) for antibody 3-F03 at the indicated concentrations on MDA-MB-231 cells.

FIG. 13B is a graph depicting the cell binding (measured by GMFI) for 3-F03 or isotype control (Iso ctrl) at the indicated concentrations on A375 cells.

FIG. 14A is a graph depicting the cellular CD73 inhibition on A375 cells treated with 3-F03 or isotype control (Iso ctrl) at the indicated concentrations.

FIG. 14B is a graph depicting the cellular CD73 inhibition on MDA-MB-231 cells treated with 3-F03 or isotype control (Iso ctrl) at the indicated concentrations.

FIG. 15 is a graph depicting inhibition of recombinant CD73 treated with 3-F03 or isotype control (Iso control) at the indicated concentrations.

FIGS. 16A-16D are each graphs depicting the percent CD4+ T cell proliferation in donor cells treated with the 3-F03 antibody or isotype control at various concentrations.

FIG. 17 is a map of the crystal structure of human CD73 (4H2F.pdb) with the 3-F03 antibody epitope indicated in dark grey (with arrows).

FIG. 18 is a graph depicting CD73 surface levels after 24 hour incubation with 3-F03, isotype control (iso ctrl), or not treated (NT) as measured with a directly conjugated non-competing antibody, CL43-Dy650.

FIG. 19A is a graph depicting the mean tumor volume at the indicated days post-inoculation with 10 mg/kg of isotype control (cx00376-001) or 3-F03. Triangles on x-axis indicate days of drug administration.

FIG. 19B is a graph depicting the individual tumor volume through day 33 for the mice of FIG. 19A treated with isotype control (left) or 3-F03 (right).

FIG. 20 are graphs depicting the concentration of total and free soluble hCD73 in sera of mice treated with 3-F03 or IgG isotype control. Total soluble hCD73 was detected with non-competing antibody (Antibody X, top) and free soluble hCD73 was detected with competing antibody (3-F03, bottom).

FIG. 21A-FIG. 21J show the VH and VL amino acid sequences of 3-F03 and exemplary 3-F03 variants.

FIG. 22 is a graph depicting cell binding (GMFI) for the indicated antibodies at the indicated concentrations on MDA-MB-231 cells.

FIG. 23 is a graph depicting the cellular CD73 inhibition on MDA-MB-231 cells treated with the indicated antibodies or isotype control at the indicated concentrations.

FIG. 24 shows exemplary amino acid sequences of a human IgG1 heavy chain CH1-hinge-CH2-CH3 with an N297A mutation (SEQ ID NO:73), a human IgG1 heavy chain CH1-hinge-CH2-CH3 with an N297A mutation with C-terminal lysine (SEQ ID NO:75), and a human kappa light chain constant region (SEQ ID NO:74).

FIG. 25A shows the DNA sequences encoding the HzCL25 heavy chain and light chain.

FIG. 25B shows the DNA sequence encoding the 3-F03_411 heavy chain.

FIG. 25C shows the DNA sequences encoding the 3-F03_411 and 3-F03_413 light chain and 3-F03_413 heavy chain.

FIG. 26 is a graph depicting the concentration of adenosine in tumors treated with isotype control antibody, HzCL25, or 3-F03.

FIG. 27 is a graph depicting inhibition of adenosine at the indicated concentrations of HzCL25 antibody (Ab) and AMP.

FIG. 28 is a graph depicting inhibition of adenosine at the indicated concentrations of 3-F03 antibody (Ab) and AMP.

FIG. 29 is a graph depicting inhibition of adenosine at the indicated concentrations of HzCL25 antibody (Ab) and AMP.

### DETAILED DESCRIPTION

Provided herein are anti-CD73 antibodies and related nucleic acids, expression vectors, cells, and pharmaceutical compositions. The anti-CD73 antibodies described herein are useful in the treatment or prevention of disorders such as cancer (e.g., head and neck cancer, colorectal cancer, lung cancer, melanoma, ovarian, bladder, liver cancer, or renal cell carcinoma).

CD73 CD73 (also known as “5′-nucleotidase” and “ecto-5′-nucleotidase”) is a dimeric enzyme (EC:3.1.3.5) that functions as a homodimer bound by a GPI linkage to the external face of the plasma membrane. CD73 can be shed and is active as a soluble protein in circulation. CD73 catalyzes the conversion of extracellular AMP to adenosine. CD73 enzymatic activity requires substrate binding in the open CD73 conformation. After the substrate binding, CD73 goes through a large conformational change from open to closed conformation to convert AMP to adenosine (see, e.g., Knapp et al., 2012, Structure, 20(12):2161-73). CD73 also functions as a cellular adhesion molecule and plays a role in regulation of leukocyte trafficking.

CD73 enzymatic activity plays a role in the promotion and metastasis of cancer (see, e.g., Stagg and Smyth, 2010, Oncogene, 29:5346-5358; Salmi and Jalkanen, 2012, OncoImmunology, 1:247-248, 2012; Stagg, 2012, OncoImmunology, 1:217-218; Zhang, 2012, OncoImmunology, 167-70). Overexpression of CD73 in cancer cells impairs adaptive antitumor immune responses, enhancing tumor growth and metastasis (see, e.g., Niemela et al., 2004, J. Immunol., 172:1646-1653; Sadej et al., 2006, Nucleosides Nucleotides Nucleic Acids, 25:1119-1123; Braganhol et al., 2007, Biochim. Biophys. Acta., 1770:1352-1359; Zhang, 2010, Cancer Res., 70:6407-6411; Zhang, 2012, OncoImmunology, 1:67-70).

An exemplary amino acid sequence of the mature human CD73 protein (amino acids 27-549 of GenBank Accession No. NP_002517) is:

WELTILHTNDVHSRLEQTSEDSSKCVNASRCMGGVARLFTKVQQIRRAEPNVLLLDAGDQYQ GTIWFTVYKGAEVAHFMNALRYDAMALGNHEFDNGVEGLIEPLLKEAKFPILSANIKAKGPL ASQISGLYLPYKVLPVGDEVVGIVGYTSKETPFLSNPGTNLVFEDEITALQPEVDKLKTLNV NKIIALGHSGFEMDKLIAQKVRGVDVVVGGHSNTFLYTGNPPSKEVPAGKYPFIVTSDDGRK VPVVQAYAFGKYLGYLKIEFDERGNVISSHGNPILLNSSIPEDPSIKADINKWRIKLDNYST QELGKTIVYLDGSSQSCRFRECNMGNLICDAMINNNLRHTDEMFWNHVSMCILNGGGIRSPI DERNNGTITWENLAAVLPFGGTFDLVQLKGSTLKKAFEHSVHRYGQSTGEFLQVGGIHVVYD LSRKPGDRVVKLDVLCTKCRVPSYDPLKMDEVYKVILPNFLANGGDGFQMIKDELLRHDSGD QDINVVSTYISKMKVIYPAVEGRIKFS (SEQ ID NO:70).

An exemplary amino acid sequence of the mature murine CD73 protein (amino acids 29-551 of GenBank Accession No. NP_035981) is:

(SEQ ID NO: 71)
WELTILHTNDVHSRLEQTSDDSTKCLNASLCVGGVARLFTKVQQIRKEEP
NVLFLDAGDQYQGTIWFTVYKGLEVAHFMNILGYDAMALGNHEFDNGVEG
LIDPLLRNVKFPILSANIKARGPLAHQISGLFLPSKVLSVGGEVVGIVGY
TSKETPFLSNPGINLVFEDEISALQPEVDKLKTLNVNKIIALGHSGFEMD
KLIAQKVRGVDIVVGGHSNTFLYTGNPPSKEVPAGKYPFIVTADDGRQVP
VVQAYAFGKYLGYLKVEFDDKGNVITSYGNPILLNSSIPEDATIKADINQ
WRIKLDNYSTQELGRTIVYLDGSTQTCRFRECNMGNLICDAMINNNLRHP
DEMFWNHVSMCIVNGGGIRSPIDEKNNGTITWENLAAVLPFGGTFDLVQL
KGSTLKKAFEHSVHRYGQSTGEFLQVGGIHVVYDINRKPWNRVVQLEVLC
TKCRVPIYEPLEMDKVYKVTLPSYLANGGDGFQMIKDELLKHDSGDQDIS
VVSEYISKMKVVYPAVEGRIKFS

An exemplary amino acid sequence of the mature cynomolgus CD73 protein is:

(SEQ ID NO: 72)
WELTILHTNDVHSRLEQTSEDSSKCVNASRCMGGVARLFTKVQQIRRAEP
NVLLLDAGDQYQGTIWFTVYKGAEVAHFMNALRYDAMALGNHEFDNGVEG
LIEPLLKEAKFPILSANIKAKGPLASQISGLYLPYKVLPVGDEVVGIVGY
TSKETPFLSNPGTNLVFEDEITALQPEVDKLKTLNVNKIIALGHSGFETD
KLIAQKVRGVDVVVGGHSNTFLYTGNPPSKEVPAGKYPFIVTSDDGRKVP
VVQAYAFGKYLGYLKIEFDERGNVISSHGNPILLNSSIPEDPSIKADINK
WRIKLDNYSTQELGKTIVYLDGSSQSCRFRECNMGNLICDAMINNNLRHA
DEMFWNHVSMCILNGGGIRSPIDERNNGTITWENLAAVLPFGGTFDLVQL
KGSTLKKAFEHSVHRYGQSTGEFLQVGGIHVVYDLSRKPGDRVVKLDVLC
TKCRVPSYDPLKMDEIYKVILPNFLANGGDGFQMIKDELLRHDSGDQDIN
VVSTYISKMKVIYPAVEGRIKFS

### Anti-CD73 Antibodies

This disclosure provides anti-CD73 antibodies that are useful in treating cancer. These antibodies can bind human CD73.

In some instances, these antibodies bind human CD73 and cynomolgus CD73. In some instances, these antibodies bind human CD73 and cynomolgus CD73 and do not bind murine CD73. Such anti-CD73 antibodies include the sequences of an anti-CD73 monoclonal antibody, CL25, and a humanized version thereof, HzCL25, which humanized version thereof binds with high affinity to both human and cynomolgus CD73 (e.g., less than approximately 0.5 nM in the open conformations for both human and cynomolgus CD73), and has undetectable binding to mouse CD73.

In some instances, these antibodies bind human CD73, cynomolgus CD73, and murine CD73. Such anti-CD73 antibodies includes the sequences of a human anti-CD73 monoclonal antibody, 3-F03, which binds with high affinity to the open conformation of each of human, cynomolgus, and murine CD73 (e.g., approximately 0.37 nM for human CD73, approximately 0.734 nM for cynomolgus CD73, and approximately 1.66 nM for murine CD73). Such anti-CD73 antibodies have undetectable binding to the closed conformation of each of human, cynomolgus, and murine CD73, in the presence of an excess of a non-cleavable CD73 ligand, APCP.

### Antibody HzCL25

Antibody HzCL25 is a humanized IgG1/kappa monoclonal antibody with alanine at position Asparagine-297 (N297, according to EU numbering) of the heavy chain constant region to reduce effector function. It specifically binds human and cynomolgus CD73 with high affinity (K_{D}≤0.5 nM) and has low effector functionality.

HzCL25 was constructed from a chimeric version of the CL25 antibody. The CL25 murine heavy chain variable domain (VH) and light chain variable domain (VL) were obtained from a mouse immunized with recombinant human CD73 (SEQ ID NO:70) comprising a HIS-tag. Antibody sequences of the B cells were determined and the murine heavy chain variable domain (VH) (SEQ ID NO:26) and light chain variable domain (VL) (SEQ ID NO:27) were expressed as chimeras with human IgG1 Fc (heavy chain constant region comprising the amino acid sequence of SEQ ID NO:73 and kappa light chain constant region comprising the amino acid sequence of SEQ ID NO:74). Table 1, below, shows the amino acid sequences of the CL25 complementarity determining regions (CDRs) according to IMGT, Chothia, AbM, Kabat, and Contact numbering. Table 1, below, also shows the amino acid sequences of the CL25 mature VH and VL.

TABLE 1
CL25 CDRs, VH, and VL

| | IMGT | Chothia | AbM | Kabat | Contact |
|---|---|---|---|---|---|
| VH | GYTFTSYG | GYTFTSY | GYTFTSYGLS | SYGLS | TSYGLS |
| CDR1 | (SEQ ID NO: 1) | (SEQ ID NO: 7) | (SEQ ID NO: 12) | (SEQ ID NO: 14) | (SEQ ID NO: 16) |
| VH | IYPGSGNT | YPGSGN | EIYPGSGNTY | EIYPGSGNTYY | WIGEIYPGSGN |
| CDR2 | (SEQ ID NO: 2) | (SEQ ID NO: 8) | (SEQ ID NO: 13) | NEKFKG | TY |
| | | | | (SEQ ID NO: 15) | (SEQ ID NO: 28) |
| VH | ARYDYLGSSY | YDYLGSSYGFD | YDYLGSSYGFD | YDYLGSSYGFD | ARYDYLGSSY |
| CDR3 | GFDY | Y | Y | Y | GFD |
| | (SEQ ID NO: 3) | (SEQ ID NO: 9) | (SEQ ID NO: 9) | (SEQ ID NO: 9) | (SEQ ID NO: 18) |
| VL | QDVSTA | KASQDVSTAV | KASQDVSTAV | KASQDVSTAV | STAVAWY |
| CDR1 | (SEQ ID NO: 4) | A | A | A | (SEQ ID NO: 19) |
| | | (SEQ ID NO: 10) | (SEQ ID NO: 10) | (SEQ ID NO: 10) | |
| VL | SAS | SASYRYN | SASYRYN | SASYRYN | LLIYSASYRY |
| CDR2 | (SEQ ID NO: 5) | (SEQ ID NO: 29) | (SEQ ID NO: 29) | (SEQ ID NO: 29) | (SEQ ID NO: 20) |
| VL | QQHYNTPYT | QQHYNTPYT | QQHYNTPYT | QQHYNTPYT | QQHYNTPY |
| CDR3 | (SEQ ID NO: 6) | (SEQ ID NO: 6) | (SEQ ID NO: 6) | (SEQ ID NO: 6) | (SEQ ID NO: 21) |

| | |
|---|---|
| VH | QVQLQQSGAELARPGASVKLSCRASGYTFTSYGLSWVKQRTGQGLEWIGEIYPGSGNTYYNE |
| | KFKGKATLTADKSSSTAYMELRSLTSEDSAVYFCARYDYLGSSYGFDYWGQGTTLTVSS |
| | (SEQ ID NO: 26) |
| VL | DIVMTQSHKFMSTSVGDRVSITCKASQDVSTAVAWYQQKPGQSPKLLIYSASYRYNGVPDRF |
| | TGSGSGTDFTFTISSVQAEDLAVYYCQQHYNTPYTFGGGTKLEIK (SEQ ID NO: 27) |

To construct HzCL25, the CL25 VH and VL sequences were aligned to a database of human VH and VK genes. The CDRs (Table 1) from the murine CL25 antibody were grafted into human VH and VK genes.

Table 2, below, shows the amino acid sequences of the HzCL25 CDRs according to IMGT, Chothia, AbM, Kabat, and Contact numbering. Table 2, below, also shows the amino acid sequences of the HzCL25 mature VH, VL, heavy chain, and light chain.

TABLE 2
Amino acid sequences of HzCL25 CDRs, VH, VL, heavy chain, and light chain

| | IMGT | Chothia | AbM | Kabat | Contact |
|---|---|---|---|---|---|
| VH | GYTFTSYG | GYTFTSY | GYTFTSYGLS | SYGLS | TSYGLS |
| CDR1 | (SEQ ID NO: 1) | (SEQ ID NO: 7) | (SEQ ID NO: 12) | (SEQ ID NO: 14) | (SEQ ID NO: 16) |
| VH | IYPGSGNT | YPGSGN | EIYPGSGNTY | EIYPGSGNTYY | WMGEIYPGSG |
| CDR2 | (SEQ ID NO: 2) | (SEQ ID NO: 8) | (SEQ ID NO: 13) | NEKFKG | NTY |
| | | | | (SEQ ID NO: 15) | (SEQ ID NO: 17) |
| VH | ARYDYLGSSY | YDYLGSSYGFD | YDYLGSSYGFD | YDYLGSSYGFD | ARYDYLGSSY |
| CDR3 | GFDY | Y | Y | Y | GFD |
| | (SEQ ID NO: 3) | (SEQ ID NO: 9) | (SEQ ID NO: 9) | (SEQ ID NO: 9) | (SEQ ID NO: 18) |
| VL | QDVSTA | KASQDVSTAV | KASQDVSTAV | KASQDVSTAV | STAVAWY |
| CDR1 | (SEQ ID NO: 4) | A | A | A | (SEQ ID NO: 19) |
| | | (SEQ ID NO: 10) | (SEQ ID NO: 10) | (SEQ ID NO: 10) | |
| VL | SAS | SASYRYS | SASYRYS | SASYRYS | LLIYSASYRY |
| CDR2 | (SEQ ID NO: 5) | (SEQ ID NO: 11) | (SEQ ID NO: 11) | (SEQ ID NO: 11) | (SEQ ID NO: 20) |
| VL | QQHYNTPYT | QQHYNTPYT | QQHYNTPYT | QQHYNTPYT | QQHYNTPY |
| CDR3 | (SEQ ID NO: 6) | (SEQ ID NO: 6) | (SEQ ID NO: 6) | (SEQ ID NO: 6) | (SEQ ID NO: 21) |

| | |
|---|---|
| VH | EVQLVQSGAEVKKPGESLKISCKGSGYTFTSYGLSWVRQMPGKGLEWMGEIYPGSGNTYYN |
| | EKFKGQVTISADKSISTAYLQWSSLKASDTAMYYCARYDYLGSSYGFDYWGAGTTVTVSS |
| | (SEQ ID NO: 22) |
| VL | DIVMTQSPDSLAVSLGERATINCKASQDVSTAVAWYQQKPGQPPKLLIYSASYRYSGVPDRFS |
| | GSGSGTDFTLTISSLQAEDVAVYYCQQHYNTPYTFGGGTKLEIK (SEQ ID NO: 23) |
| Heavy | EVQLVQSGAEVKKPGESLKISCKGSGYTFTSYGLSWVRQMPGKGLEWMGEIYPGSGNTYYN |
| Chain | EKFKGQVTISADKSISTAYLQWSSLKASDTAMYYCARYDYLGSSYGFDYWGAGTTVTVSSA |
| | STKGPSVFPLAPSSKSTSGGTAALGCLVKDYFPEPVTVSWNSGALTSGVHTFPAVLQSSGLYS |
| | LSSVVTVPSSSLGTQTYICNVNHKPSNTKVDKRVEPKSCDKTHTCPPCPAPELLGGPSVFLFPP |
| | KPKDTLMISRTPEVTCVVVDVSHEDPEVKFNWYVDGVEVHNAKTKPREEQYASTYRVVSVL |
| | TVLHQDWLNGKEYKCKVSNKALPAPIEKTISKAKGQPREPQVYTLPPSREEMTKNQVSLTCL |
| | VKGFYPSDIAVEWESNGQPENNYKTTPPVLDSDGSFFLYSKLTVDKSRWQQGNVFSCSVMHE |
| | ALHNHYTQKSLSLSPG (SEQ ID NO: 24) |
| Light | DIVMTQSPDSLAVSLGERATINCKASQDVSTAVAWYQQKPGQPPKLLIYSASYRYSGVPDRFS |
| Chain | GSGSGTDFTLTISSLQAEDVAVYYCQQHYNTPYTFGGGTKLEIKRTVAAPSVFIFPPSDEQLKS |
| | GTASVVCLLNNFYPREAKVQWKVDNALQSGNSQESVTEQDSKDSTYSLSSTLTLSKADYEK |
| | HKVYACEVTHQGLSSPVTKSFNRGEC (SEQ ID NO: 25) |

The anti-CD73 antibodies can encompass the VH CDR1, VH CDR2, and VH CDR3 and the VL CDR1, VL CDR2, and VL CDR3 of HzCL25 or CL25. In some instances, the anti-CD73 antibody comprises a VH comprising VH CDR1, VH CDR2, and VH CDR3 of HzCL25 (see Table 2). In some instances, the anti-CD73 antibody comprises a VL comprising VL CDR1, VL CDR2, and VL CDR3 of HzCL25 (see Table 2). In some instances, the anti-CD73 antibody comprises a VH comprising VH CDR1, VH CDR2, and VH CDR3 of HzCL25 (see Table 2) and a VL comprising VL CDR1, VL CDR2, and VL CDR3 of HzCL25 (see Table 2). In some instances, the anti-CD73 antibody comprises a VH comprising VH CDR1, VH CDR2, and VH CDR3 of CL25 (see Table 1). In some instances, the anti-CD73 antibody comprises a VL comprising VL CDR1, VL CDR2, and VL CDR3 of CL25 (see Table 1). In some instances, the anti-CD73 antibody comprises a VH comprising VH CDR1, VH CDR2, and VH CDR3 of CL25 (see Table 1) and a VL comprising VL CDR1, VL CDR2, and VL CDR3 of CL25 (see Table 1). In some instances, the anti-CD73 antibodies can have, e.g., 1, 2, or 3 substitutions within one or more (i.e., 1, 2, 3, 4, 5, or 6) of the six CDRs of HzCL25 or CL25. In some instances, the antibodies (i) inhibit cellular CD73 (e.g., at least 10%; at least 20%, at least 30%, at least 40%, at least 50%, at least 60%, at least 70%, at least 80%, at least 90%, at least 95%, at least 97%, at least 99%, or 100% reduction in cellular CD73 activity as compared to an isotype control as determined by, e.g., the assay described in Example 3); and/or (ii) inhibit soluble CD73 (e.g., at least 10%; at least 20%, at least 30%, at least 40%, at least 50%, at least 60%, at least 70%, at least 80%, at least 90%, at least 95%, at least 97%, at least 99%, or 100% reduction in soluble CD73 activity as compared to an isotype control as determined by, e.g., the assay described in Example 4); and/or (iii) bind human or cynomolgus monkey CD73 in the open conformation with high affinity (e.g., K_{D}≤0.5 nM) but do not significantly bind CD73 in the open conformation from mice (e.g., as determined by the binding assay described in Example 6); and/or (iv) bind human or cynomolgus monkey CD73 in the closed conformation with high affinity (e.g., K_{D}≤0.5 nM) but do not significantly bind CD73 in the closed conformation from mice; and/or (v) bind to an epitope within amino acids 40-53 of SEQ ID NO:70 (i.e., within TKVQQIRRAEPNVL (SEQ ID NO:76)) (e.g., as determined by the binding assay described in Example 6); and/or (vi) reduce AMP-mediated suppression of T cell proliferation (e.g., at least 10%; at least 20%, at least 30%, at least 40%, at least 50%, at least 60%, at least 70%, at least 80%, at least 90%, at least 95%, at least 97%, at least 99%, or 100% reduction in T cell proliferation as compared to an isotype control as determined by, e.g., the assay described in Example 5); and/or (vii) decreases levels of cell surface CD73 (e.g., on cancer cells, e.g., on melanoma cancer cells, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control as determined by, e.g., the assay described in Example 8); and/or (viii) reduce tumor growth (e.g., melanoma tumors, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control as determined by, e.g., the assay described in Example 9); and/or (ix) reduce free surface CD73 on cells (e.g., cancer cells, e.g., melanoma cancer cancers, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control as determined by, e.g., the assay described in Example 11).

The anti-CD73 antibodies can comprise the VH CDR1, VH CDR2, and VH CDR3 of HzCL25 or CL25 according to the IMGT definition, or an alternate CDR definition such as, but not limited to, the Kabat definition, the Chothia definition, the AbM CDR definition, or the contact definition. These anti-CD73 antibodies may include zero, one, two, or three substitutions in VH CDR1 and/or VH CDR2 and/or VH CDR3 of HzCL25 or CL25. In some embodiments, the anti-CD73 antibodies further comprise the VL CDR1, VL CDR2, and VL CDR3 of HzCL25 or CL25 according to the IMGT definition, or an alternate CDR definition such as, but not limited to, the Kabat definition, the Chothia definition, the AbM CDR definition, or the contact definition. These anti-CD73 antibodies may include zero, one, two, or three substitutions in VL CDR1 and/or VL CDR2 and/or VL CDR3 of HzCL25 or CL25. In some instances, the anti-CD73 antibody comprises a VH comprising the VH CDR1, VH CDR2, and VH CDR3 set forth in SEQ ID NOs: 1, 2, and 3, respectively, and a VL comprising the VL CDR1, VL CDR2, and VL CDR3 set forth in SEQ ID NOs: 4, 5, and 6, respectively. In some instances, the anti-CD73 antibody comprises a VH comprising the VH CDR1, VH CDR2, and VH CDR3 set forth in SEQ ID NOs: 7, 8, and 9, respectively, and a VL comprising the VL CDR1, VL CDR2, and VL CDR3 set forth in SEQ ID NOs: 10, 11, and 6, respectively. In some instances, the anti-CD73 antibody comprises a VH comprising the VH CDR1, VH CDR2, and VH CDR3 set forth in SEQ ID NOs: 12, 13, and 9, respectively, and a VL comprising the VL CDR1, VL CDR2, and VL CDR3 set forth in SEQ ID NOs: 10, 11, and 6, respectively. In some instances, the anti-CD73 antibody comprises a VH comprising the VH CDR1, VH CDR2, and VH CDR3 set forth in SEQ ID NOs: 14, 15, and 9, respectively, and a VL comprising the VL CDR1, VL CDR2, and VL CDR3 set forth in SEQ ID NOs: 10, 11, and 6, respectively. In some instances, the anti-CD73 antibody comprises a VH comprising the VH CDR1, VH CDR2, and VH CDR3 set forth in SEQ ID NOs: 16, 17, and 18, respectively, and a VL comprising the VL CDR1, VL CDR2, and VL CDR3 set forth in SEQ ID NOs: 19, 20, and 21, respectively. In some instances, the anti-CD73 antibody comprises a VH comprising the VH CDR1, VH CDR2, and VH CDR3 set forth in SEQ ID NOs: 7, 8, and 9, respectively, and a VL comprising the VL CDR1, VL CDR2, and VL CDR3 set forth in SEQ ID NOs: 10, 29, and 6, respectively. In some instances, the anti-CD73 antibody comprises a VH comprising the VH CDR1, VH CDR2, and VH CDR3 set forth in SEQ ID NOs: 12, 13, and 9, respectively, and a VL comprising the VL CDR1, VL CDR2, and VL CDR3 set forth in SEQ ID NOs: 10, 29, and 6, respectively. In some instances, the anti-CD73 antibody comprises a VH comprising the VH CDR1, VH CDR2, and VH CDR3 set forth in SEQ ID NOs: 14, 15, and 9, respectively, and a VL comprising the VL CDR1, VL CDR2, and VL CDR3 set forth in SEQ ID NOs: 10, 29, and 6, respectively. In some instances, the anti-CD73 antibody comprises a VH comprising the VH CDR1, VH CDR2, and VH CDR3 set forth in SEQ ID NOs: 16, 28, and 18, respectively, and a VL comprising the VL CDR1, VL CDR2, and VL CDR3 set forth in SEQ ID NOs: 19, 20, and 21, respectively. In some instances these antibodies (i) inhibit cellular CD73 (e.g., at least 10%; at least 20%, at least 30%, at least 40%, at least 50%, at least 60%, at least 70%, at least 80%, at least 90%, at least 95%, at least 97%, at least 99%, or 100% reduction in cellular CD73 activity as compared to an isotype control as determined by, e.g., the assay described in Example 3); and/or (ii) inhibit soluble CD73 (e.g., at least 10%; at least 20%, at least 30%, at least 40%, at least 50%, at least 60%, at least 70%, at least 80%, at least 90%, at least 95%, at least 97%, at least 99%, or 100% reduction in soluble CD73 activity as compared to an isotype control as determined by, e.g., the assay described in Example 4); and/or (iii) bind human or cynomolgus monkey CD73 in the open conformation with high affinity (e.g., K_{D}≤0.5 nM) but do not significantly bind CD73 in the open conformation from mice (e.g., as determined by the binding assay described in Example 6); and/or (iv) bind human or cynomolgus monkey CD73 in the closed conformation with high affinity (e.g., K_{D}≤0.5 nM) but do not significantly bind CD73 in the closed conformation from mice; and/or (v) bind to an epitope within amino acids 40-53 of SEQ ID NO:70 (i.e., within TKVQQIRRAEPNVL (SEQ ID NO:76)) (e.g., as determined by the binding assay described in Example 6); and/or (vi) reduce AMP-mediated suppression of T cell proliferation (e.g., at least 10%; at least 20%, at least 30%, at least 40%, at least 50%, at least 60%, at least 70%, at least 80%, at least 90%, at least 95%, at least 97%, at least 99%, or 100% reduction in T cell proliferation as compared to an isotype control as determined by, e.g., the assay described in Example 5); and/or (vii) decreases levels of cell surface CD73 (e.g., on cancer cells, e.g., on melanoma cancer cells, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control as determined by, e.g., the assay described in Example 8); and/or (viii) reduce tumor growth (e.g., melanoma tumors, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control as determined by, e.g., the assay described in Example 9); and/or (ix) reduce free surface CD73 on cells (e.g., cancer cells, e.g., melanoma cancer cancers, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control as determined by, e.g., the assay described in Example 11).

In certain embodiments, the anti-CD73 antibodies comprise an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:22, 26, and 82-84. In certain embodiments, the anti-CD73 antibodies comprise a VH comprising the VH CDR1, VH CDR2, and VH CDR3 of HzCL25 (see Table 1, e.g., according to the IMGT definition, i.e., the amino acid sequences set forth in SEQ ID NOs: 1-3, respectively), wherein the VH comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:22, 26, and 82-84. In some embodiments, the anti-CD73 antibodies comprise an amino acid sequence having one or more (e.g., 1, 2, 3, 4, 5, 6, 7, 8, 9, 10) amino acid substitutions, additions, and/or deletions relative to the amino acid sequence set forth in SEQ ID NO:24. In some embodiments, the anti-CD73 antibodies comprise a heavy chain comprising the VH CDR1, VH CDR2, and VH CDR3 of HzCL25 (see Table 1, e.g., according to the IMGT definition, i.e., the amino acid sequences set forth in SEQ ID NOs: 1-3, respectively), wherein the heavy chain comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO:24. In certain embodiments, the anti-CD73 antibodies comprise an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:23, 27, 80, and 81. In certain embodiments, the anti-CD73 antibodies comprise a VL comprising the VL CDR1, VL CDR2, and VL CDR3 of HzCL25 (see Table 1, e.g., according to the IMGT definition, i.e., the amino acid sequences set forth in SEQ ID NOs: 4-6, respectively), wherein the VL comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:23, 27, 80, and 81. In some embodiments, the anti-CD73 antibodies comprise an amino acid sequence having one or more (e.g., 1, 2, 3, 4, 5, 6, 7, 8, 9, 10) amino acid substitutions, additions, and/or deletions relative to the amino acid sequence set forth in SEQ ID NO:25. In some embodiments, the anti-CD73 antibodies comprise a light chain comprising the VL CDR1, VL CDR2, and VL CDR3 of HzCL25 (see Table 1, e.g., according to the IMGT definition, i.e., the amino acid sequences set forth in SEQ ID NOs: 4-6, respectively), wherein the light chain comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO:25. In certain embodiments, the anti-CD73 antibodies comprise: (i) an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:22, 26, and 82-84; and (ii) an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:23, 27, 80, and 81. In certain embodiments, the anti-CD73 antibodies comprise: (i) a VH comprising the VH CDR1, VH CDR2, and VH CDR3 of HzCL25 (see Table 1, e.g., according to the IMGT definition, i.e., the amino acid sequences set forth in SEQ ID NOs: 1-3, respectively), wherein the VH comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:22, 26, and 82-84; and (ii) a VL comprising the VL CDR1, VL CDR2, and VL CDR3 of HzCL25 (see Table 1, e.g., according to the IMGT definition, i.e., the amino acid sequences set forth in SEQ ID NOs: 4-6, respectively), wherein the VL comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:23, 27, 80, and 81. In some embodiments, the anti-CD73 antibodies comprise: (i) an amino acid sequence having one or more (e.g., 1, 2, 3, 4, 5, 6, 7, 8, 9, 10) amino acid substitutions, additions, and/or deletions relative to the amino acid sequence set forth in SEQ ID NOs:24; and (ii) an amino acid sequence having one or more (e.g., 1, 2, 3, 4, 5, 6, 7, 8, 9, 10) amino acid substitutions, additions, and/or deletions relative to the amino acid sequence set forth in SEQ ID NOs:25. In some embodiments, the anti-CD73 antibodies comprise: (i) a heavy chain comprising the VH CDR1, VH CDR2, and VH CDR3 of HzCL25 (see Table 1, e.g., according to the IMGT definition, i.e., the amino acid sequences set forth in SEQ ID NOs: 1-3, respectively), wherein the heavy chain comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO:24; and (ii) a light chain comprising the VL CDR1, VL CDR2, and VL CDR3 of HzCL25 (see Table 1, e.g., according to the IMGT definition, i.e., the amino acid sequences set forth in SEQ ID NOs: 4-6, respectively), wherein the light chain comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO:25.

In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH set forth in any one of SEQ ID NOs: 22, 26, and 82-84. In certain embodiments, the anti-CD73 antibodies comprise a VH comprising the VH CDR1, VH CDR2, and VH CDR3 of HzCL25 (see Table 1, e.g., according to the IMGT definition, i.e., the amino acid sequences set forth in SEQ ID NOs: 1-3, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH set forth in any one of SEQ ID NOs: 22, 26, and 82-84. In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL set forth in any one of SEQ ID NOs: 23, 27, 80, and 81. In certain embodiments, the anti-CD73 antibodies comprise a VL comprising the VL CDR1, VL CDR2, and VL CDR3 of HzCL25 (see Table 1, e.g., according to the IMGT definition, i.e., the amino acid sequences set forth in SEQ ID NOs: 4-6, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL set forth in any one of SEQ ID NOs: 23, 27, 80, and 81. In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH set forth in any one of SEQ ID NOs: 22, 26, and 82-84 and an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL set forth in SEQ ID NO: 23, 27, 80, and 81. In certain embodiments, the anti-CD73 antibodies comprise: (i) a VH comprising the VH CDR1, VH CDR2, and VH CDR3 of HzCL25 (see Table 1, e.g., according to the IMGT definition, i.e., the amino acid sequences set forth in SEQ ID NOs: 1-3, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH set forth in any one of SEQ ID NOs: 22, 26, and 82-84, and (ii) a VL comprising the VL CDR1, VL CDR2, and VL CDR3 of HzCL25 (see Table 1, e.g., according to the IMGT definition, i.e., the amino acid sequences set forth in SEQ ID NOs: 4-6, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL set forth in any one of SEQ ID NOs: 23, 27, 80, and 81.

In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of HzCL25 (i.e., the amino acid sequence set forth in SEQ ID NO:22). In certain embodiments, the anti-CD73 antibodies comprise a VH comprising the VH CDR1, VH CDR2, and VH CDR3 of HzCL25 (see Table 1, e.g., according to the IMGT definition, i.e., the amino acid sequences set forth in SEQ ID NOs: 1-3, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of HzCL25 (i.e., the amino acid sequence set forth in SEQ ID NO:22). In certain embodiments, the anti-CD73 antibodies comprise a VH comprising the amino acid sequence set forth in SEQ ID NO:22. In some embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of HzCL25 (i.e., the amino acid sequence set forth in SEQ ID NO:24). In some embodiments, the anti-CD73 antibodies comprise a heavy chain comprising a VH comprising the VH CDR1, VH CDR2, and VH CDR3 of HzCL25 (see Table 1, e.g., according to the IMGT definition, i.e., the amino acid sequences set forth in SEQ ID NOs: 1-3, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of HzCL25 (i.e., the amino acid sequence set forth in SEQ ID NO:22), wherein the heavy chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of HzCL25 (i.e., the amino acid sequence set forth in SEQ ID NO:24). In certain embodiments, the anti-CD73 antibodies comprise a heavy chain comprising the amino acid sequence set forth in SEQ ID NO:24. In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of HzCL25 (i.e., the amino acid sequence set forth in SEQ ID NO:23). In certain embodiments, the anti-CD73 antibodies comprise a VL comprising the VL CDR1, VL CDR2, and VL CDR3 of HzCL25 (see Table 1, e.g., according to the IMGT definition, i.e., the amino acid sequences set forth in SEQ ID NOs: 4-6, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of HzCL25 (i.e., the amino acid sequence set forth in SEQ ID NO:23). In certain embodiments, the anti-CD73 antibodies comprise a VL comprising the amino acid sequence set forth in SEQ ID NO:23. In some embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of HzCL25 (i.e., the amino acid sequence set forth in SEQ ID NO:25). In some embodiments, the anti-CD73 antibodies comprise a light chain comprising a VL comprising the VL CDR1, VL CDR2, and VL CDR3 of HzCL25 (see Table 1, e.g., according to the IMGT definition, i.e., the amino acid sequences set forth in SEQ ID NOs: 4-6, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of HzCL25 (i.e., the amino acid sequence set forth in SEQ ID NO:23), wherein the light chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of HzCL25 (i.e., the amino acid sequence set forth in SEQ ID NO:25). In certain embodiments, the anti-CD73 antibodies comprise a light chain comprising the amino acid sequence set forth in SEQ ID NO:25. In certain embodiments, the anti-CD73 antibodies comprise: (i) an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of HzCL25 (i.e., the amino acid sequence set forth in SEQ ID NO:22); and (ii) an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of HzCL25 (i.e., the amino acid sequence set forth in SEQ ID NO:23). In certain embodiments, the anti-CD73 antibodies comprise: (i) a VH comprising the VH CDR1, VH CDR2, and VH CDR3 of HzCL25 (see Table 1, e.g., according to the IMGT definition, i.e., the amino acid sequences set forth in SEQ ID NOs: 1-3, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of HzCL25 (i.e., the amino acid sequence set forth in SEQ ID NO:22), and (ii) a VL comprising the VL CDR1, VL CDR2, and VL CDR3 of HzCL25 (see Table 1, e.g., according to the IMGT definition, i.e., the amino acid sequences set forth in SEQ ID NOs: 4-6, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of HzCL25 (i.e., the amino acid sequence set forth in SEQ ID NO:23). In certain embodiments, the anti-CD73 antibodies comprise: a VH comprising the amino acid sequence set forth in SEQ ID NO:22, and (ii) a VL comprising the amino acid sequence set forth in SEQ ID NO:23. In some embodiments, the anti-CD73 antibodies comprise: (i) an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of HzCL25 (i.e., the amino acid sequence set forth in SEQ ID NO:24); and (ii) an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of HzCL25 (i.e., the amino acid sequence set forth in SEQ ID NO:25). In some embodiments, the anti-CD73 antibodies comprise: (i) a heavy chain comprising a VH comprising the VH CDR1, VH CDR2, and VH CDR3 of HzCL25 (see Table 1, e.g., according to the IMGT definition, i.e., the amino acid sequences set forth in SEQ ID NOs: 1-3, respectively), wherein the heavy chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of HzCL25 (i.e., the amino acid sequence set forth in SEQ ID NO:24), and (ii) a light chain comprising a VL comprising the VL CDR1, VL CDR2, and VL CDR3 of HzCL25 (see Table 1, e.g., according to the IMGT definition, i.e., the amino acid sequences set forth in SEQ ID NOs: 4-6, respectively), wherein the light chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of HzCL25 (i.e., the amino acid sequence set forth in SEQ ID NO:25). In some embodiments, the anti-CD73 antibodies comprise: (i) a heavy chain comprising the amino acid sequence set forth in SEQ ID NO:24, and (ii) a light chain comprising the amino acid sequence set forth in SEQ ID NO:25.

The CD73-binding epitope of HzCL25 is within the amino acid sequence TKVQQIRRAEPNVL (SEQ ID NO:76) (i.e., amino acids 40-53 of the amino acid sequence set forth in SEQ ID NO:70). This disclosure features antibodies that bind to CD73 within the sequence TKVQQIRRAEPNVL (SEQ ID NO:76). This disclosure features antibodies that bind to the same epitope as HzCL25. This disclosure also features antibodies that competitively inhibit binding of HzCL25 to human CD73.

In some embodiments, the VH of HzCL25 is linked to a heavy chain constant region comprising a CH1 domain and a hinge region. In some embodiments, the VH of HzCL25 is linked to a heavy chain constant region comprising a CH3 domain. In some embodiments, the CH3 domain lacks the C-terminal lysine (K) amino acid residue. In some embodiments, the CH3 domain contains the C-terminal lysine (K) amino acid residue. In certain embodiments, the VH of HzCL25 is linked to a heavy chain constant region comprising a CH1 domain, hinge region, CH2 domain, and CH3 domain from human IgG1. In some embodiments, the CH3 domain from human IgG1 lacks the C-terminal lysine (K) amino acid residue. In some embodiments, the CH3 domain from human IgG1 contains the C-terminal lysine (K) amino acid residue. In certain embodiments such an antibody contains one or more additional mutations in the heavy chain constant region that increase the stability of the antibody. In certain embodiments, the heavy chain constant region includes substitutions that modify the properties of the antibody (e.g., decrease Fc receptor binding, increase or decrease antibody glycosylation, decrease binding to C1q). In certain embodiments, the heavy chain constant region includes an alanine at position Asparagine-297 (N297, according to EU numbering) of the heavy chain constant region to reduce effector function.

In certain embodiments, the anti-CD73 antibody is an IgG antibody. In one embodiment, the antibody is an IgG1 antibody. In one embodiment, the antibody is an IgG4 antibody. In another embodiment, the antibody is an IgG2 antibody. In certain embodiments, the anti-CD73 antibody comprises a heavy chain constant region lacking one or more lysine (K) amino acid residues relative to a wild type heavy chain constant region. For example, in certain embodiments, the antibody comprises heavy chain constant region lacking the C-terminal lysine (K) amino acid residue of the CH3 domain of the heavy chain constant region. In certain embodiments, the anti-CD73 antibody comprises a heavy chain constant region having the amino acid sequence set forth in SEQ ID NO:73. In certain embodiments, the anti-CD73 antibody comprises a heavy chain constant region having the amino acid sequence set forth in SEQ ID NO:75. In certain embodiments, the anti-CD73 antibody comprises a light chain constant region having the amino acid sequence set forth in SEQ ID NO:74. In certain embodiments, the anti-CD73 antibody comprises a heavy chain constant region having the amino acid sequence set forth in SEQ ID NO:73 and a light chain constant region having the amino acid sequence set forth in SEQ ID NO:74. In certain embodiments, the anti-CD73 antibody comprises a heavy chain constant region having the amino acid sequence set forth in SEQ ID NO:75 and a light chain constant region having the amino acid sequence set forth in SEQ ID NO:74.

### Antibody 3-F03

Antibody 3-F03 is a human IgG1/kappa monoclonal antibody with alanine at position Asparagine-297 (N297, according to EU numbering) of the heavy chain constant region to reduce effector function. 3-F03 specifically binds human, cynomolgus, and murine CD73 with high affinity (K_{D}≤2 nM) and has low effector functionality.

3-F03 was engineered from sequences obtained by multiple selection rounds of single donor library. scFv cassettes from this pool were then recombined into a yeast display vector library, which was subjected to FACs selection with murine CD73 (SEQ ID NO:71). The amino acid sequences of the yeast 3-F03 scFv cassette are set forth in SEQ ID NOs:77 and 65, respectively:

(SEQ ID NO: 77)
EVQLVESGGGLVQPGGSLRLSCAASGFTFSSYDMHWVRQAPGKGLEWVAV
MSYDGSNKYYADSVKGRFTISRDNSKNTLYLQMNSLRAEDTAVYYCATEI
AAKGDYWGQGTLVTVSS;
and
(SEQ ID NO: 65)
AIQMTQSPSSLSASVGDRVTITCRASQGISNYLAWYQQKPGKAPKLLIYA
ASTLQSGVPSRFSGSGSGTDFTLTISSLQPEDFATYYCQQSYSTPHFGQG
TRLEIK

To construct the 3-F03 antibody, the yeast 3-F03 VH and VL were modified as follows and cloned into a human IgG1/kappa scaffold. For the VH, the N-terminal glutamate (E) of yeast 3-F03 VH (SEQ ID NO:77) was removed and the threonine (T) at Kabat position H77 of SEQ ID NO:77 (i.e., position 78 of SEQ ID NO:77) was substituted with an alanine (A). For the VL, the N-terminal alanine (A) of SEQ ID NO:65 was removed. The resulting full-length human 3-F03 antibody contains the VH and VL set forth in the amino acid sequences of SEQ ID NOs:60 and 61, respectively. The resulting full-length human 3-F03 antibody is referred to herein as “3-F03”. Table 3, below, shows the amino acid sequences of the 3-F03 CDRs according to IMGT, Chothia, AbM, Kabat, and Contact numbering. Table 3, below, also shows the amino acid sequences of the 3-F03 mature VH, VL, heavy chain, and light chain.

TABLE 3
Amino acid sequences of 3-F03 CDRs, VH, and VL

| | IMGT | Chothia | AbM | Kabat | Contact |
|---|---|---|---|---|---|
| VH | GFTFSSYD | GFTFSSY (SEQ | GFTFSSYDMH | SYDMH (SEQ | SSYDMH (SEQ |
| CDR1 | (SEQ ID NO: 34) | ID NO: 41) | (SEQ ID NO: 46) | ID NO: 49) | ID NO: 53) |
| VH | MSYDGSNK | SYDGSN (SEQ | VMSYDGSNKY | VMSYDGSNKY | WVAVMSYDGS |
| CDR2 | (SEQ ID | ID NO: 42) | (SEQ ID NO: 47) | YADSVKG (SEQ | NKY (SEQ ID |
| | NO: 35) | | | ID NO: 50) | NO: 54) |
| VH | ATEIAAKGDY | EIAAKGDY | EIAAKGDY | EIAAKGDY | ATEIAAKGD |
| CDR3 | (SEQ ID | (SEQ ID NO: 52) | (SEQ ID NO: 52) | (SEQ ID NO: 52) | (SEQ ID NO: 56) |
| | NO: 36) | | | | |
| VL | QGISNY (SEQ | RASQGISNYLA | RASQGISNYLA | RASQGISNYLA | SNYLAWY |
| CDR1 | ID NO: 37) | (SEQ ID NO: 44) | (SEQ ID NO: 44) | (SEQ ID NO: 44) | (SEQ ID NO: 57) |
| VL | AAS (SEQ ID | AASTL QS (SEQ | AASTL QS (SEQ | AASTLOS (SEQ | LLIYAASTLQ |
| CDR2 | NO: 38) | ID NO: 45) | ID NO: 45) | ID NO: 45) | (SEQ ID NO: 58) |
| VL | QQSYSTPH | QQSYSTPH | QQSYSTPH | QQSYSTPH | QQSYSTP (SEQ |
| CDR3 | (SEQ ID | (SEQ ID NO: 39) | (SEQ ID NO: 39) | (SEQ ID NO: 39) | ID NO: 59) |
| | NO: 39) | | | | |

| | |
|---|---|
| VH | VQLVESGGGLVQPGGSLRLSCAASGFTFSSYDMHWVRQAPGKGLEWVAVMSYDGSNKYYA |
| | DSVKGRFTISRDNSKNALYLQMNSLRAEDTAVYYCATEIAAKGDYWGQGTLVTVSS (SEQ |
| | ID NO: 60) |
| VL | IQMTQSPSSLSASVGDRVTITCRASQGISNYLAWYQQKPGKAPKLLIYAASTLQSGVPSRFSGS |
| | GSGTDFTLTISSLQPEDFATYYCQQSYSTPHFGQGTRLEIK (SEQ ID NO: 61) |
| HC | VQLVESGGGLVQPGGSLRLSCAASGFTFSSYDMHWVRQAPGKGLEWVAVMSYDGSNKYYA |
| | DSVKGRFTISRDNSKNALYLQMNSLRAEDTAVYYCATEIAAKGDYWGQGTLVTVSSASTKG |
| | PSVFPLAPSSKSTSGGTAALGCLVKDYFPEPVTVSWNSGALTSGVHTFPAVLQSSGLYSLSSV |
| | VTVPSSSLGTQTYICNVNHKPSNTKVDKRVEPKSCDKTHTCPPCPAPELLGGPSVFLFPPKPKD |
| | TLMISRTPEVTCVVVDVSHEDPEVKFNWYVDGVEVHNAKTKPREEQYASTYRVVSVLTVLH |
| | QDWLNGKEYKCKVSNKALPAPIEKTISKAKGQPREPQVYTLPPSREEMTKNQVSLTCLVKGF |
| | YPSDIAVEWESNGQPENNYKTTPPVLDSDGSFFLYSKLTVDKSRWQQGNVFSCSVMHEALHN |
| | HYTQKSLSLSPGK (SEQ ID NO: 66) |
| LC | IQMTQSPSSLSASVGDRVTITCRASQGISNYLAWYQQKPGKAPKLLIYAASTLQSGVPSRFSGS |
| | GSGTDFTLTISSLQPEDFATYYCQQSYSTPHFGQGTRLEIKRTVAAPSVFIFPPSDEQLKSGTAS |
| | VVCLLNNFYPREAKVQWKVDNALQSGNSQESVTEQDSKDSTYSLSSTLTLSKADYEKHKVY |
| | ACEVTHQGLSSPVTKSFNRGEC (SEQ ID NO: 31) |

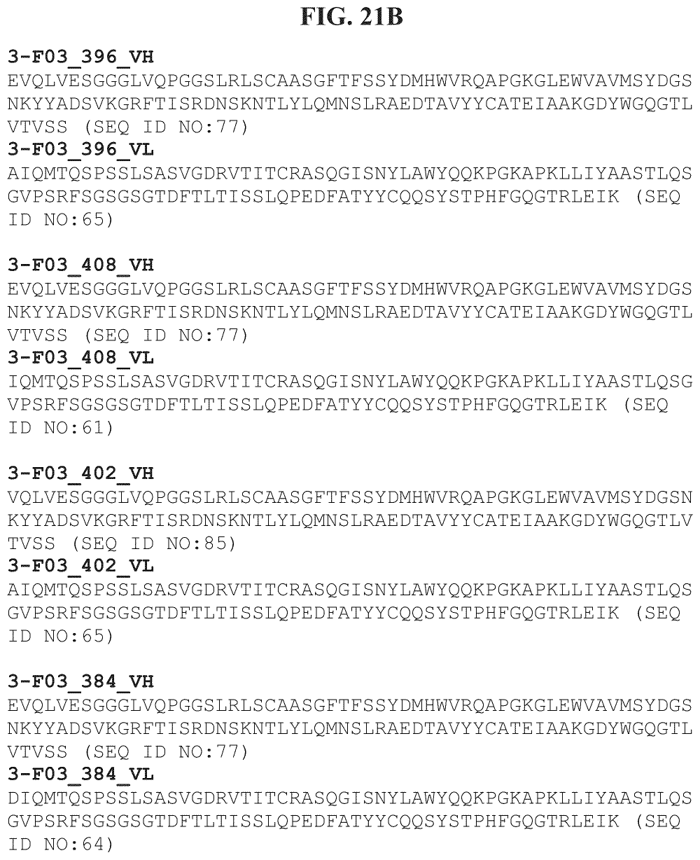

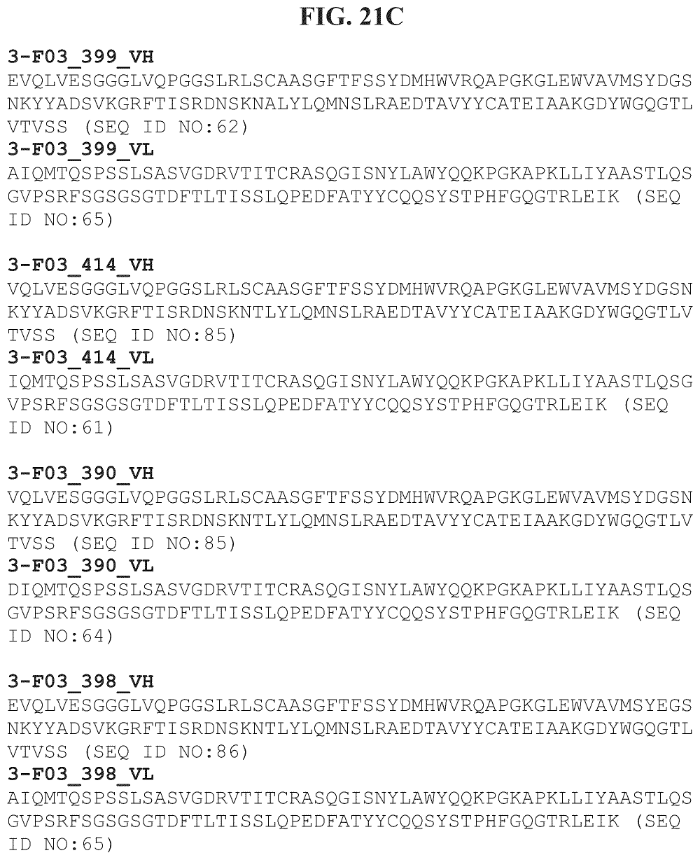

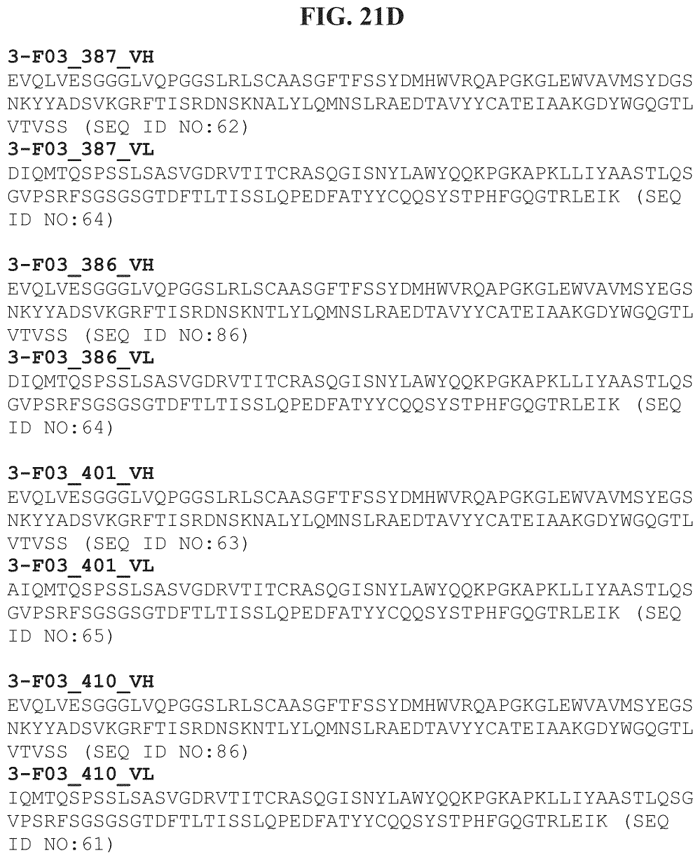

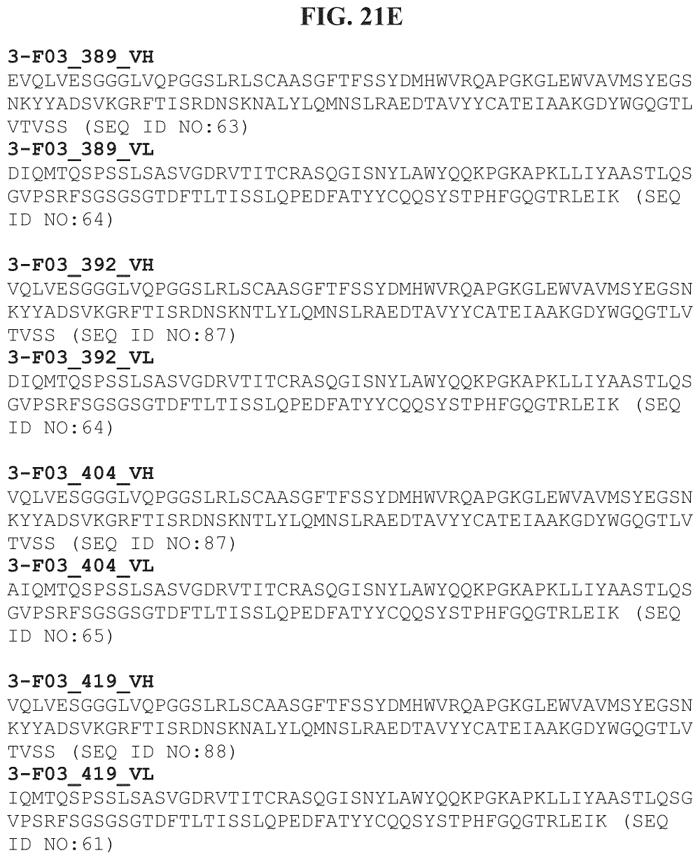

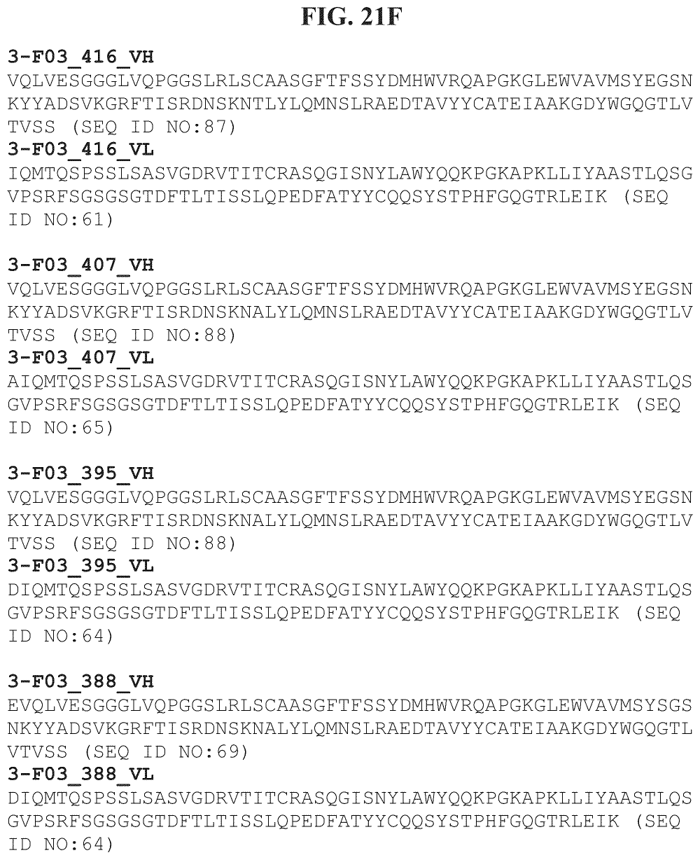

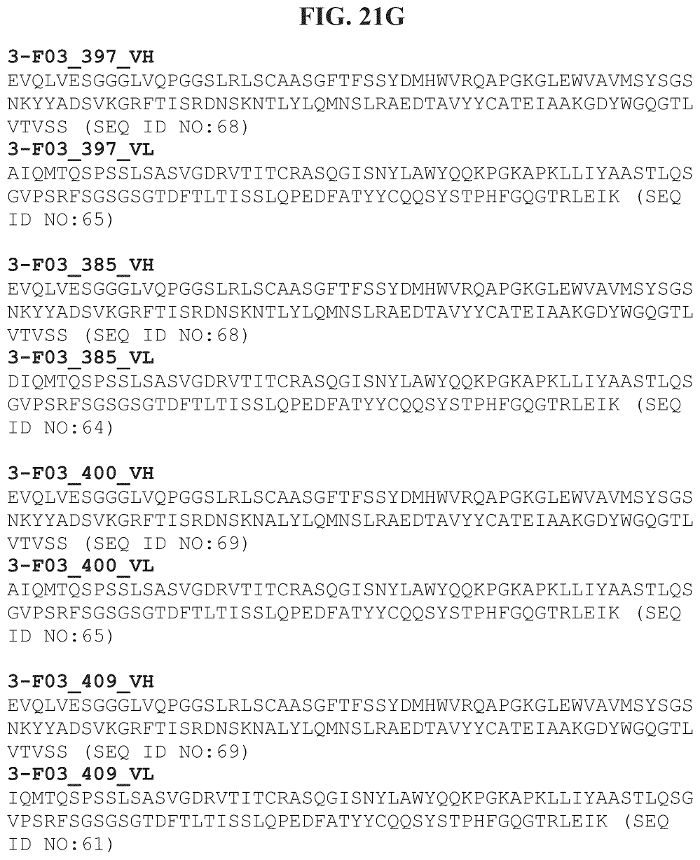

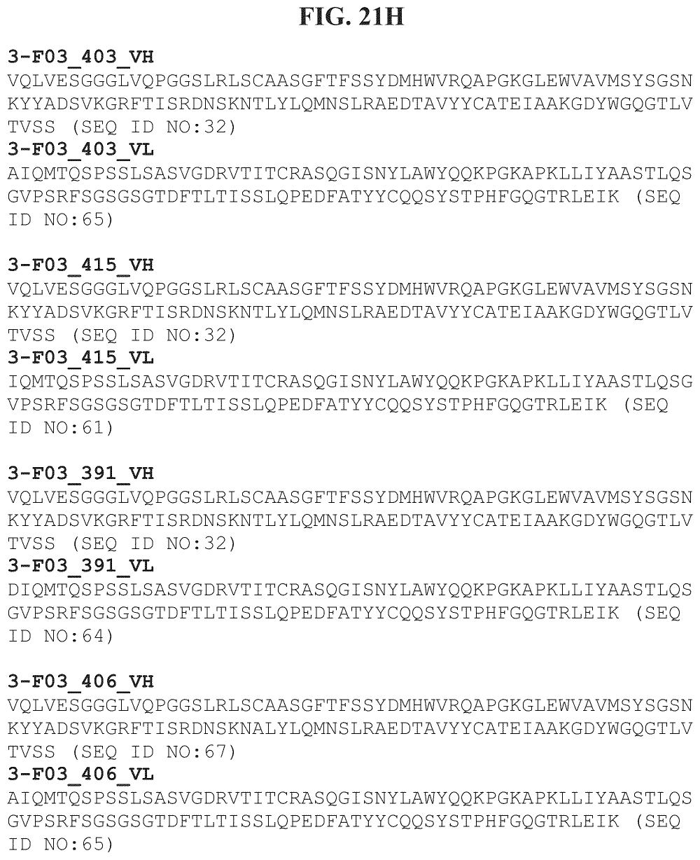

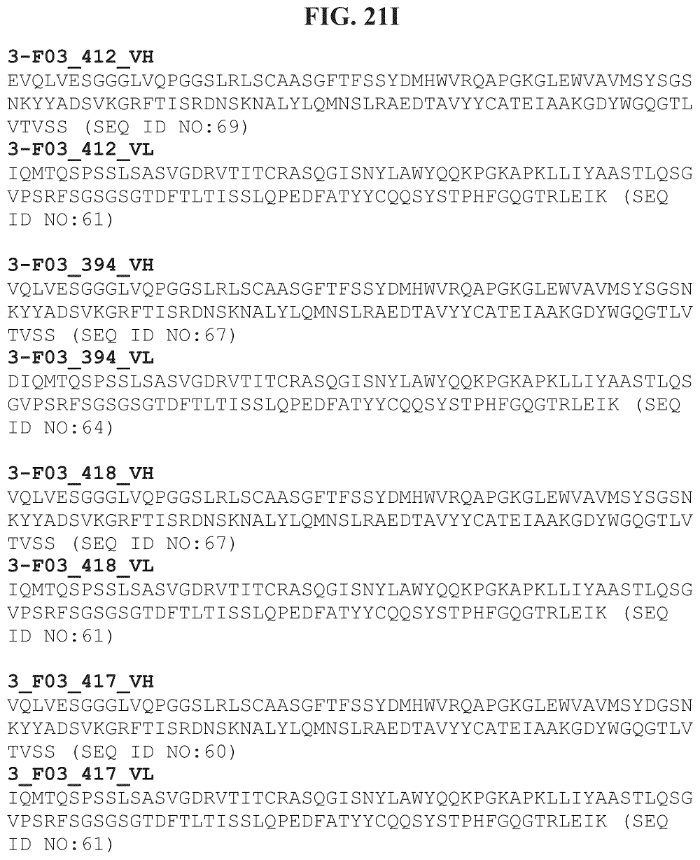

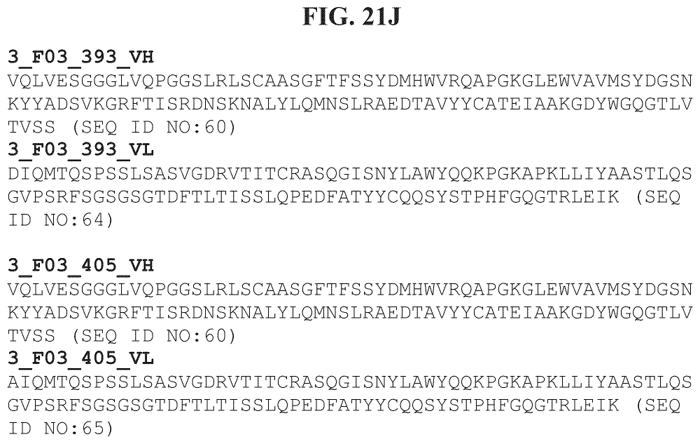

Variants of 3-F03 are also described herein. 3-F03_411 is identical to 3-F03, except that the 3-F03_411 heavy chain (i) contains an N-terminal glutamate (E) that is lacking in 3-F03 and (ii) does not include the C-terminal lysine present in 3-F03. Table 4, below, shows the amino acid sequences of the 3-F03_411 mature VH, VL, heavy chain and light chain. 3-F03_413 is identical to 3-F03_411, except that it contains a glutamate (E) at VH Kabat position H53 (position 54 of SEQ ID NO:60) instead of an aspartic acid (D). Table 5, below, shows the amino acid sequences of the 3-F03_413 CDRs according to IMGT, Chothia, AbM, Kabat, and Contact numbering. Table 5, below, also shows the amino acid sequences of the 3-F03_413 mature VH, VL, heavy chain, and light chain. Additional variants are described in the Examples below (see FIG. 21A-FIG. 21J).

TABLE 4
Amino acid sequences of 3-F03_411 HC and LC

| | SEQUENCE |
|---|---|
| VH | EVQLVESGGGLVQPGGSLRLSCAASGFTFSSYDMHWVRQAPGKGLEWVAVMSYDGSNKYY |
| | ADSVKGRFTISRDNSKNALYLQMNSLRAEDTAVYYCATEIAAKGDYWGQGTLVTVSS (SEQ |
| | ID NO: 62) |
| VL | IQMTQSPSSLSASVGDRVTITCRASQGISNYLAWYQQKPGKAPKLLIYAASTLQSGVPSRFSGS |
| | GSGTDFTLTISSLQPEDFATYYCQQSYSTPHFGQGTRLEIK (SEQ ID NO: 61) |
| Heavy | EVQLVESGGGLVQPGGSLRLSCAASGFTFSSYDMHWVRQAPGKGLEWVAVMSYDGSNKYY |
| Chain | ADSVKGRFTISRDNSKNALYLQMNSLRAEDTAVYYCATEIAAKGDYWGQGTLVTVSSASTK |
| | GPSVFPLAPSSKSTSGGTAALGCLVKDYFPEPVTVSWNSGALTSGVHTFPAVLQSSGLYSLSS |
| | VVTVPSSSLGTQTYICNVNHKPSNTKVDKRVEPKSCDKTHTCPPCPAPELLGGPSVFLFPPKPK |
| | DTLMISRTPEVTCVVVDVSHEDPEVKFNWYVDGVEVHNAKTKPREEQYASTYRVVSVLTVL |
| | HQDWLNGKEYKCKVSNKALPAPIEKTISKAKGQPREPQVYTLPPSREEMTKNQVSLTCLVKG |
| | FYPSDIAVEWESNGQPENNYKTTPPVLDSDGSFFLYSKLTVDKSRWQQGNVFSCSVMHEALH |
| | NHYTQKSLSLSPG (SEQ ID NO: 30) |
| Light | IQMTQSPSSLSASVGDRVTITCRASQGISNYLAWYQQKPGKAPKLLIYAASTLQSGVPSRFSGS |
| Chain | GSGTDFTLTISSLQPEDFATYYCQQSYSTPHFGQGTRLEIKRTVAAPSVFIFPPSDEQLKSGTAS |
| | VVCLLNNFYPREAKVQWKVDNALQSGNSQESVTEQDSKDSTYSLSSTLTLSKADYEKHKVY |
| | ACEVTHQGLSSPVTKSFNRGEC (SEQ ID NO: 31) |

TABLE 5
Amino acid sequences of 3-F03_413 CDRs, VH, VL, HC, LC

| | IMGT | Chothia | AbM | Kabat | Contact |
|---|---|---|---|---|---|
| VH | GFTFSSYD | GFTFSSY (SEQ | GFTFSSYDMH | SYDMH (SEQ | SSYDMH (SEQ |
| CDR1 | (SEQ ID NO: 34) | ID NO: 41) | (SEQ ID NO: 46) | ID NO: 49) | ID NO: 53) |
| VH | MSYEGSNK | SYEGSN (SEQ | VMSYEGSNKY | VMSYEGSNKY | WVAVMSYEGS |
| CDR2 | (SEQ ID | ID NO: 43) | (SEQ ID NO: 48) | YADSVKG (SEQ | NKY (SEQ ID |
| | NO: 40) | | | ID NO: 51) | NO: 55) |
| VH | ATEIAAKGDY | EIAAKGDY | EIAAKGDY | EIAAKGDY | ATEIAAKGD |
| CDR3 | (SEQ ID | (SEQ ID NO: 52) | (SEQ ID NO: 52) | (SEQ ID NO: 52) | (SEQ ID NO: 56) |
| | NO: 36) | | | | |
| VL | QGISNY (SEQ | RASQGISNYLA | RASQGISNYLA | RASQGISNYLA | SNYLAWY |
| CDR1 | ID NO: 37) | (SEQ ID NO: 44) | (SEQ ID NO: 44) | (SEQ ID NO: 44) | (SEQ ID NO: 57) |
| VL | AAS (SEQ ID | AASTLQS (SEQ | AASTLQS (SEQ | AASTLQS (SEQ | LLIYAASTLQ |
| CDR2 | NO: 38) | ID NO: 45) | ID NO: 45) | ID NO: 45) | (SEQ ID NO: 58) |
| VL | QQSYSTPH | QQSYSTPH | QQSYSTPH | QQSYSTPH | QQSYSTP (SEQ |
| CDR3 | (SEQ ID | (SEQ ID NO: 39) | (SEQ ID NO: 39) | (SEQ ID NO: 39) | ID NO: 59) |
| | NO: 39) | | | | |

| | |
|---|---|
| VH | EVQLVESGGGLVQPGGSLRLSCAASGFTFSSYDMHWVRQAPGKGLEWVAVMSYEGSNKYY |
| | ADSVKGRFTISRDNSKNALYLQMNSLRAEDTAVYYCATEIAAKGDYWGQGTLVTVSS (SEQ |
| | ID NO: 63) |
| VL | IQMTQSPSSLSASVGDRVTITCRASQGISNYLAWYQQKPGKAPKLLIYAASTLQSGVPSRFSGS |
| | GSGTDFTLTISSLQPEDFATYYCQQSYSTPHFGQGTRLEIK (SEQ ID NO: 61) |
| HC | EVQLVESGGGLVQPGGSLRLSCAASGFTFSSYDMHWVRQAPGKGLEWVAVMSYEGSNKYY |
| | ADSVKGRFTISRDNSKNALYLQMNSLRAEDTAVYYCATEIAAKGDYWGQGTLVTVSSASTK |
| | GPSVFPLAPSSKSTSGGTAALGCLVKDYFPEPVTVSWNSGALTSGVHTFPAVLQSSGLYSLSS |
| | VVTVPSSSLGTQTYICNVNHKPSNTKVDKRVEPKSCDKTHTCPPCPAPELLGGPSVFLFPPKPK |
| | DTLMISRTPEVTCVVVDVSHEDPEVKFNWYVDGVEVHNAKTKPREEQYASTYRVVSVLTVL |
| | HQDWLNGKEYKCKVSNKALPAPIEKTISKAKGQPREPQVYTLPPSREEMTKNQVSLTCLVKG |
| | FYPSDIAVEWESNGQPENNYKTTPPVLDSDGSFFLYSKLTVDKSRWQQGNVFSCSVMHEALH |
| | NHYTQKSLSLSPG (SEQ ID NO: 33) |
| LC | IQMTQSPSSLSASVGDRVTITCRASQGISNYLAWYQQKPGKAPKLLIYAASTLQSGVPSRFSGS |
| | GSGTDFTLTISSLQPEDFATYYCQQSYSTPHFGQGTRLEIKRTVAAPSVFIFPPSDEQLKSGTAS |
| | VVCLLNNFYPREAKVQWKVDNALQSGNSQESVTEQDSKDSTYSLSSTLTLSKADYEKHKVY |
| | ACEVTHQGLSSPVTKSFNRGEC (SEQ ID NO: 31) |

The anti-CD73 antibodies can encompass the VH CDR1, VH CDR2, and VH CDR3 and the VL CDR1, VL CDR2, and VL CDR3 of 3-F03 or 3-F03_413. In some instances, the anti-CD73 antibody comprises a VH comprising VH CDR1, VH CDR2, and VH CDR3 of 3-F03 (see Table 3). In some instances, the anti-CD73 antibody comprises a VL comprising VL CDR1, VL CDR2, and VL CDR3 of 3-F03 (see Table 3). In some instances, the anti-CD73 antibody comprises a VH comprising VH CDR1, VH CDR2, and VH CDR3 of 3-F03 (see Table 3) and a VL comprising VL CDR1, VL CDR2, and VL CDR3 of 3-F03 (see Table 3). In some instances, the anti-CD73 antibody comprises a VH comprising VH CDR1, VH CDR2, and VH CDR3 of 3-F03_413 (see Table 5). In some instances, the anti-CD73 antibody comprises a VL comprising VL CDR1, VL CDR2, and VL CDR3 of 3-F03_413 (see Table 5). In some instances, the anti-CD73 antibody comprises a VH comprising VH CDR1, VH CDR2, and VH CDR3 of 3-F03_413 (see Table 5) and a VL comprising VL CDR1, VL CDR2, and VL CDR3 of 3-F03_413 (see Table 5). In some instances, the anti-CD73 antibodies can have, e.g., 1, 2, or 3 substitutions within one or more (i.e., 1, 2, 3, 4, 5, or 6) of the six CDRs of 3-F03 or 3-F03_413. In some instances, these antibodies (i) inhibit cellular CD73 (e.g., at least 10%; at least 20%, at least 30%, at least 40%, at least 50%, at least 60%, at least 70%, at least 80%, at least 90%, at least 95%, at least 97%, at least 99%, or 100% reduction in cellular CD73 activity as compared to an isotype control as determined by, e.g., the assay described in Example 15); and/or (ii) inhibit soluble CD73 (e.g., at least 10%; at least 20%, at least 30%, at least 40%, at least 50%, at least 60%, at least 70%, at least 80%, at least 90%, at least 95%, at least 97%, at least 99%, or 100% reduction in soluble CD73 activity as compared to an isotype control as determined by, e.g., the assay described in Example 16); and/or (iii) bind human, cynomolgus monkey, or murine CD73 in the open conformation with high affinity (e.g., K_{D}≤2 nM) (e.g., as determined by the binding assay described in Example 18); and/or (iv) do not bind human, cynomolgus monkey, or murine CD73 in the closed conformation; and/or (v) bind to an epitope within amino acids 386-399 of SEQ ID NO:70 (i.e., within AAVLPFGGTFDLVQ (SEQ ID NO:78) amino acids 470-489 of SEQ ID NO:70 (i.e., within ILPNFLANGGDGFQMIKDEL (SEQ ID NO:79)) (e.g., as determined by the binding assay described in Example 6); and/or (vi) reduce AMP-mediated suppression of T cell proliferation (e.g., at least 10%; at least 20%, at least 30%, at least 40%, at least 50%, at least 60%, at least 70%, at least 80%, at least 90%, at least 95%, at least 97%, at least 99%, or 100% reduction in T cell proliferation as compared to an isotype control as determined by, e.g., the assay described in Example 17); and/or (vii) decreases levels of cell surface CD73 (e.g., on cancer cells, e.g., on melanoma cancer cells, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control as determined by, e.g., the assay described in Example 20); and/or (viii) reduce tumor growth (e.g., melanoma tumors, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control as determined by, e.g., the assay described in Example 21).

The anti-CD73 antibodies can comprise the VH CDR1, VH CDR2, and VH CDR3 of 3-F03 or 3-F03_413 according to the IMGT definition, or an alternate CDR definition such as, but not limited to, the Kabat definition, the Chothia definition, the AbM CDR definition, or the contact definition. These anti-CD73 antibodies may include zero, one, two, or three substitutions in VH CDR1 and/or VH CDR2 and/or VH CDR3 of 3-F03 or 3-F03_413. In some embodiments, the anti-CD73 antibodies further comprise the VL CDR1, VL CDR2, and VL CDR3 of 3-F03 or 3-F03_413 according to the IMGT definition, or an alternate CDR definition such as, but not limited to, the Kabat definition, the Chothia definition, the AbM CDR definition, or the contact definition. These anti-CD73 antibodies may include zero, one, two, or three substitutions in VL CDR1 and/or VL CDR2 and/or VL CDR3 of 3-F03 or 3-F03_413. In some instances, the anti-CD73 antibody comprises a VH comprising the VH CDR1, VH CDR2, and VH CDR3 set forth in SEQ ID NOs: 34, 35, and 36, respectively, and a VL comprising the VL CDR1, VL CDR2, and VL CDR3 set forth in SEQ ID NOs: 37, 38, and 39, respectively. In some instances, the anti-CD73 antibody comprises a VH comprising the VH CDR1, VH CDR2, and VH CDR3 set forth in SEQ ID NOs: 41, 42, and 52, respectively, and a VL comprising the VL CDR1, VL CDR2, and VL CDR3 set forth in SEQ ID NOs: 44, 45, and 39, respectively. In some instances, the anti-CD73 antibody comprises a VH comprising the VH CDR1, VH CDR2, and VH CDR3 set forth in SEQ ID NOs: 46, 47, and 52, respectively, and a VL comprising the VL CDR1, VL CDR2, and VL CDR3 set forth in SEQ ID NOs: 44, 45, and 39, respectively. In some instances, the anti-CD73 antibody comprises a VH comprising the VH CDR1, VH CDR2, and VH CDR3 set forth in SEQ ID NOs: 49, 50, and 52, respectively, and a VL comprising the VL CDR1, VL CDR2, and VL CDR3 set forth in SEQ ID NOs: 44, 45, and 39, respectively. In some instances, the anti-CD73 antibody comprises a VH comprising the VH CDR1, VH CDR2, and VH CDR3 set forth in SEQ ID NOs: 53, 54, and 56, respectively, and a VL comprising the VL CDR1, VL CDR2, and VL CDR3 set forth in SEQ ID NOs: 57, 58, and 59, respectively. In some instances, the anti-CD73 antibody comprises a VH comprising the VH CDR1, VH CDR2, and VH CDR3 set forth in SEQ ID NOs: 34, 40, and 36, respectively, and a VL comprising the VL CDR1, VL CDR2, and VL CDR3 set forth in SEQ ID NOs: 37, 38, and 39, respectively. In some instances, the anti-CD73 antibody comprises a VH comprising the VH CDR1, VH CDR2, and VH CDR3 set forth in SEQ ID NOs: 41, 43, and 52, respectively, and a VL comprising the VL CDR1, VL CDR2, and VL CDR3 set forth in SEQ ID NOs: 44, 45, and 39, respectively. In some instances, the anti-CD73 antibody comprises a VH comprising the VH CDR1, VH CDR2, and VH CDR3 set forth in SEQ ID NOs: 46, 48, and 52, respectively, and a VL comprising the VL CDR1, VL CDR2, and VL CDR3 set forth in SEQ ID NOs: 44, 45, and 39, respectively. In some instances, the anti-CD73 antibody comprises a VH comprising the VH CDR1, VH CDR2, and VH CDR3 set forth in SEQ ID NOs: 49, 51, and 52, respectively, and a VL comprising the VL CDR1, VL CDR2, and VL CDR3 set forth in SEQ ID NOs: 44, 45, and 39, respectively. In some instances, the anti-CD73 antibody comprises a VH comprising the VH CDR1, VH CDR2, and VH CDR3 set forth in SEQ ID NOs: 53, 55, and 56, respectively, and a VL comprising the VL CDR1, VL CDR2, and VL CDR3 set forth in SEQ ID NOs: 57, 58, and 59, respectively. In some instances, these antibodies (i) inhibit cellular CD73 (e.g., at least 10%; at least 20%, at least 30%, at least 40%, at least 50%, at least 60%, at least 70%, at least 80%, at least 90%, at least 95%, at least 97%, at least 99%, or 100% reduction in cellular CD73 activity as compared to an isotype control as determined by, e.g., the assay described in Example 15); and/or (ii) inhibit soluble CD73 (e.g., at least 10%; at least 20%, at least 30%, at least 40%, at least 50%, at least 60%, at least 70%, at least 80%, at least 90%, at least 95%, at least 97%, at least 99%, or 100% reduction in soluble CD73 activity as compared to an isotype control as determined by, e.g., the assay described in Example 16); and/or (iii) bind human, cynomolgus monkey, or murine CD73 in the open conformation with high affinity (e.g., K_{D}≤2 nM) (e.g., as determined by the binding assay described in Example 18); and/or (iv) do not bind human, cynomolgus monkey, or murine CD73 in the closed conformation; and/or (v) bind to an epitope within amino acids 386-399 of SEQ ID NO:70 (i.e., within AAVLPFGGTFDLVQ (SEQ ID NO:78) amino acids 470-489 of SEQ ID NO:70 (i.e., within ILPNFLANGGDGFQMIKDEL (SEQ ID NO:79)) (e.g., as determined by the binding assay described in Example 6); and/or (vi) reduce AMP-mediated suppression of T cell proliferation (e.g., at least 10%; at least 20%, at least 30%, at least 40%, at least 50%, at least 60%, at least 70%, at least 80%, at least 90%, at least 95%, at least 97%, at least 99%, or 100% reduction in T cell proliferation as compared to an isotype control as determined by, e.g., the assay described in Example 17); and/or (vii) decreases levels of cell surface CD73 (e.g., on cancer cells, e.g., on melanoma cancer cells, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control as determined by, e.g., the assay described in Example 20); and/or (viii) reduce tumor growth (e.g., melanoma tumors, e.g., by at least 10%; at least 20%, at least 30%, at least 40%, at least 50%, at least 60%, at least 70%, at least 80%, at least 90%, at least 95%, at least 97%, at least 99%, or 100% as compared to an isotype control as determined by, e.g., the assay described in Example 21).

In certain embodiments, the anti-CD73 antibodies comprise an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 32, 60, 62, 63, 67-69, 77, and 85-88. In certain embodiments, the anti-CD73 antibodies comprise a VH comprising the VH CDR1, VH CDR2, and VH CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs: 34-36, respectively, or SEQ ID NOs:34, 40 and 36, respectively), wherein the VH comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 32, 60, 62, 63, 67-69, 77, and 85-88. In some embodiments, the anti-CD73 antibodies comprise an amino acid sequence having one or more (e.g., 1, 2, 3, 4, 5, 6, 7, 8, 9, 10) amino acid substitutions, additions, and/or deletions relative to the amino acid sequence set forth in SEQ ID NO: 30, 33, and 66. In some embodiments, the anti-CD73 antibodies comprise a heavy chain comprising the VH CDR1, VH CDR2, and VH CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs:34-36, respectively, or SEQ ID NOs:34, 40, and 36, respectively), wherein the heavy chain comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO: 30, 33, and 66. In certain embodiments, the anti-CD73 antibodies comprise an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 61, 64, and 65. In certain embodiments, the anti-CD73 antibodies comprise a VL comprising the VL CDR1, VL CDR2, and VL CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs: 37-39, respectively), wherein the VL comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 61, 64, and 65. In some embodiments, the anti-CD73 antibodies comprise an amino acid sequence having one or more (e.g., 1, 2, 3, 4, 5, 6, 7, 8, 9, 10) amino acid substitutions, additions, and/or deletions relative to the amino acid sequence set forth in SEQ ID NO: 31. In some embodiments, the anti-CD73 antibodies comprise a light chain comprising the VL CDR1, VL CDR2, and VL CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs:37-39, respectively), wherein the light chain comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO: 31. In certain embodiments, the anti-CD73 antibodies comprise: (i) an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 32, 60, 62, 63, 67-69, 77, and 85-88; and (ii) an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 61, 64, and 65. In certain embodiments, the anti-CD73 antibodies comprise: (i) a VH comprising the VH CDR1, VH CDR2, and VH CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs:34-36, respectively, or SEQ ID NOs: 34, 40, and 36, respectively), wherein the VH comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 32, 60, 62, 63, 67-69, 77, and 85-88; and (ii) a VL comprising the VL CDR1, VL CDR2, and VL CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs: 37-39, respectively), wherein the VL comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 61, 64, and 65. In some embodiments, the anti-CD73 antibodies comprise: (i) an amino acid sequence having one or more (e.g., 1, 2, 3, 4, 5, 6, 7, 8, 9, 10) amino acid substitutions, additions, and/or deletions relative to the amino acid sequence set forth in SEQ ID NOs: 30, 33, and 66; and (ii) an amino acid sequence having one or more (e.g., 1, 2, 3, 4, 5, 6, 7, 8, 9, 10) amino acid substitutions, additions, and/or deletions relative to the amino acid sequence set forth in SEQ ID NO: 31. In some embodiments, the anti-CD73 antibodies comprise: (i) a heavy chain comprising the VH CDR1, VH CDR2, and VH CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs: 34-36, respectively, or SEQ ID NOs:34, 40, and 36, respectively), wherein the heavy chain comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO: 30, 33, and 66; and (ii) a light chain comprising the VL CDR1, VL CDR2, and VL CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs:37-39, respectively), wherein the light chain comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO: 31.

In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH set forth in any one of SEQ ID NOs: 32, 60, 62, 63, 67-69, 77, and 85-88. In certain embodiments, the anti-CD73 antibodies comprise a VH comprising the VH CDR1, VH CDR2, and VH CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs:34-36, respectively, or SEQ ID NOs:34, 40, and 36, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH set forth in any one of SEQ ID NOs: 32, 60, 62, 63, 67-69, 77, and 85-88. In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL set forth in any one of SEQ ID NOs: 61, 64, and 65. In certain embodiments, the anti-CD73 antibodies comprise a VL comprising the VL CDR1, VL CDR2, and VL CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs:37-39, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL set forth in any one of SEQ ID NOs: 61, 64, and 65. In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH set forth in any one of SEQ ID NOs: 32, 60, 62, 63, 67-69, 77, and 85-88 and an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL set forth in SEQ ID NO: 61, 64, and 65. In certain embodiments, the anti-CD73 antibodies comprise: (i) a VH comprising the VH CDR1, VH CDR2, and VH CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs:34-36, respectively, or SEQ ID NOs:34, 40, and 36, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH set forth in any one of SEQ ID NOs: 32, 60, 62, 63, 67-69, 77, and 85-88, and (ii) a VL comprising the VL CDR1, VL CDR2, and VL CDR3 of 3-F03 or a variant thereof (e.g., 3-F03_411 or 3-F03_413) (see, e.g., Table 3 and Table 5, e.g., according to the IMGT definition, e.g., the amino acid sequences set forth in SEQ ID NOs:37-39, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL set forth in any one of SEQ ID NOs: 61, 64, and 65.

In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of 3-F03_411 or 3-F03_413 (i.e., the amino acid sequence set forth in SEQ ID NO:62 or 63, respectively). In certain embodiments, the anti-CD73 antibodies comprise a VH comprising the VH CDR1, VH CDR2, and VH CDR3 of 3-F03_411 (see Table 3, e.g., according to the IMGT definition, i.e., the amino acid sequences set forth in SEQ ID NOs: 34-36, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of 3-F03_411 (i.e., the amino acid sequence set forth in SEQ ID NO:62). In certain embodiments, the anti-CD73 antibodies comprise a VH comprising the VH CDR1, VH CDR2, and VH CDR3 of 3-F03_413 (see Table 5, e.g., according to the IMGT definition, i.e., the amino acid sequences set forth in SEQ ID NOs: 34, 40, and 36, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of 3-F03_411 (i.e., the amino acid sequence set forth in SEQ ID NO:63). In some embodiments, the anti-CD73 antibodies comprise a VH comprising the amino acid sequence set forth in SEQ ID NO:62. In some embodiments, the anti-CD73 antibodies comprise a VH comprising the amino acid sequence set forth in SEQ ID NO:63. In some embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of 3-F03_411 or 3-F03_F13 (i.e., the amino acid sequence set forth in SEQ ID NO:30 or 33, respectively). In some embodiments, the anti-CD73 antibodies comprise a heavy chain comprising a VH comprising the VH CDR1, VH CDR2, and VH CDR3 of 3-F03_411 (see Table 3, e.g., according to the IMGT definition, i.e., the amino acid sequences set forth in SEQ ID NOs: 34-36, respectively), wherein the heavy chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of 3-F03_411 (i.e., the amino acid sequence set forth in SEQ ID NO:30). In some embodiments, the anti-CD73 antibodies comprise a heavy chain comprising a VH comprising the VH CDR1, VH CDR2, and VH CDR3 of 3-F03_413 (see Table 5, e.g., according to the IMGT definition, i.e., the amino acid sequences set forth in SEQ ID NOs: 34, 40, and 36, respectively), wherein the heavy chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of 3-F03_413 (i.e., the amino acid sequence set forth in SEQ ID NO:33). In some embodiments, the anti-CD73 antibodies comprise a heavy chain comprising the amino acid sequence set forth in SEQ ID NO:30. In some embodiments, the anti-CD73 antibodies comprise a heavy chain comprising the amino acid sequence set forth in SEQ ID NO:33. In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of 3-F03_411 or 3-F03_413 (i.e., the amino acid sequence set forth in SEQ ID NO:61). In certain embodiments, the anti-CD73 antibodies comprise a VL comprising the VL CDR1, VL CDR2, and VL CDR3 of 3-F03_411 or 3-F03_413 (see Table 3, e.g., according to the IMGT definition, i.e., the amino acid sequences set forth in SEQ ID NOs: 37-39, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of 3-F03_411 or 3-F03_413 (i.e., the amino acid sequence set forth in SEQ ID NO:61). In some embodiments, the anti-CD73 antibodies comprise a VL comprising the amino acid sequence set forth in SEQ ID NO:61. In some embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of 3-F03_411 or 3-F03_413 (i.e., the amino acid sequence set forth in SEQ ID NO:31). In some embodiments, the anti-CD73 antibodies comprise a light chain comprising a VL comprising the VL CDR1, VL CDR2, and VL CDR3 of 3-F03_411 or 3-F03_413 (see Table 5, e.g., according to the IMGT definition, i.e., the amino acid sequences set forth in SEQ ID NOs: 37-39, respectively), wherein the light chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of 3-F03_411 or 3-F03_413 (i.e., the amino acid sequence set forth in SEQ ID NO:31). In some embodiments, the anti-CD73 antibodies comprise a light chain comprising the amino acid sequence set forth in SEQ ID NO:31. In certain embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of 3-F03_411 or 3-F03_413 (i.e., the amino acid sequence set forth in SEQ ID NO:62 or 63, respectively) and an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of 3-F03_411 or 3-F03_413 (i.e., the amino acid sequence set forth in SEQ ID NO:61). In certain embodiments, the anti-CD73 antibodies comprise: (i) a VH comprising the VH CDR1, VH CDR2, and VH CDR3 of 3-F03_411 (see Table 3, e.g., according to the IMGT definition, i.e., the amino acid sequences set forth in SEQ ID NOs: 34-36, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of 3-F03 (i.e., the amino acid sequence set forth in SEQ ID NO:62), and (ii) a VL comprising the VL CDR1, VL CDR2, and VL CDR3 of 3-F03_411 (see Table 3, e.g., according to the IMGT definition, i.e., the amino acid sequences set forth in SEQ ID NOs: 37-39, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of 3-F03 (i.e., the amino acid sequence set forth in SEQ ID NO:61). In certain embodiments, the anti-CD73 antibodies comprise: (i) a VH comprising the VH CDR1, VH CDR2, and VH CDR3 of 3-F03_413 (see Table 5, e.g., according to the IMGT definition, i.e., the amino acid sequences set forth in SEQ ID NOs: 34, 40, and 36, respectively), wherein the VH comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VH of 3-F03_413 (i.e., the amino acid sequence set forth in SEQ ID NO:63), and (ii) a VL comprising the VL CDR1, VL CDR2, and VL CDR3 of 3-F03_413 (see Table 5, e.g., according to the IMGT definition, i.e., the amino acid sequences set forth in SEQ ID NOs: 37-39, respectively), wherein the VL comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the VL of 3-F03_413 (i.e., the amino acid sequence set forth in SEQ ID NO:61). In some embodiments, the anti-CD73 antibody comprises: (i) a VH comprising the amino acid sequence set forth in SEQ ID NO:62; and (ii) a VL comprising the amino acid sequence set forth in SEQ ID NO:61. In some embodiments, the anti-CD73 antibody comprises: (i) a VH comprising the amino acid sequence set forth in SEQ ID NO:63; and (ii) a VL comprising the amino acid sequence set forth in SEQ ID NO:61. In some embodiments, the anti-CD73 antibodies comprise an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of 3-F03_411 or 3-F03_413 (i.e., the amino acid sequence set forth in SEQ ID NO:30 or 33) and an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of 3-F03_411 or 3-F03_413 (i.e., the amino acid sequence set forth in SEQ ID NO:31). In some embodiments, the anti-CD73 antibodies comprise: (i) a heavy chain comprising the a VH comprising the VH CDR1, VH CDR2, and VH CDR3 of 3-F03_411 (see Table 3, e.g., according to the IMGT definition, i.e., the amino acid sequences set forth in SEQ ID NOs: 34-36, respectively), wherein the heavy chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of 3-F03_411 (i.e., the amino acid sequence set forth in SEQ ID NO:30), and (ii) a light chain comprising a VL comprising the VL CDR1, VL CDR2, and VL CDR3 of 3-F03_411 (see Table 3, e.g., according to the IMGT definition, i.e., the amino acid sequences set forth in SEQ ID NOs: 37-39, respectively), wherein the light chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of 3-F03 (i.e., the amino acid sequence set forth in SEQ ID NO:31). In some embodiments, the anti-CD73 antibodies comprise: (i) a heavy chain comprising the a VH comprising the VH CDR1, VH CDR2, and VH CDR3 of 3-F03_413 (see Table 5, e.g., according to the IMGT definition, i.e., the amino acid sequences set forth in SEQ ID NOs: 34, 40, and 36, respectively), wherein the heavy chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain of 3-F03 (i.e., the amino acid sequence set forth in SEQ ID NO:33), and (ii) a light chain comprising a VL comprising the VL CDR1, VL CDR2, and VL CDR3 of 3-F03_413 (see Table 5, e.g., according to the IMGT definition, i.e., the amino acid sequences set forth in SEQ ID NOs: 37-39, respectively), wherein the light chain comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain of 3-F03_413 (i.e., the amino acid sequence set forth in SEQ ID NO:31). In some embodiments, the anti-CD73 antibody comprises: (i) a heavy chain comprising the amino acid sequence set forth in SEQ ID NO:30; and (ii) a light chain comprising the amino acid sequence set forth in SEQ ID NO:31. In some embodiments, the anti-CD73 antibody comprises: (i) a heavy chain comprising the amino acid sequence set forth in SEQ ID NO:33; and (ii) a light chain comprising the amino acid sequence set forth in SEQ ID NO:31.

The CD73-binding epitope of 3-F03 (and variants thereof, e.g., 3-F03_411 and 3-F03_413) contains AAVLPFGGTFDLVQ (SEQ ID NO:78) (i.e., amino acids 386-399 of the amino acid sequence set forth in SEQ ID NO:70) and ILPNFLANGGDGFQMIKDEL (SEQ ID NO:79) (i.e., amino acids 470-489 of the amino acid sequence set forth in SEQ ID NO:70). This disclosure features antibodies that bind to CD73 an epitope within AAVLPFGGTFDLVQ (SEQ ID NO:78) and ILPNFLANGGDGFQMIKDEL (SEQ ID NO:79). This disclosure features antibodies that bind to the same epitope as 3-F03 (or a variant thereof, e.g., 3-F03_411 or 3-F03_413). This disclosure also features antibodies that competitively inhibit binding of 3-F03 (or a variant thereof, e.g., 3-F03_411 or 3-F03_413) to human CD73.

In some embodiments, the VH of 3-F03 (or a variant thereof, e.g., 3-F03_411 or 3-F03_413) is linked to a heavy chain constant region comprising a CH1 domain and a hinge region. In some embodiments, the VH of 3-F03 (or a variant thereof, e.g., 3-F03_411 or 3-F03_413) is linked to a heavy chain constant region comprising a CH3 domain. In some embodiments, the CH3 domain lacks the C-terminal lysine (K) amino acid residue. In some embodiments, the CH3 domain contains the C-terminal lysine (K) amino acid residue. In certain embodiments, the VH of 3-F03 (or a variant thereof, e.g., 3-F03_411 or 3-F03_413) is linked to a heavy chain constant region comprising a CH1 domain, hinge region, CH2 domain, and CH3 domain from human IgG1. In some embodiments, the CH3 domain from human IgG1 lacks the C-terminal lysine (K) amino acid residue. In some embodiments, the CH3 domain from human IgG1 contains the C-terminal lysine (K) amino acid residue. In certain embodiments such an antibody contains one or more additional mutations in the heavy chain constant region that increase the stability of the antibody. In certain embodiments, the heavy chain constant region includes substitutions that modify the properties of the antibody (e.g., decrease Fc receptor binding, increase or decrease antibody glycosylation, decrease binding to C1q). In certain embodiments, the heavy chain constant region includes an alanine (A) at position Asparagine-297 (N297, according to EU numbering) of the heavy chain constant region to reduce effector function.

In certain embodiments, the anti-CD73 antibody is an IgG antibody. In one embodiment, the antibody is an IgG1 antibody. In one embodiment, the antibody is an IgG4 antibody. In another embodiment, the antibody is an IgG2 antibody. In certain embodiments, the anti-CD73 antibody comprises a heavy chain constant region lacking one or more lysine (K) amino acid residues relative to a wild type heavy chain constant region. For example, in certain embodiments, the antibody comprises heavy chain constant region lacking the C-terminal lysine (K) amino acid residue of the CH3 domain of the heavy chain constant region. In certain embodiments, the anti-CD73 antibody comprises a heavy chain constant region having the amino acid sequence set forth in SEQ ID NO:73. In certain embodiments, the anti-CD73 antibody comprises a heavy chain constant region having the amino acid sequence set forth in SEQ ID NO:75. In certain embodiments, the anti-CD73 antibody comprises a light chain constant region having the amino acid sequence set forth in SEQ ID NO:74. In certain embodiments, the anti-CD73 antibody comprises a heavy chain constant region having the amino acid sequence set forth in SEQ ID NO:73 and a light chain constant region having the amino acid sequence set forth in SEQ ID NO:74. In certain embodiments, the anti-CD73 antibody comprises a heavy chain constant region having the amino acid sequence set forth in SEQ ID NO:75 and a light chain constant region having the amino acid sequence set forth in SEQ ID NO:74.

### Antibody Fragments

In some instances, the anti-CD73 antibody is an antibody fragment. Fragments of the antibodies described herein (e.g., Fab, Fab′, F(ab′)₂, Facb, and Fv) may be prepared by proteolytic digestion of intact antibodies. For example, antibody fragments can be obtained by treating the whole antibody with an enzyme such as papain, pepsin, or plasmin. Papain digestion of whole antibodies produces F(ab)₂ or Fab fragments; pepsin digestion of whole antibodies yields F(ab′)₂ or Fab′; and plasmin digestion of whole antibodies yields Facb fragments.

Alternatively, antibody fragments can be produced recombinantly. For example, nucleic acids encoding the antibody fragments of interest can be constructed, introduced into an expression vector, and expressed in suitable host cells. See, e.g., Co, M. S. et al., J. Immunol., 152:2968-2976 (1994); Better, M. and Horwitz, A. H., Methods in Enzymology, 178:476-496 (1989); Plueckthun, A. and Skerra, A., Methods in Enzymology, 178:476-496 (1989); Lamoyi, E., Methods in Enzymology, 121:652-663 (1989); Rousseaux, J. et al., Methods in Enzymology, (1989) 121:663-669 (1989); and Bird, R. E. et al., TIBTECH, 9:132-137 (1991)). Antibody fragments can be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab)₂ fragments (Carter et al., Bio/Technology, 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046.

### Minibodies

In some instances, the anti-CD73 antibody is a minibody. Minibodies of anti-CD73 antibodies include diabodies, single chain (scFv), and single-chain (Fv)₂ (sc(Fv)₂).

A “diabody” is a bivalent minibody constructed by gene fusion (see, e.g., Holliger, P. et al., Proc. Natl. Acad. Sci. U.S.A, 90:6444-6448 (1993); EP 404,097; WO 93/11161). Diabodies are dimers composed of two polypeptide chains. The VL and VH domain of each polypeptide chain of the diabody are bound by linkers. The number of amino acid residues that constitute a linker can be between 2 to 12 residues (e.g., 3-10 residues or five or about five residues). The linkers of the polypeptides in a diabody are typically too short to allow the VL and VH to bind to each other. Thus, the VL and VH encoded in the same polypeptide chain cannot form a single-chain variable region fragment, but instead form a dimer with a different single-chain variable region fragment. As a result, a diabody has two antigen-binding sites.

An scFv is a single-chain polypeptide antibody obtained by linking the VH and VL with a linker (see, e.g., Huston et al., Proc. Natl. Acad. Sci. U.S.A, 85:5879-5883 (1988); and Plickthun, “The Pharmacology of Monoclonal Antibodies” Vol. 113, Ed Resenburg and Moore, Springer Verlag, New York, pp. 269-315, (1994)). The order of VHs and VLs to be linked is not particularly limited, and they may be arranged in any order. Examples of arrangements include: [VH] linker [VL]; or [VL] linker [VH]. The heavy chain variable domain and light chain variable domain in an scFv may be derived from any anti-CD73 antibody described herein.

An sc(Fv)₂ is a minibody in which two VHs and two VLs are linked by a linker to form a single chain (Hudson, et al., J. Immunol. Methods, (1999) 231: 177-189 (1999)). An sc(Fv)₂ can be prepared, for example, by connecting scFvs with a linker. The sc(Fv)₂ of the present invention include antibodies preferably in which two VHs and two VLs are arranged in the order of: VH, VL, VH, and VL ([VH] linker [VL] linker [VH] linker [VL]), beginning from the N terminus of a single-chain polypeptide; however the order of the two VHs and two VLs is not limited to the above arrangement, and they may be arranged in any order.

### Bispecific Antibodies

In some instances, the anti-CD73 antibody is a bispecific antibody. Bispecific antibodies are antibodies that have binding specificities for at least two different epitopes. Exemplary bispecific antibodies may bind to two different epitopes of the CD73 protein. Other such antibodies may combine a CD73 binding site with a binding site for another protein. Bispecific antibodies can be prepared as full length antibodies or low molecular weight forms thereof (e.g., F(ab′)₂ bispecific antibodies, sc(Fv)₂ bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

### Multivalent Antibodies

In some instances, the anti-CD73 antibody is a multivalent antibody. A multivalent antibody may be internalized (and/or catabolized) faster than a bivalent antibody by a cell expressing an antigen to which the antibodies bind. The antibodies describe herein can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. An exemplary dimerization domain comprises (or consists of) an Fc region or a hinge region. A multivalent antibody can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is a polypeptide chain of an Fc region, X1 and X2 represent an amino acid or peptide spacer, and n is 0 or 1.

### Conjugated Antibodies

In some instances, the anti-CD73 antibody is a conjugated antibody. The antibodies disclosed herein may be conjugated antibodies, which are bound to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, radioactive materials (e.g. ⁹⁰Y ¹³¹I), fluorescent substances, luminescent substances, haptens, enzymes, metal chelates, drugs, and toxins (e.g., calicheamicin, Pseudomonas exotoxin A, ricin (e.g. deglycosylated ricin A chain)).

In one embodiment, to improve the cytotoxic actions of anti-CD73 antibodies and consequently their therapeutic effectiveness, the antibodies are conjugated with highly toxic substances, including radioisotopes and cytotoxic agents. These conjugates can deliver a toxic load selectively to the target site (i.e., cells expressing the antigen recognized by the antibody) while cells that are not recognized by the antibody are spared. In order to minimize toxicity, conjugates are generally engineered based on molecules with a short serum half-life (thus, the use of murine sequences, and IgG3 or IgG4 isotypes).

In certain embodiments, an anti-CD73 antibody is modified with a moiety that improves its stabilization and/or retention in circulation, e.g., in blood, serum, or other tissues, e.g., by at least 1.5, 2, 5, 10, or 50 fold. For example, the anti-CD73 antibody can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the anti-CD73 antibody can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

The above-described conjugated antibodies can be prepared by performing chemical modifications on the antibodies, respectively, or the lower molecular weight forms thereof described herein. Methods for modifying antibodies are well known in the art (e.g., U.S. Pat. Nos. 5,057,313 and 5,156,840).

### Methods of Producing Antibodies

Antibodies may be produced in bacterial or eukaryotic cells. Some antibodies, e.g., Fabs, can be produced in bacterial cells, e.g., E. coli cells. Antibodies can also be produced in eukaryotic cells such as transformed cell lines (e.g., CHO, 293E, COS). In addition, antibodies (e.g., scFvs) can be expressed in a yeast cell such as Pichia (see, e.g., Powers et al., J Immunol Methods. 251:123-35 (2001)), Hansenula, or Saccharomyces. To produce the antibody of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5a, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr—CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601 621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of an anti-CD73 antibody (e.g., CL25, HzCL25, 3-F03, 3-F03_411, or 3-F03_413) is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Polynucleotides, Expression Vectors, and Cells

The disclosure also provides polynucleotides and vectors encoding an anti-CD73 antibody or portion thereof (e.g., VH, VL, HC, or LC) described herein. The polynucleotides of the disclosure can be in the form of RNA or in the form of DNA. In some instances, the polynucleotide is DNA. In some instances, the polynucleotide is complementary DNA (cDNA). In some instances, the polynucleotide is RNA.

In some instances, the polynucleotide encodes a VH comprising the VH CDR1, VH CDR2, and VH CDR3 of any antibody described herein (see, e.g., Tables 1, 2, 3, and 5). In some instances, the polynucleotide encodes a VL comprising the VL CDR1, VL CDR2, and VL CDR3 of any antibody described herein (see, e.g., Tables 1, 2, 3, and 5). In some instances, the polynucleotide encodes a heavy chain comprising a VH comprising the VH CDR1, VH CDR2, and VH CDR3 of any antibody described herein (see, e.g., Tables 1, 2, 3, and 5). In some instances, the polynucleotide encodes a light chain comprising a VL comprising the VL CDR1, VL CDR2, and VL CDR3 of any antibody described herein (see, e.g., Tables 1, 2, 3, and 5). In some instances, the polynucleotide is operably linked to a promoter.

In some instances, the polynucleotide comprises: (i) a first nucleic acid sequence encoding a first polypeptide, wherein the first polypeptide comprises a VH comprising the VH CDR1, VH CDR2, and VH CDR3 of any antibody described herein (see, e.g., Tables 1, 2, 3, and 5); and (ii) a second nucleic acid sequence encoding a second polypeptide, wherein the second polypeptide comprises a VL comprising the VL CDR1, VL CDR2, and VL CDR3 of any antibody described herein (see, e.g., Tables 1, 2, 3, and 5). In some instances, the polynucleotide comprises: (i) a first nucleic acid sequence encoding a first polypeptide, wherein the first polypeptide comprises a heavy chain comprising a VH comprising the VH CDR1, VH CDR2, and VH CDR3 of any antibody described herein (see, e.g., Tables 1, 2, 3, and 5); and (ii) a second nucleic acid sequence encoding a second polypeptide, wherein the second polypeptide comprises a light chain comprising a VL comprising the VL CDR1, VL CDR2, and VL CDR3 of any antibody described herein (see, e.g., Tables 1, 2, 3, and 5). In some instances, the first nucleic acid is operably linked to a first promoter and the second nucleic acid is operably linked to a second promoter.

In some instances, the polynucleotide encodes the VH of CL25 or a variant thereof (e.g., a humanized version thereof, e.g., HzCL25). In some instances, the polynucleotide encodes a polypeptide comprising an amino acid sequence having at least 80%, at least 85%, at least 90%, at least 95%, at least 97%, or 100% identity to the amino acid sequence set forth in any one of SEQ ID NOs:22, 26, and 82-84. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:22, 26, and 82-84. In some instances, the polynucleotide encodes a polypeptide comprising the amino acid sequence set forth in any one of SEQ ID NOs:22, 26, and 82-84. In some instances, the polynucleotide encodes a polypeptide comprising the amino acid sequence set forth in SEQ ID NO:22. In some instances, the polynucleotide is operably linked to a promoter.

In some instances, the polynucleotide encodes the VL of CL25 or a variant thereof (e.g., a humanized version thereof, e.g., HzCL25). In some instances, the polynucleotide encodes a polypeptide comprising an amino acid sequence having at least 80%, at least 85%, at least 90%, at least 95%, at least 97%, or 100% identity to the amino acid sequence set forth in any one of SEQ ID NOs:23, 27, 80, and 81. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NO:23, 27, 80, and 81. In some instances, the polynucleotide encodes a polypeptide comprising the amino acid sequence set forth in any one of SEQ ID NOs:23, 27, 80, and 81. In some instances, the polynucleotide encodes a polypeptide comprising the amino acid sequence set forth in SEQ ID NO:23. In some instances, the polynucleotide is operably linked to a promoter.

In some instances, the polynucleotide comprises: (i) a first nucleic acid encoding a first polypeptide, wherein the first polypeptide comprises the VH of CL25 or a variant thereof (e.g., a humanized version thereof, e.g., HzCL25); and (ii) a second nucleic acid encoding a second polypeptide, wherein the second polypeptide comprises the VL of CL25 or a variant thereof (e.g., a humanized version thereof, e.g., HzCL25). In some instances, the polynucleotide comprises: (i) a first nucleic acid sequence encoding a first polypeptide, wherein the first polypeptide comprises an amino acid sequence having at least 80%, at least 85%, at least 90%, at least 95%, at least 97%, or 100% identity to the amino acid sequence set forth in any one of SEQ ID NOs:22, 26, and 82-84, and (ii) a second nucleic acid sequence encoding a second polypeptide, wherein the second polypeptide comprises an amino acid sequence having at least 80%, at least 85%, at least 90%, at least 95%, at least 97%, or 100% identity to the amino acid sequence set forth in any one of SEQ ID NOs:23, 27, 80, and 81. In some instances, the polynucleotide comprises: (i) a first nucleic acid sequence encoding a first polypeptide, wherein the first polypeptide comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:22, 26, and 82-84; and (ii) a second nucleic acid sequence encoding a second polypeptide, wherein the second polypeptide comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:23, 27, 80, and 81. In some instances, the first nucleic acid encodes the amino acid sequence set forth in any one of SEQ ID NOs:22, 26, and 82-84 and the second nucleic acid encodes the amino acid sequence set forth in any one of SEQ ID NOs:23, 27, 80, and 81. In some instances, the first nucleic acid encodes the amino acid sequence set forth in SEQ ID NO:22 and the second nucleic acid encodes the amino acid sequence set forth in SEQ ID NO:23. In some instances, the first nucleic acid is operably linked to a first promoter and the second nucleic acid is operably linked to a second promoter.

In some instances, the polynucleotide encodes the heavy chain of CL25 or a variant thereof (e.g., a humanized version thereof, e.g., HzCL25). In some instances, the polynucleotide encodes a polypeptide comprising an amino acid sequence having at least 80%, at least 85%, at least 90%, at least 95%, at least 97%, or 100% identity to the amino acid sequence set forth in SEQ ID NO:24. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in SEQ ID NO:24. In some instances, the polynucleotide encodes a polypeptide comprising the amino acid sequence set forth in SEQ ID NO:24. In some instances, the polynucleotide comprises the sequence set forth in SEQ ID NO:89. In some instances, the polynucleotide is operably linked to a promoter.

In some instances, the polynucleotide encodes the light chain of CL25 or a variant thereof (e.g., a humanized version thereof, e.g., HzCL25). In some instances, the polynucleotide encodes a polypeptide comprising an amino acid sequence having at least 80%, at least 85%, at least 90%, at least 95%, at least 97%, or 100% identity to the amino acid sequence set forth in SEQ ID NO:25. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in SEQ ID NO:25. In some instances, the polynucleotide encodes a polypeptide comprising the amino acid sequence set forth in SEQ ID NO:25. In some instances, the polynucleotide comprises the sequence set forth in SEQ ID NO:90. In some instances, the polynucleotide is operably linked to a promoter.

In some instances, the polynucleotide encodes the heavy chain of CL25 or a variant thereof (e.g., a humanized version thereof, e.g., HzCL25) and the light chain of CL25 or a variant thereof (e.g., a humanized version thereof, e.g., HzCL25). In some instances, the polynucleotide comprises: (i) a first nucleic acid encoding a first polypeptide, wherein the first polypeptide comprises an amino acid sequence having at least 80%, at least 85%, at least 90%, at least 95%, at least 97%, or 100% identity to the amino acid sequence set forth in SEQ ID NO:24, and (ii) a second nucleic acid encoding a second polypeptide, wherein the second polypeptide comprises an amino acid sequence having at least 80%, at least 85%, at least 90%, at least 95%, at least 97%, or 100% identity to the amino acid sequence set forth in SEQ ID NO:25. In some instances, the polynucleotide comprises: (i) a first nucleic acid encoding a first polypeptide, wherein the first polypeptide comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO:24, and (ii) a second nucleic acid encoding a second polypeptide, wherein the second polypeptide comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO:25. In some instances, the polynucleotide comprises: (i) a first nucleic acid encoding a first polypeptide, wherein the first polypeptide comprises the amino acid sequence set forth in SEQ ID NO:24, and (ii) a second nucleic acid encoding a second polypeptide, wherein the second polypeptide comprises the amino acid sequence set forth in SEQ ID NO:25. In some instances, the polynucleotide comprises: (i) a first nucleic acid comprising the sequence set forth in SEQ ID NO:89, and (ii) a second nucleic acid comprising the sequence set forth in SEQ ID NO:90. In some instances, the first nucleic acid is operably linked to a first promoter and the second nucleic acid is operably linked to a second promoter.

In some instances, the polynucleotide encodes the VH of 3-F03 or a variant thereof (e.g., 3-F03_411 or 3-F03_413). In some instances, the polynucleotide encodes a polypeptide comprising an amino acid sequence having at least 80%, at least 85%, at least 90%, at least 95%, at least 97%, or 100% identity to the amino acid sequence set forth in any one of SEQ ID NOs: 32, 60, 62, 63, 67-69, 77, and 85-88. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 32, 60, 62, 63, 67-69, 77, and 85-88. In some instances, the polynucleotide encodes a polypeptide comprising the amino acid sequence set forth in any one of SEQ ID NOs: 32, 60, 62, 63, 67-69, 77, and 85-88. In some instances, the polynucleotide encodes a polypeptide comprising the amino acid sequence set forth in SEQ ID NO:62. In some instances, the polynucleotide encodes a polypeptide comprising the amino acid sequence set forth in SEQ ID NO:63. In some instances, the polynucleotide is operably linked to a promoter.

In some instances, the polynucleotide encodes the VL of 3-F03 or a variant thereof (e.g., 3-F03_411 or 3-F03_413). In some instances, the polynucleotide encodes a polypeptide comprising an amino acid sequence having at least 80%, at least 85%, at least 90%, at least 95%, at least 97%, or 100% identity to the amino acid sequence set forth in any one of SEQ ID NOs:61, 64, and 65. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NO: 61, 64, and 65. In some instances, the polynucleotide encodes a polypeptide comprising the amino acid sequence set forth in any one of SEQ ID NOs: 61, 64, and 65. In some instances, the polynucleotide encodes a polypeptide comprising the amino acid sequence set forth in SEQ ID NO:61. In some instances, the polynucleotide is operably linked to a promoter.

In some instances, the polynucleotide comprises: (i) a first nucleic acid encoding a first polypeptide, wherein the first polypeptide comprises the VH of 3-F03 or a variant thereof (e.g., 3-F03_411 or 3-F03_413); and (ii) a second nucleic acid encoding a second polypeptide, wherein the second polypeptide comprises the VL of 3-F03 or a variant thereof (e.g., 3-F03_411 or 3-F03_413). In some instances, the polynucleotide comprises: (i) a first nucleic acid sequence encoding a first polypeptide, wherein the first polypeptide comprises an amino acid sequence having at least 80%, at least 85%, at least 90%, at least 95%, at least 97%, or 100% identity to the amino acid sequence set forth in any one of SEQ ID NOs: 32, 60, 62, 63, 67-69, 77, and 85-88, and (ii) a second nucleic acid sequence encoding a second polypeptide, wherein the second polypeptide comprises an amino acid sequence having at least 80%, at least 85%, at least 90%, at least 95%, at least 97%, or 100% identity to the amino acid sequence set forth in any one of SEQ ID NOs:61, 64, and 65. In some instances, the polynucleotide comprises: (i) a first nucleic acid sequence encoding a first polypeptide, wherein the first polypeptide comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 32, 60, 62, 63, 67-69, 77, and 85-88; and (ii) a second nucleic acid sequence encoding a second polypeptide, wherein the second polypeptide comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:61, 64, and 65. In some instances, the first nucleic acid encodes the amino acid sequence set forth in any one of SEQ ID NOs: 32, 60, 62, 63, 67-69, 77, and 85-88 and the second nucleic acid encodes the amino acid sequence set forth in any one of SEQ ID NOs:61, 64, and 65. In some instances, the first nucleic acid encodes the amino acid sequence set forth in SEQ ID NO:62 and the second nucleic acid encodes the amino acid sequence set forth in SEQ ID NO:61. In some instances, the first nucleic acid encodes the amino acid sequence set forth in SEQ ID NO:63 and the second nucleic acid encodes the amino acid sequence set forth in SEQ ID NO:61. In some instances, the first nucleic acid is operably linked to a first promoter and the second nucleic acid is operably linked to a second promoter.

In some instances, the polynucleotide encodes the heavy chain of 3-F03 or a variant thereof (e.g., 3-F03_411 or 3-F03_413). In some instances, the polynucleotide encodes a polypeptide comprising an amino acid sequence having at least 80%, at least 85%, at least 90%, at least 95%, at least 97%, or 100% identity to the amino acid sequence set forth in any one of SEQ ID NO: 30, 33, and 66. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NO: 30, 33, and 66. In some instances, the polynucleotide encodes a polypeptide comprising the amino acid sequence set forth in any one of SEQ ID NO: 30, 33, and 66. In some instances, the polynucleotide encodes a polypeptide comprising the amino acid sequence set forth in SEQ ID NO:30. In some instances, the polynucleotide encodes a polypeptide comprising the amino acid sequence set forth in SEQ ID NO:33. In some instances, the polynucleotide comprises the sequence set forth in SEQ ID NO:91. In some instances, the polynucleotide comprises the sequence set forth in SEQ ID NO:93. In some instances, the polynucleotide is operably linked to a promoter.

In some instances, the polynucleotide encodes the light chain of 3-F03 or a variant thereof (e.g., 3-F03_411 or 3-F03_413). In some instances, the polynucleotide encodes a polypeptide comprising an amino acid sequence having at least 80%, at least 85%, at least 90%, at least 95%, at least 97%, or 100% identity to the amino acid sequence set forth in SEQ ID NO:31. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in SEQ ID NO: 31. In some instances, the polynucleotide encodes a polypeptide comprising the amino acid sequence set forth in SEQ ID NO: 31. In some instances, the polynucleotide encodes a polypeptide comprising the amino acid sequence set forth in SEQ ID NO:31. In some instances, the polynucleotide comprises the sequence set forth in SEQ ID NO:92. In some instances, the polynucleotide is operably linked to a promoter.

In some instances, the polynucleotide encodes the heavy chain of 3-F03 or a variant thereof (e.g., 3-F03_411 or 3-F03_413) and the light chain of 3-F03 or a variant thereof (e.g., 3-F03_411 or 3-F03_413). In some instances, the polynucleotide comprises: (i) a first nucleic acid encoding a first polypeptide, wherein the first polypeptide comprises an amino acid sequence having at least 80%, at least 85%, at least 90%, at least 95%, at least 97%, or 100% identity to the amino acid sequence set forth in any one of SEQ ID NO: 30, 33, and 66, and (ii) a second nucleic acid encoding a second polypeptide, wherein the second polypeptide comprises an amino acid sequence having at least 80%, at least 85%, at least 90%, at least 95%, at least 97%, or 100% identity to the amino acid sequence set forth in SEQ ID NO: 31. In some instances, the polynucleotide comprises: (i) a first nucleic acid encoding a first polypeptide, wherein the first polypeptide comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NO: 30, 33, and 66, and (ii) a second nucleic acid encoding a second polypeptide, wherein the second polypeptide comprises an amino acid sequence having one or more (e.g., 1, 2, 3, 4, 5, 6, 7, 8, 9, 10) amino acid substitutions, additions, and/or deletions relative to the amino acid sequence set forth in SEQ ID NO: 31. In some instances, the polynucleotide comprises: (i) a first nucleic acid encoding a first polypeptide, wherein the first polypeptide comprises the amino acid sequence set forth in any one of SEQ ID NO: 30, 33, and 66, and (ii) a second nucleic acid encoding a second polypeptide, wherein the second polypeptide comprises the amino acid sequence set forth in SEQ ID NO: 31. In some instances, the polynucleotide comprises: (i) a first nucleic acid encoding a first polypeptide, wherein the first polypeptide comprises the amino acid sequence set forth in SEQ ID NO:30, and (ii) a second nucleic acid encoding a second polypeptide, wherein the second polypeptide comprises the amino acid sequence set forth in SEQ ID NO:31. In some instances, the polynucleotide comprises: (i) a first nucleic acid encoding a first polypeptide, wherein the first polypeptide comprises the amino acid sequence set forth in SEQ ID NO:33, and (ii) a second nucleic acid encoding a second polypeptide, wherein the second polypeptide comprises the amino acid sequence set forth in SEQ ID NO:31. In some instances, the polynucleotide comprises: (i) a first nucleic acid comprising the sequence set forth in SEQ ID NO:91, and (ii) a second nucleic acid comprising the sequence set forth in SEQ ID NO:92. In some instances, the polynucleotide comprises: (i) a first nucleic acid comprising the sequence set forth in SEQ ID NO:93, and (ii) a second nucleic acid comprising the sequence set forth in SEQ ID NO:92. In some instances, the first nucleic acid is operably linked to a first promoter and the second nucleic acid is operably linked to a second promoter.

In some embodiments, a polynucleotide described herein is isolated.

Also provided herein are expression vectors encoding the anti-CD73 antibodies or portions thereof (e.g., VH, VL, HC, and/or LC) described herein. Also provided herein are expression vectors comprising one or more polynucleotides described herein. Various types of expression vectors are known in the art and described herein (e.g., see the section “Methods of Producing Antibodies” above).

Also provided herein are cells comprising the anti-CD73 antibodies described herein. Also provided herein are cells comprising one or more polynucleotides described herein. Also provided herein are cells comprising one or more expression vectors described herein. Various types of cells are known in the art and described herein (e.g., see the section “Methods of Producing Antibodies” above).

Anti-CD73 Antibodies with Altered Glycosylation

Different glycoforms can profoundly affect the properties of a therapeutic, including pharmacokinetics, pharmacodynamics, receptor-interaction and tissue-specific targeting (Graddis et al., 2002, Curr Pharm Biotechnol. 3: 285-297). In particular, for antibodies, the oligosaccharide structure can affect properties relevant to protease resistance, the serum half-life of the antibody mediated by the FcRn receptor, phagocytosis and antibody feedback, in addition to effector functions of the antibody (e.g., binding to the complement complex C1, which induces CDC, and binding to FcγR receptors, which are responsible for modulating the ADCC pathway) (Nose and Wigzell, 1983; Leatherbarrow and Dwek, 1983; Leatherbarrow et al., 1985; Walker et al., 1989; Carter et al., 1992, PNAS, 89: 4285-4289).

Accordingly, another means of modulating effector function of antibodies includes altering glycosylation of the antibody constant region. Altered glycosylation includes, for example, a decrease or increase in the number of glycosylated residues, a change in the pattern or location of glycosylated residues, as well as a change in sugar structure(s). The oligosaccharides found on human IgGs affects their degree of effector function (Raju, T. S. BioProcess International April 2003. 44-53); the microheterogeneity of human IgG oligosaccharides can affect biological functions such as CDC and ADCC, binding to various Fc receptors, and binding to C1q protein (Wright A. & Morrison SL. TIBTECH 1997, 15 26-32; Shields et al. J Biol Chem. 2001 276(9):6591-604; Shields et al. J Biol Chem. 2002; 277(30):26733-40; Shinkawa et al. J Biol Chem. 2003 278(5):3466-73; Umana et al. Nat Biotechnol. 1999 February; 17(2): 176-80). For example, the ability of IgG to bind C1q and activate the complement cascade may depend on the presence, absence or modification of the carbohydrate moiety positioned between the two CH2 domains (which is normally anchored at Asn297) (Ward and Ghetie, Therapeutic Immunology 2:77-94 (1995). Thus, in some instances, the anti-CD73 antibody contains an Asn297Ala substitution relative to a wild type constant region.

Glycosylation sites in an Fc-containing polypeptide, for example an antibody such as an IgG antibody, may be identified by standard techniques. The identification of the glycosylation site can be experimental or based on sequence analysis or modeling data. Consensus motifs, that is, the amino acid sequence recognized by various glycosyl transferases, have been described. For example, the consensus motif for an N-linked glycosylation motif is frequently NXT or NXS, where X can be any amino acid except proline. Several algorithms for locating a potential glycosylation motif have also been described. Accordingly, to identify potential glycosylation sites within an antibody or Fc-containing fragment, the sequence of the antibody is examined, for example, by using publicly available databases such as the website provided by the Center for Biological Sequence Analysis (see NetNGlyc services for predicting N-linked glycosylation sites and NetOGlyc services for predicting O-linked glycosylation sites).

In vivo studies have confirmed the reduction in the effector function of a glycosyl antibodies. For example, an a glycosyl anti-CD8 antibody is incapable of depleting CD8-bearing cells in mice (Isaacs, 1992 J. Immunol. 148: 3062) and an a glycosyl anti-CD3 antibody does not induce cytokine release syndrome in mice or humans (Boyd, 1995 supra; Friend, 1999 Transplantation 68:1632). Aglycosylated forms of the anti-CD73 antibody also have reduced effector function.

Importantly, while removal of the glycans in the CH2 domain appears to have a significant effect on effector function, other functional and physical properties of the antibody remain unaltered. Specifically, it has been shown that removal of the glycans had little to no effect on serum half-life and binding to antigen (Nose, 1983 supra; Tao, 1989 supra; Dorai, 1991 supra; Hand, 1992 supra; Hobbs, 1992 Mol. Immunol. 29:949).

The anti-CD73 antibodies of the present invention may be modified or altered to elicit increased or decreased effector function(s) (compared to a second CD73-specific antibody). Methods for altering glycosylation sites of antibodies are described, e.g., in U.S. Pat. Nos. 6,350,861 and 5,714,350, WO 05/18572 and WO 05/03175; these methods can be used to produce anti-CD73 antibodies of the present invention with altered, reduced, or no glycosylation.

### Indications

The anti-CD73 antibodies of the present disclosure can modulate the activity of CD73. Accordingly, the compounds, salts or stereoisomers described herein can be used in methods of inhibiting CD73 by contacting CD73 with any one or more of the antibodies or compositions described herein. In some embodiments, the antibodies can be used in methods of inhibiting activity of CD73 in an individual/patient in need of the inhibition by administering an effective amount of an antibody described herein. In some embodiments, modulating is inhibiting. In some embodiments, the contacting is in vivo. In some embodiments, the contacting is ex vivo or in vitro.

Another aspect of the present disclosure pertains to methods of treating a CD73-associated disease or disorder in an individual (e.g., patient) by administering to the individual in need of such treatment a therapeutically effective amount or dose of one or more antibodies of the present disclosure or a pharmaceutical composition thereof. A CD73-associated disease or disorder can include any disease, disorder or condition that is directly or indirectly linked to expression or activity of CD73, including overexpression and/or abnormal activity levels.

Another aspect of the present disclosure pertains to methods of treating a disease or disorder (e.g., cancer) in an individual (e.g., patient) by administering to the individual in need of such treatment a therapeutically effective amount or dose of one or more antibodies of the present disclosure or a pharmaceutical composition thereof, wherein the disease or disorder has a high adenosine signature. Methods of determining that a disease or disorder has a high adenosine signature are known in the art. For instance, gene expression analysis of tumor tissue may be performed using a defined panel of adenosine-responsive genes.

The compounds of the present disclosure are useful in the treatment of diseases related to the activity of CD73 including, for example, cancer, inflammatory diseases, cardiovascular diseases, neurodegenerative diseases, immunomodulatory disorders, central nerve system diseases, and diabetes.

Based on the compelling roles of CD73 in multiple immunosuppressive mechanisms, developing inhibitors can boost the immune system to suppress tumor progression. Anti-CD73 antibodies can be used to treat, alone or in combination with other therapies, bladder cancer, lung cancer (e.g., non-small cell lung cancer (NSCLC), lung metastasis), melanoma (e.g., metastatic melanoma), breast cancer, cervical cancer, ovarian cancer, colorectal cancer, pancreatic cancer, esophageal cancer, prostate cancer, kidney cancer, skin cancer, thyroid cancer, liver cancer (e.g., hepatocellular carcinoma), uterine cancer, head and neck cancer (e.g., head and neck squamous cell carcinoma), and renal cell carcinoma. In some embodiments, the prostate cancer is metastatic castrate-resistant prostate carcinoma (mCRPC). In some embodiments, the colorectal cancer is colorectal carcinoma (CRC).

In some embodiments, the disease or disorder is lung cancer (e.g., non-small cell lung cancer), melanoma, pancreatic cancer, breast cancer, head and neck squamous cell carcinoma, prostate cancer, liver cancer, color cancer, endometrial cancer, bladder cancer, skin cancer, cancer of the uterus, renal cancer, gastric cancer, or sarcoma. In some embodiments, the sarcoma is Askin's tumor, sarcoma botryoides, chondrosarcoma, Ewing's sarcoma, malignant hemangioendothelioma, malignant schwannoma, osteosarcoma, alveolar soft part sarcoma, angiosarcoma, cystosarcoma phyllodes, dermatofibrosarcoma protuberans, desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, malignant peripheral nerve sheath tumor (MPNST), neurofibrosarcoma, rhabdomyosarcoma, synovial sarcoma, or undifferentiated pleomorphic sarcoma.

In some embodiments, the disease or disorder is head and neck cancer (e.g., head and neck squamous cell carcinoma), colorectal cancer, lung cancer (e.g., non-small cell lung cancer (NSCLC)), melanoma, ovarian, bladder, liver cancer (e.g., hepatocellular carcinoma), or renal cell carcinoma.

In some embodiments, the disease or disorder is mesothelioma or adenocarcinoma. In some embodiments, the disease or disorder is mesothelioma. In some embodiments, the disease or disorder is adenocarcinoma.

MDSC (myeloid-derived suppressor cells) are a heterogenous group of immune cells from the myeloid lineage (a family of cells that originate from bone marrow stem cells). MDSCs strongly expand in pathological situations such as chronic infections and cancer, as a result of an altered haematopoiesis. MDSCs are discriminated from other myeloid cell types in which they possess strong immunosuppressive activities rather than immunostimulatory properties. Similar to other myeloid cells, MDSCs interact with other immune cell types including T cells, dendritic cells, macrophages and natural killer cells to regulate their functions. In some embodiments, the compounds, etc. described herein can be used in methods related to cancer tissue (e.g., tumors) with high infiltration of MDSCs, including Solid tumors with high basal level of macrophage and/or MDSC infiltration.

In some embodiments, the antibodies of the disclosure can be used in treating pulmonary inflammation, including bleomycin-induced pulmonary fibrosis and injury related to adenosine deaminase deficiency.

In some embodiments, the antibodies of the disclosure can be used as a treatment for inflammatory disease such as allergic reactions (e.g., CD73-dependent allergic reactions) and other CD73-immune reactions. Further inflammatory diseases that can be treated by antibodies of the disclosure include respiratory disorders, sepsis, reperfusion injury, and thrombosis.

In some embodiments, the antibodies of the disclosure can be used as a treatment for cardiovascular disease such as coronary artery disease (myocardial infarction, angina pectoris, heart failure), cerebrovascular disease (stroke, transient ischemic attack), peripheral artery disease, and aortic atherosclerosis and aneurysm. Atherosclerosis is an underlying etiologic factor in many types of cardiovascular disease. Atherosclerosis begins in adolescence with fatty streaks, which progress to plaques in adulthood and finally results in thrombotic events that cause occlusion of vessels leading to clinically significant morbidity and mortality.

In some embodiments, the antibodies of the disclosure can be used as a treatment for disorders in motor activity; deficiency caused by degeneration of the striatonigral dopamine system; and Parkinson's disease; some of the motivational symptoms of depression.

In some embodiments, the compounds of the disclosure can be used as a treatment for diabetes and related disorders, such as insulin resistance. Diabetes affects the production of adenosine and the expression of A2B adenosine receptors (A2BRs) that stimulate IL-6 and CRP production, insulin resistance, and the association between A2BR gene single-nucleotide polymorphisms (ADORA2B SNPs) and inflammatory markers. The increased A2BR signaling in diabetes may increase insulin resistance in part by elevating pro-inflammatory mediators. Selective anti-CD73 antibodies may be useful to treat insulin resistance.

The terms “individual” or “patient” or “subject”, used interchangeably, refer to any animal, including mammals, preferably mice, rats, other rodents, rabbits, dogs, cats, swine, cattle, sheep, horses, or primates, and most preferably humans (i.e., a human subject).

The phrase “therapeutically effective amount” refers to the amount of active antibody or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the antibodies of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Pharmaceutical Compositions

An anti-CD73 antibody described herein can be formulated as a pharmaceutical composition for administration to a subject, e.g., to treat a disorder described herein. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J. Pharm. Sci. 66:1-19).

Pharmaceutical formulation is a well-established art, and is further described, e.g., in Gennaro (ed.), Remington: The Science and Practice of Pharmacy, 20th ed., Lippincott, Williams & Wilkins (2000) (ISBN: 0683306472); Ansel et al., Pharmaceutical Dosage Forms and Drug Delivery Systems, 7th Ed., Lippincott Williams & Wilkins Publishers (1999) (ISBN: 0683305727); and Kibbe (ed.), Handbook of Pharmaceutical Excipients American Pharmaceutical Association, 3rd ed. (2000) (ISBN: 091733096X).

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the anti-CD73 antibody may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

### Administration

The anti-CD73 antibody can be administered to a subject, e.g., a subject in need thereof, for example, a human subject, by a variety of methods. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection. In some cases, administration can be oral.

The route and/or mode of administration of the antibody can also be tailored for the individual case, e.g., by monitoring the subject, e.g., using tomographic imaging, e.g., to visualize a tumor.

The antibody can be administered as a fixed dose, or in a mg/kg patient weight dose. The dose can also be chosen to reduce or avoid production of antibodies against the anti-CD73 antibody. Dosage regimens are adjusted to provide the desired response, e.g., a therapeutic response or a combinatorial therapeutic effect. Generally, doses of the anti-CD73 antibody (and optionally a second agent) can be used in order to provide a subject with the agent in bioavailable quantities.

Dosage unit form or “fixed dose” or “flat dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent. Single or multiple dosages may be given. Alternatively, or in addition, the antibody may be administered via continuous infusion.

The following are examples of the practice of the invention. They are not to be construed as limiting the scope of the invention in any way.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: Generation of Anti-Human CD73 Monoclonal Antibodies

To generate anti-human CD73 monoclonal antibodies, mice were immunized with recombinant human CD73 (SEQ ID NO:70) protein comprising a C-terminal HIS-tag and B cells were isolated from the mouse spleen and lymph nodes. Antibody sequences of the B cells were determined using 10× Genomics VH/VL paired B cell sequencing. The murine VH/VL pairs were expressed as chimeras with huIgG1 Fc (SEQ ID NOs: 73 and 74) and tested for binding and functionality. An antibody designated CL25 was produced by this process. Table 1, above, shows the amino acid sequences of the CL25 CDRs according to IMGT, Chothia, AbM, Kabat, and Contact numbering and the mature VH, VL, heavy chain, and light chain.

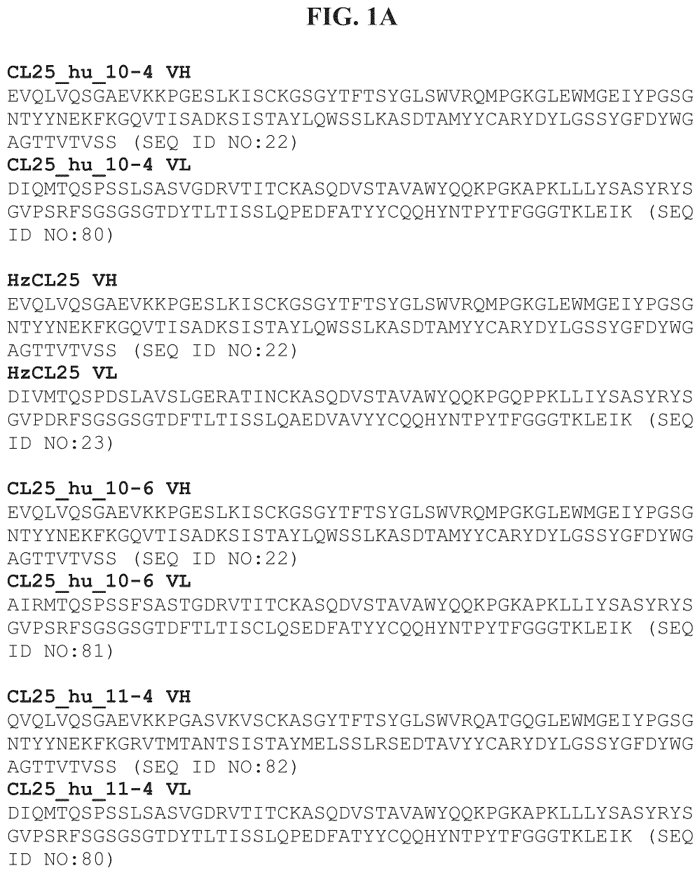

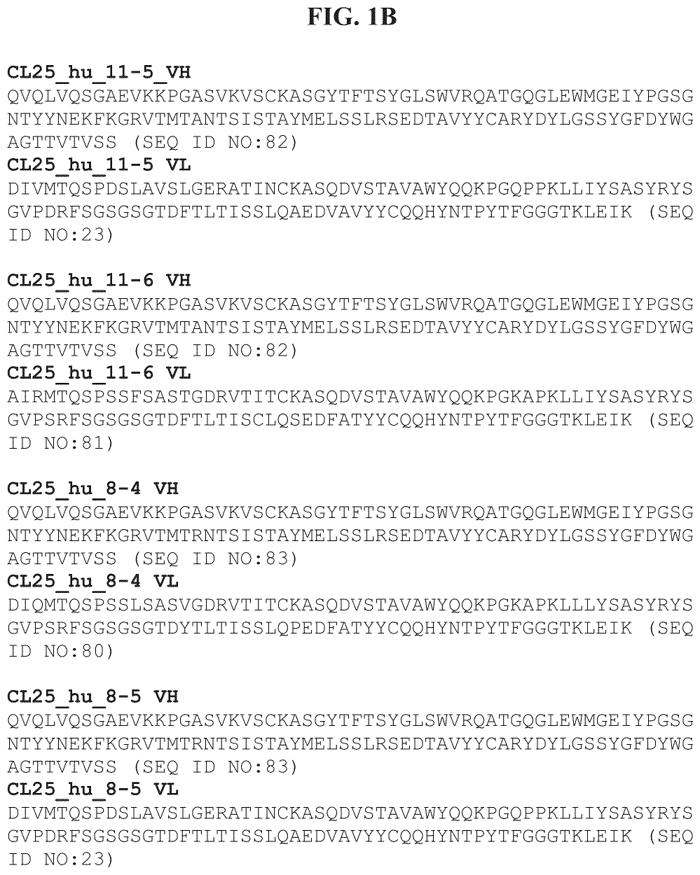

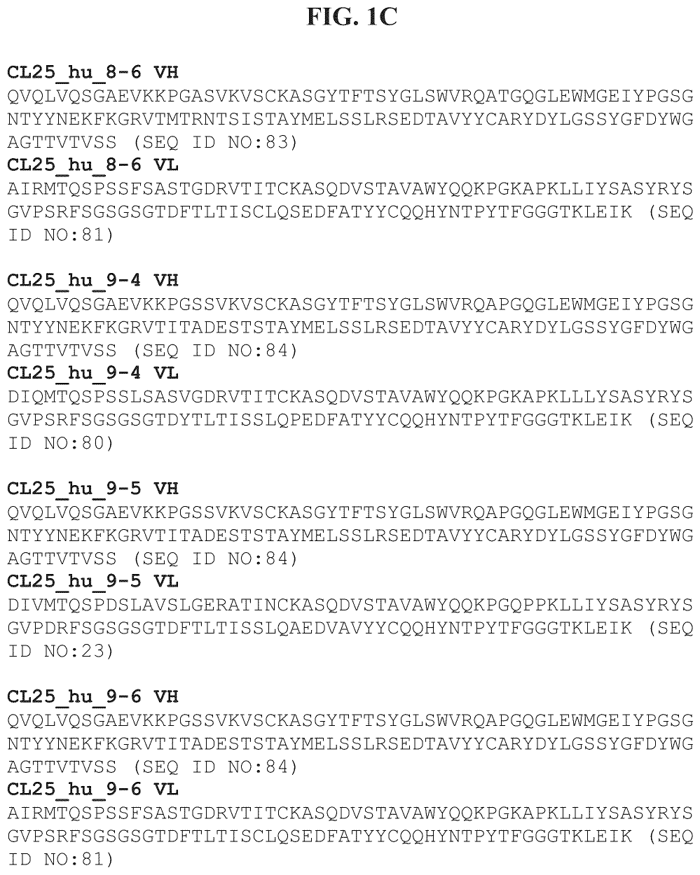

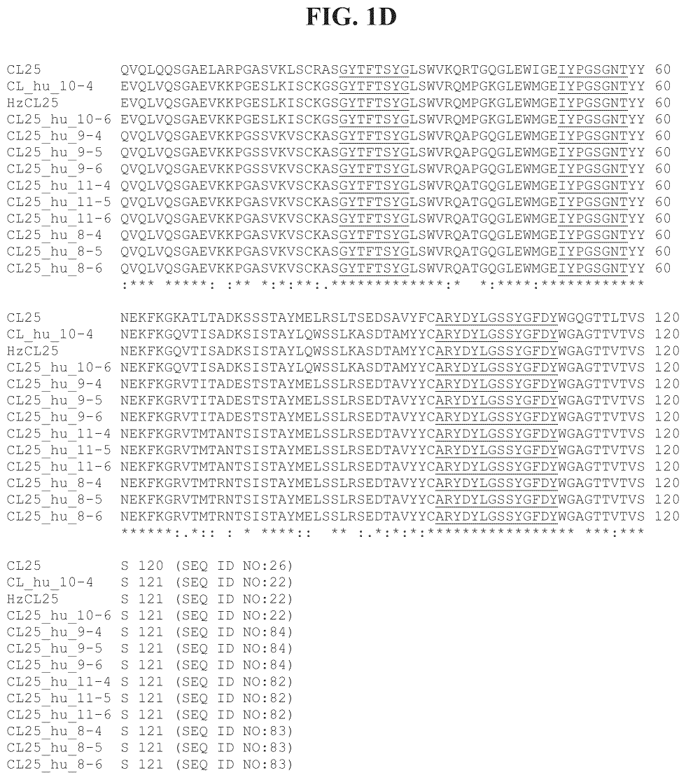

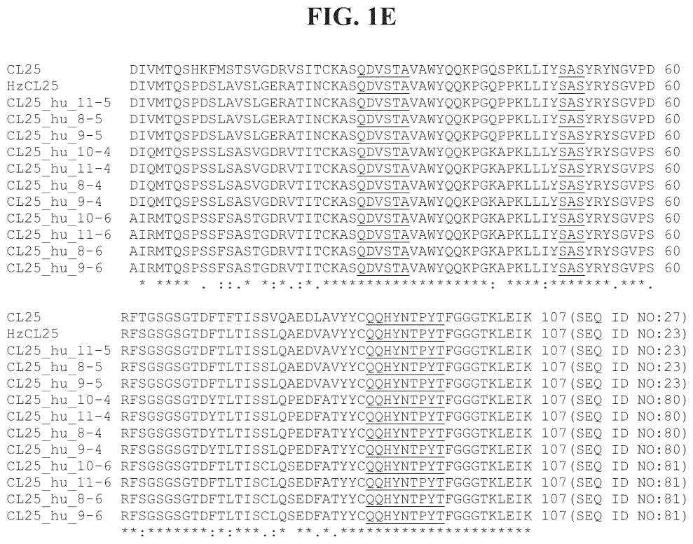

Chimeric antibody CL25 (comprising the murine VH of SEQ ID NO:26 and murine VL of SEQ ID NO:27) was humanized to minimize the immunogenicity of the antibody frameworks while maintaining specific activity. Humanization was conducted by aligning the VH and VL sequences to a database of human VH and VK genes. The CDRs (Table 1) from the murine CL25 antibody were grafted into several top human VH and VK genes. The VH and VL sequences of exemplary humanized CL25 antibodies are depicted in FIG. 1A-FIG. 1C. Alignments of the VH and VL of CL25 and exemplary humanized CL25 antibodies are depicted in FIG. 1D and FIG. 1E, respectively. Several framework mutations present in the murine CL25 were also tested along with the murine CDRs (FIG. 1A-FIG. 1E). The humanized version of CL25 having a VH of SEQ ID NO:22 and a VL of SEQ ID NO:23, referred to herein as “HzCL25”, was selected for further studies. Table 2, above, shows the amino acid sequences of the HzCL25 CDRs according to IMGT, Chothia, AbM, Kabat, and Contact numbering and the mature VH, VL, heavy chain, and light chain.

### Example 2: Binding of Anti-Human CD73 Monoclonal Antibodies to Cell Surface CD73

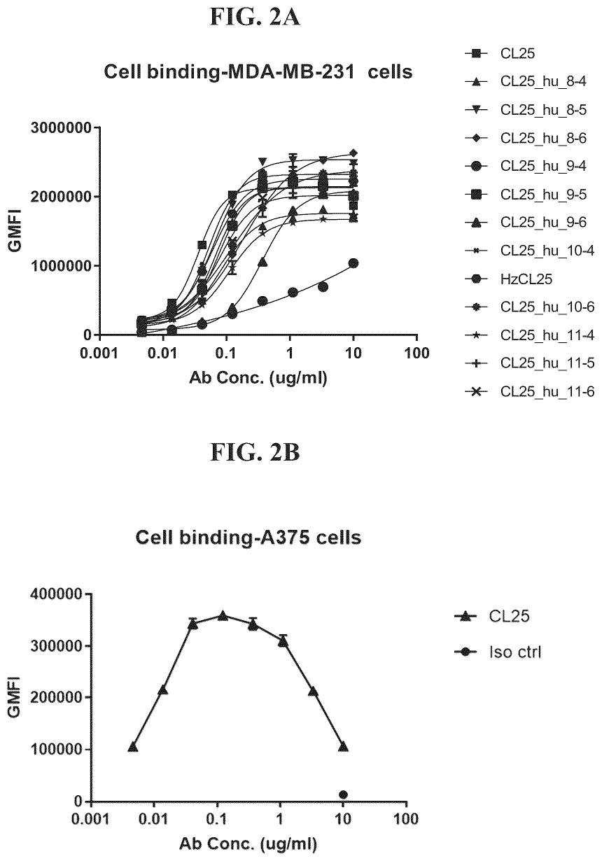

To test the binding of humanized and non-humanized CL25 clones to cell surface CD73, MDA-MB-231 or A375 cells were washed and added to 96-well plates at 5×10⁴ cells/well. The cells were stained with the indicated concentration of antibodies for 30 minutes on ice (FIG. 2A and FIG. 2B). Next, the cells were washed and stained using goat anti-mouse secondary conjugated to phycoerythrin (PE) for 30 minutes on ice. The cells were then washed and analyzed by flow cytometry. Geometric mean fluorescence intensity (GMFI) of CD73 staining was graphed (FIG. 2A and FIG. 2B). Both CL25 and HzCL25 displayed high potency binding to cells with high levels of surface CD73 (MDA-MB-231 cells) and moderate levels of surface CD73 (as tested in A375 cells).

### Example 3: Anti-Human CD73 Monoclonal Antibody-Mediated Cellular CD73 Inhibition

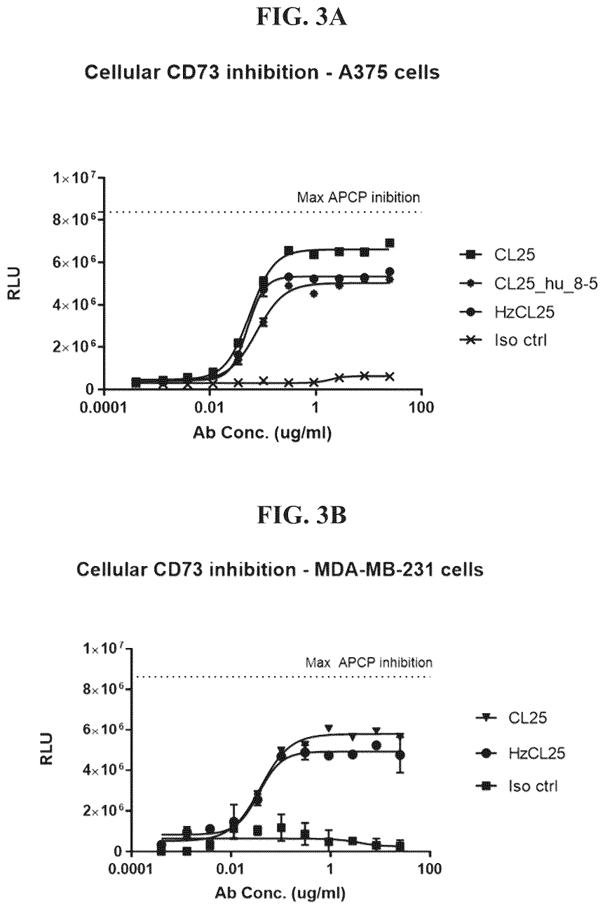

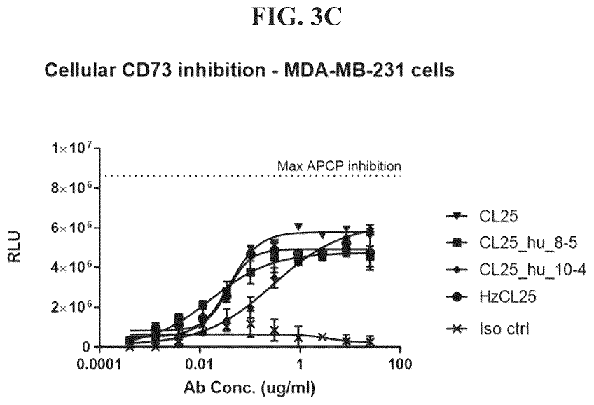

To measure ability of anti-CD73 antibody to inhibit CD73 activity on cells, A375 and MDA-MB-231 cells were washed with serum free RPMI media (ThermoFisher) and plated in 96-well plates at a concentration of 8×10⁴ cells/well for A375 or 1×10⁴ cells/well for MDA-MB-231. The cells were incubated with the indicated concentration of antibodies or APCP at 37° C. 5% CO₂ for 30 minutes (FIG. 3A, FIG. 3B, and FIG. 3C). Next, adenosine monophosphate (AMP) was added to a final concentration of 100 μM and cells were incubated an additional 3 hours at 37° C. 5% CO₂. Plates were centrifuged for 1-2 minutes at 300 g and 25 μL of supernatant was transferred into a new 96-well plates. AMP-Glo Assay was used according to the manufacturer's instructions (Promega). Relative luminescence unit (RLU) is directly correlated with the AMP concentration in this assay. Results are depicted in FIG. 3A, FIG. 3B, and FIG. 3C.

Both CL25 and HzCL25 had good potency in inhibiting cellular CD73 in both tested cell types (FIG. 3A, FIG. 3B, and FIG. 3C). HzCL25 had a similar ability as CL25 to inhibit cellular CD73 (FIG. 3A, FIG. 3B, and FIG. 3C).

### Example 4: Anti-Human CD73 Monoclonal Antibody-Mediated Soluble CD73 Inhibition

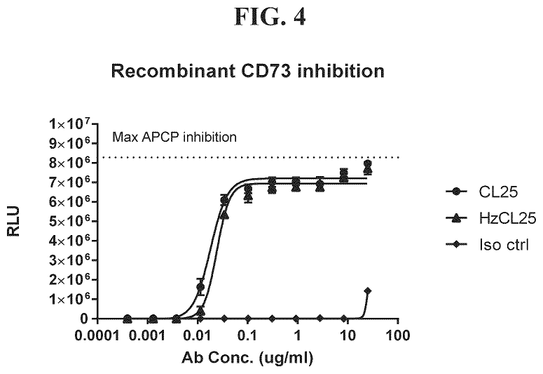

To measure the ability of the CD73 antibodies to inhibit CD73 activity of recombinant protein, recombinant human CD73 (rhuCD73) (SEQ ID NO:70) was added to 96-well plates at a final concentration of 0.008 μg/mL with the indicated concentration of antibodies (FIG. 4) or adenosine 5′-[α,β-methylene]diphosphate (APCP) and incubated at 37° C. 5% CO₂ for 30 minutes. After the 30 minute incubation, AMP was added to a final concentration of 100 μM and the reactions were incubated an additional 3 hours at 37° C. 5% CO₂. 25 μL of supernatant was transferred into new 96-well plates. The AMP-Glo Assay was used according to the manufacturer's instructions. RLU is a directly correlated with the AMP concentration in this assay. Results are depicted in FIG. 4. Both CL25 and HzCL25 showed high potency and no hook-effect (FIG. 4). HzCL25 had a similar ability as CL25 to inhibit cellular CD73 (FIG. 4).

### Example 5: Anti-Human CD73 Monoclonal Antibody-Mediated Reversal of AMP-Mediated Suppression of T Cell Proliferation

To measure the ability of the CD73 antibodies to reverse AMP-mediated suppression of T cell proliferation, primary human CD4+ T cells were purified from peripheral blood mononuclear cells (PBMCs) using a human CD4+ T-cell isolation kit (Miltenyi Biotec). Isolated CD4⁺ T cells were labeled with 1 μM of carboxyfluorescein succinimidyl ester (CFSE) (BD Biosciences) according to the manufacturer's protocol. CFSE labeled cells were resuspended in RPMI containing 10% fetal bovine serum and 60 IU/ml recombinant human IL-2. Approximately 50,000 cells/well were added in round bottom 96-well plate. Dynabeads human T activator CD3/CD28 beads were added to cell suspension at bead:cell ratio 1:1 and incubated for 1 hour at 37 degrees C. Serial dilutions of antibodies were added into the designated wells and incubated for 30 minutes at 37 degrees C. Finally AMP was added at a final concentration of 1000 μM and the whole culture was incubated for 5 days at 37 degrees C. in the incubator. After 5 days, CD4+ T cell proliferation was determined by CFSE based flow cytometry analysis using LSRFORTESSA X-20 analyzer (BD Biosciences).

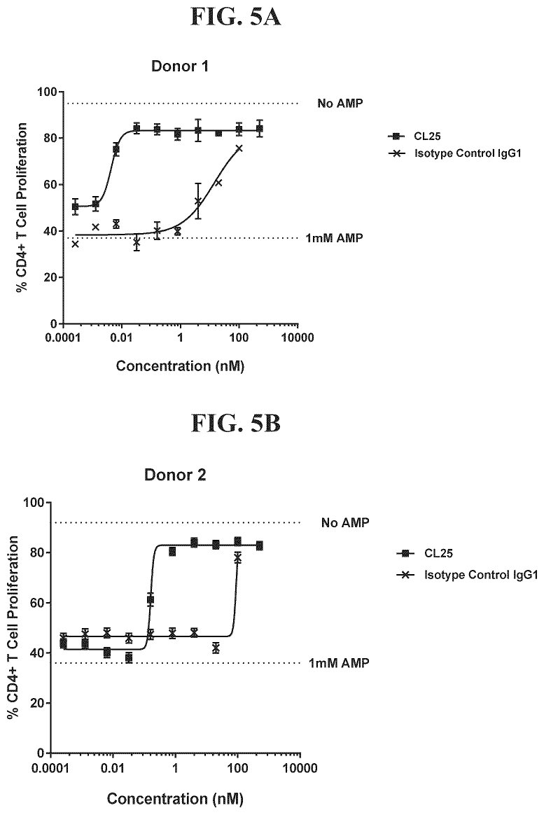

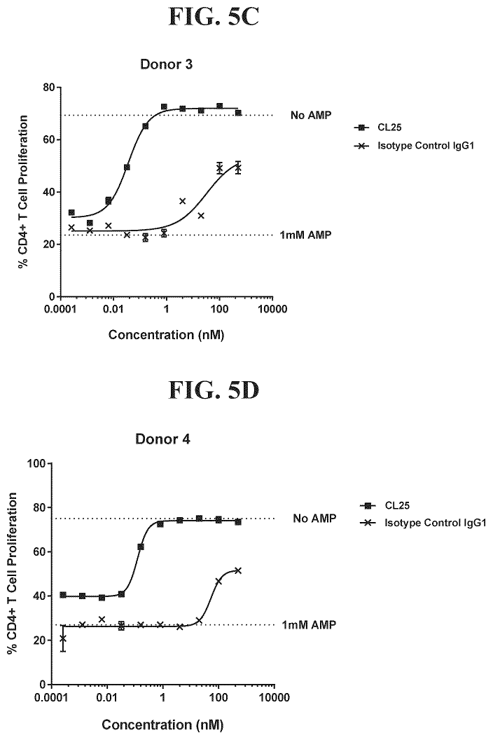

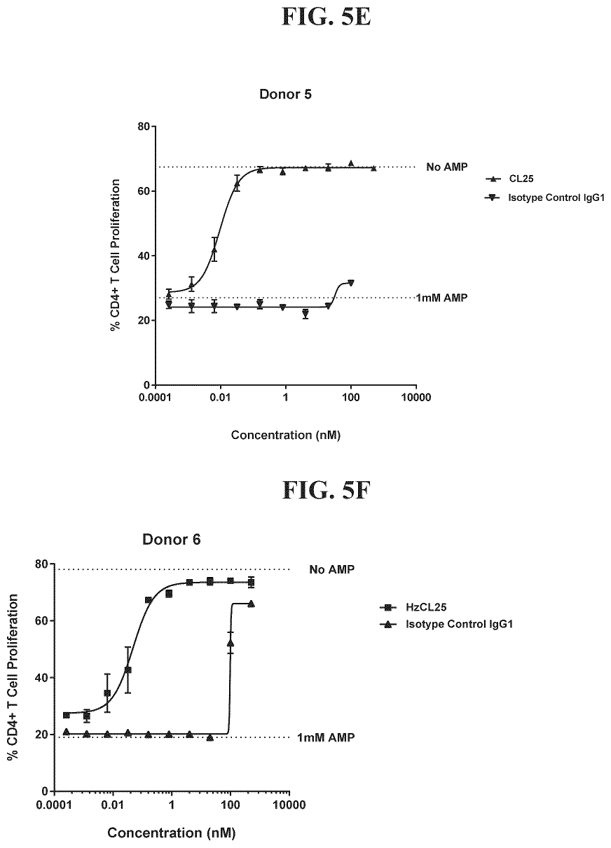

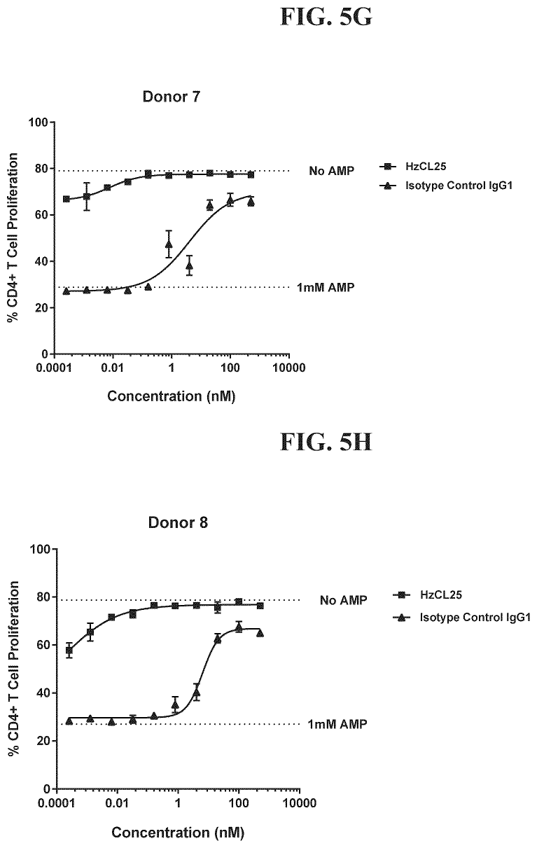

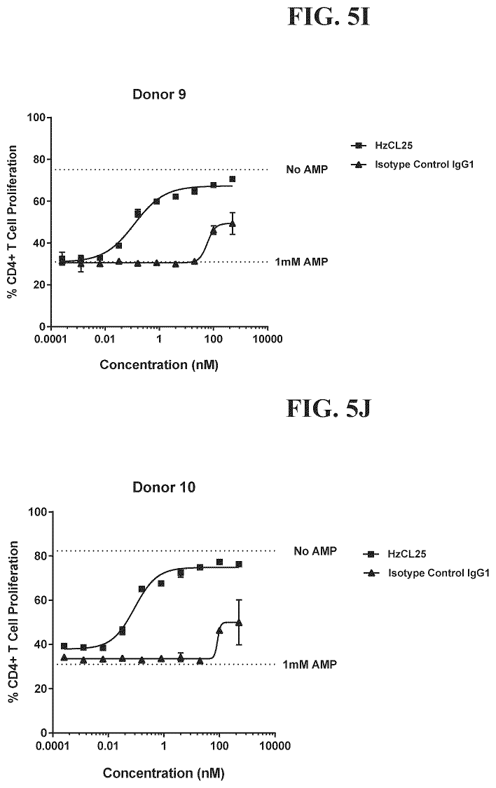

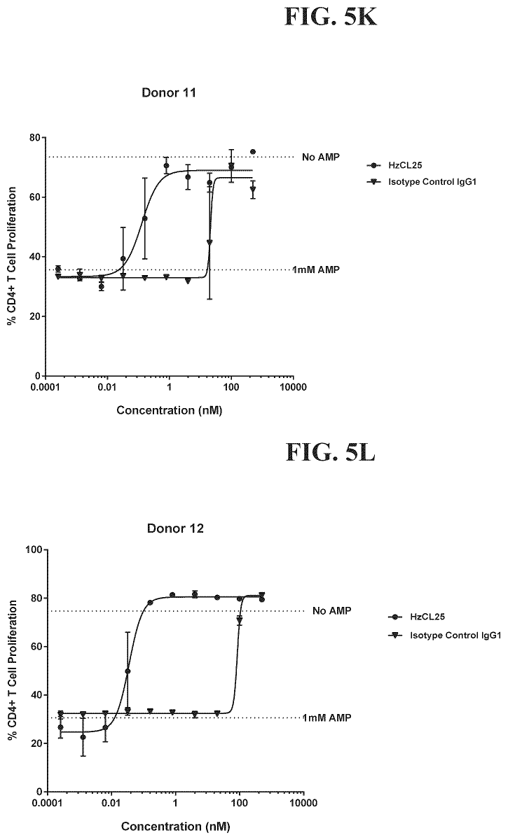

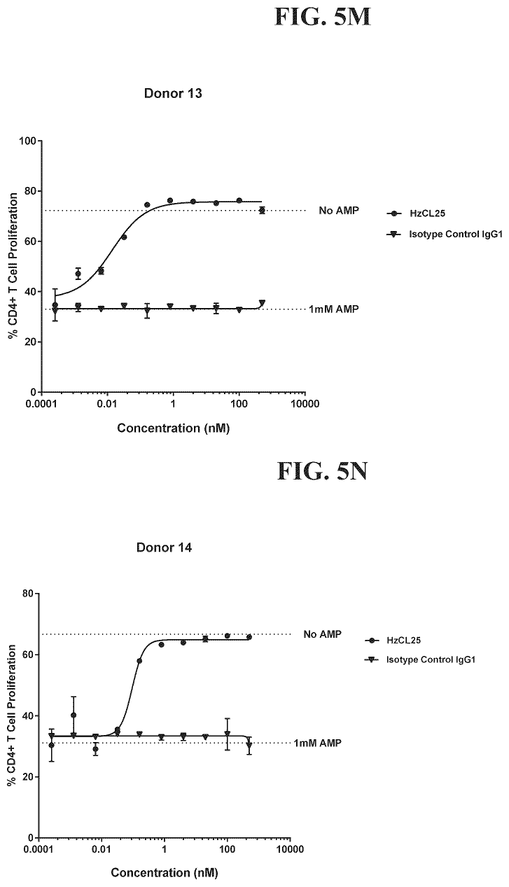

Anti-CD73 antibody reversed AMP-mediated suppression of CD4+ T cell proliferation in a concentration dependent manner in multiple different human donors (FIGS. 5A-5N).

### Example 6: Binding Affinity

CD73 enzymatic activity requires substrate binding in the open conformation. After substrate binding, CD73 has to go through a large conformational change from open to closed conformation to convert AMP to adenosine. Antibody binding that inhibits or modulates this conformational change will potentially decrease the rate of AMP to adenosine conversion.

To assess the binding affinity of HzCL25, surface plasmon resonance (SPR) was performed using a Biacore 8K instrument (GE Healthcare) at 25° C. The SPR running buffer (10 mM HEPES, 150 mM NaCl, 3 mM EDTA and 0.05% v/v Surfactant P20, pH 7.4) was prepared from 10×HBS-EP Buffer (GE Healthcare). Anti-human Fc antibodies (GE Healthcare) were immobilized via amine coupling on all sixteen flow cells of an S series sensor chip CM5 (GE Healthcare). The immobilization levels were ˜9000 RU for all flow cells. The desired capturing level of anti-CD73 antibody was achieved by flowing appropriate concentration of anti-CD73 antibody through the active flow cell of each channel. The non-cleavable ADP analogue APCP (adenosine-5′-(α,β-methylene)diphosphate) with the presence of Zn²⁺ can be used to shift the CD73 conformational equilibrium from open towards closed. Therefore, recombinant CD73 was incubated with SPR running buffer in the presence of 100 μM APCP and 10 μM ZnCl₂ (closed SPR running buffer) to study the binding of anti-CD73 antibody to the CD73 in the closed conformation. To achieve this, the ABA injection feature in Biacore 8K was used. For open conformation, the ABA injection sequence started with 60 seconds injection of running buffer. Then, CD73 3-fold serial dilution concentration series prepared from CD73 stock (BPS Bioscience) and running buffer were injected for 180 seconds immediately followed by running buffer for 240 seconds. For closed conformation, the normal SPR running buffer was replaced by closed SPR running buffer. Surface was regenerated with 30 seconds injection of 3 M MgCl₂. Binding kinetics and affinity parameters were obtained from a global fit of the data to 1 to 1 binding model. Binding affinities and kinetic association and dissociation rate constants to human, cynomolgus, and mouse CD73 in either open or closed conformations are shown in Table 6 below. The results in Table 6 ensure cynomolgus pharmacokinetic data will reflect human pharmacokinetic data.

TABLE 6
Binding affinities and kinetic association and dissociation rate constants to human
(SEQ ID NO: 70), cynomolgus (SEQ ID NO: 72), and mouse (SEQ ID NO: 71)
CD73 in either open or closed conformations for the indicated antibodies (Ab).

| | | Open | | | Closed | | |
|---|---|---|---|---|---|---|---|
| Ab | CD73 | kₐ (1/Ms) | k_{d} (1/s) | K_{D} (M) | kₐ (1/Ms) | k_{d} (1/s) | K_{D} (M) |
| CL25 | human | >1E+06 | 3.94E−04 | <3.94E−10 | 9.03E+05 | 3.44E−04 | 3.81E−10 |
| | Cyno- | >1E+06 | 6.58E−04 | <6.58E−10 | 9.36E+05 | 4.95E−04 | 5.29E−10 |

| | molgus | | | | |
|---|---|---|---|---|---|
| | Murine | No Binding | | | No Binding |
| HzCL25 | Human | >1E+06 | 4.49E−04 | <4.49E−10 | Not performed |
| | Cyno- | >1E+06 | 4.68E−04 | <4.68E−10 | Not performed |
| | molgus | | | | |
| | Murine | No binding | | | Not performed |
| CL25_ | Human | 7.70E+05 | 1.76E−03 | 2.29E−09 | Not performed |
| hu_8-4 | | | | | |
| CL25_ | Human | >1E+06 | 9.49E−04 | 7.18E−10 | Not performed |
| hu_8-5 | | | | | |
| CL25_ | Human | 7.15E+05 | 1.11E−03 | 1.55E−09 | Not performed |
| hu_8-6 | | | | | |
| CL25_ | Human | 3.82E+05 | 2.64E−03 | 6.90E−09 | Not performed |
| hu_9-4 | | | | | |
| CL25_ | Human | 5.65E+05 | 1.14E−03 | 2.02E−09 | Not performed |
| hu_9-5 | | | | | |
| CL25_ | Human | 4.24E+05 | 9.53E−04 | 2.25E−09 | Not performed |
| hu_9-6 | | | | | |
| CL25_ | Human | 8.87E+05 | 1.07E−03 | 1.20E−09 | Not performed |
| hu_10-4 | | | | | |
| CL25_ | Human | 5.24E+05 | 9.20E−04 | 1.75E−09 | Not performed |
| hu_10-6 | | | | | |
| CL25_ | Human | 6.24E+05 | 1.55E−03 | 2.48E−09 | Not performed |
| hu_11-4 | | | | | |
| CL25_ | Human | 7.99E+05 | 1.17E−03 | 1.46E−09 | Not performed |
| hu_11-5 | | | | | |
| CL25_ | Human | 5.50E+05 | 1.01E−03 | 1.84E−09 | Not performed |
| hu_11-6 | | | | | |

### Example 7: Epitope Mapping

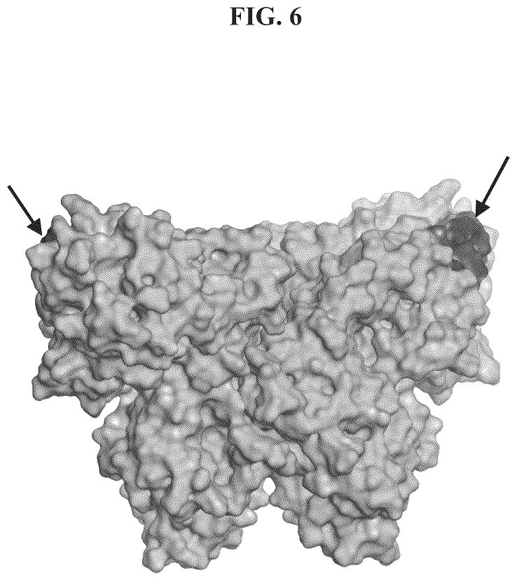

To map the epitope of CL25, Hydrogen-deuterium exchange mass spectrometry (HDX) was performed. CD73 was incubated in deuterium oxide either alone or in complex with CL25 Fab. The deuterium exchange was carried out at 20° C. for 0 seconds, 60 seconds, 600 seconds, or 3600 seconds. The exchange reaction was quenched by low pH and the proteins were digested with pepsin/protease VIII. The deuterium levels at the identified peptides were monitored from the mass shift on LC-MS. The deuterium buildup curves over exchange time for all the peptides were plotted vs time. Peptides with significant reduction in deuterium uptakes upon binding to Fab were assigned as the epitopes for each antibody. The epitope determined by HDX-MS for CL25 is mapped onto the crystal structure of human CD73 (4H2F.pdb) (FIG. 6) and is TKVQQIRRAEPNVL (SEQ ID NO:76) (i.e., amino acids 40-53 of SEQ ID NO:70).

### Example 8: CD73 Cell Surface Levels

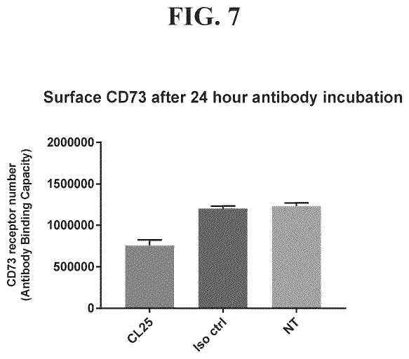

To measure the amount of CD73 on the cell surface after antibody treatment, MDA-MB-231 cells were re-suspended in media (10% FBS RPMI-1640) and plated in 96-well plates at 1×10⁵ cells/well. Indicated antibodies were added at a final concentration of 10 μg/mL and plates were incubated at 37° C. 5% CO₂ for 24 hours. Cells were recovered using Versene and transferred to new 96-well plates. Cells were washed and stained for 30 minutes on ice with 10 μg/mL of non-competing antibody directly conjugated to Dy650. Cells were washed and analyzed by flow cytometry. CD73 cell surface receptor density was determined by Antibody Binding Capacity (ABC) using Quantum Simply Cellular beads. Treatment of cells with CL25 for 24 hours decreased the levels of cell surface CD73 (FIG. 7).

### Example 9: Effect of Anti-CD73 Antibody on Tumor Growth

The in vivo efficacy of the CL25 antibody was tested. HzCL25 was suspended in 1× phosphate buffered saline (PBS) (Life Technologies) for intraperitoneal dosing of hu-CD34 NSG mice (Jackson laboratories). 1×PBS and Fc disabled human IgG1 suspended in 1×PBS was included in this study as a control. Mice with humanized immune systems were purchased from Jackson Labs (Bar Harbor, Maine). Briefly, 3 week old female NSG/NOD SCID mice received a single dose of irradiation toxic to immune cell precursors and were then “rescued” by injection of human cord blood, CD34⁺ selected cells. After 12 weeks (to allow recovery and engraftment of the human immune cells), the mice were received at the AALAS certified vivarium at the Incyte Research Institute. The mice comprised recipients of three distinct human immune donors to better represent individual variations in immune response.

The left flank of the mice were shaved the day prior to inoculation with 5×10⁶ cells of the human melanoma line A375 (ATCC, Manassas Virginia) suspended in matrigel (Corning Life Sciences, Tewksbury, Mass). On day 11, tumor dimensions were measured by Vernier calipers, and volume estimated by the formula Volume=[L (long dimension)×W2 (short dimension)]/2. Mice were randomized into 3 groups of 5 or 6 mice of approximate mean volume (˜200 mm³) and donor representation. Tumors were measured every 2-4 days for the duration of the study.

HzCL25 was diluted in PBS to concentrations of 1 and 0.1 mg/ml, and a matching isotype control (cx00376-001), was diluted to 1 mg/ml fresh on the morning of administration. Every six days, from day 11, antibody was given to the mice by intraperitoneal injection at 10 ml/kg, achieving effective doses of 1 and 10 mg/kg of body weight. A total of three doses were given.

Forty-eight hours after the third and final dose, mice were euthanized by CO₂ asphyxiation. Tumors were excised, placed into media containing proteases, placed on ice, and then disrupted into single cell suspensions by use of the GentleMacs Tissue Disruptor (Miltenyi, Auburn CA). The cells remained on ice while being processed for flow cytometry and enzymatic assay.

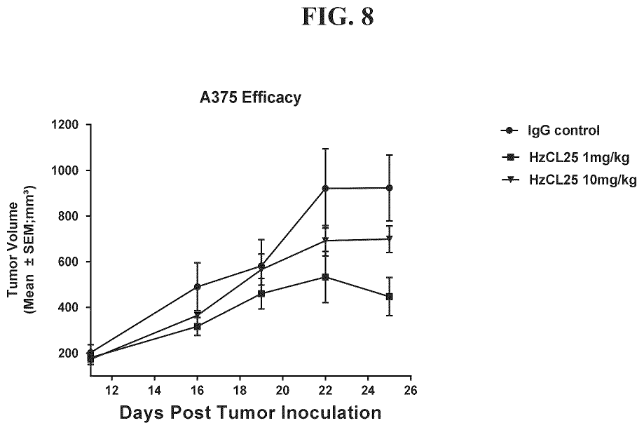

Mice administered HzCL25 exhibited tumor growth inhibition at both doses; the low dose significantly (p=0.026, two tailed t test) slowed growth 56% (FIG. 8).

### Example 10: Ex Vivo Enzymatic Activity Assay

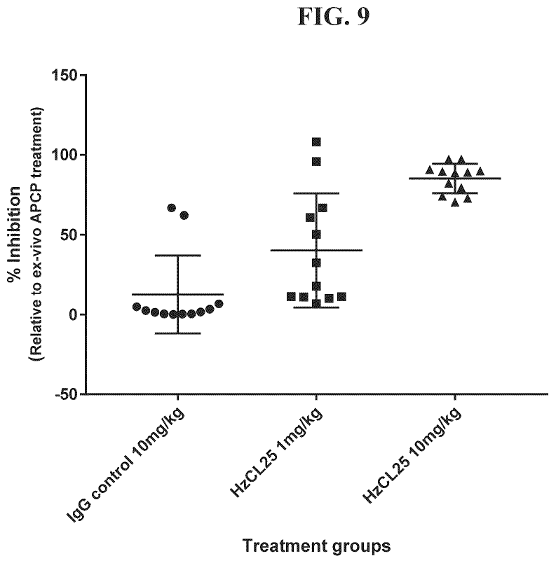

To evaluate the enzymatic activity of CD73 in tumor-bearing mice administered HzCL25, single cell suspension of the tumor homogenates from the experiment of FIG. 8 were sorted for live cells using Dead Cell Removal MicroBeads (Miltenyi Biotec). Post cell selection, 10,000 live cells per well were plated with 100 μM of AMP. As a positive control, 250 μM APCP treated cells were used. Following treatment, AMP-glo kit (Promega; Cat. #V5011) was used to detect enzymatic activity of CD73 as per the manufacturer's guidelines. Luminescence was used as a measure for AMP level detection and readouts from each animal were normalized to APCP-treated culture readouts, where APCP-treated cultures were considered to demonstrate maximum inhibition of CD73 activity (FIG. 9). As shown in FIG. 9, HzCL25 demonstrated a dose-dependent inhibition of CD73 enzymatic activity upon tumor cells.

### Example 11: Free Surface CD73 of Anti-CD73-Treated Tumors

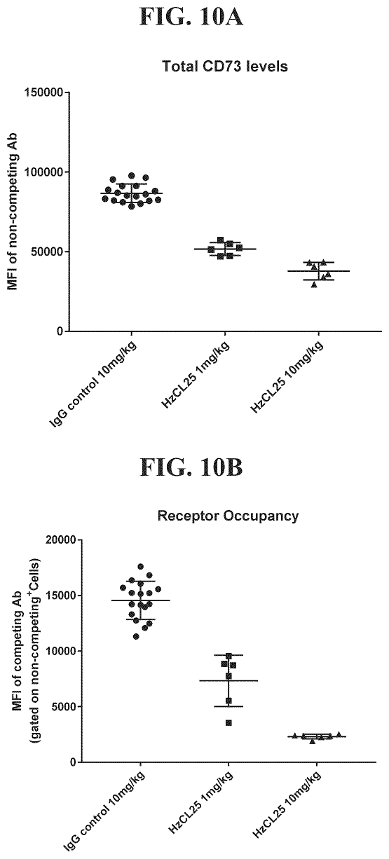

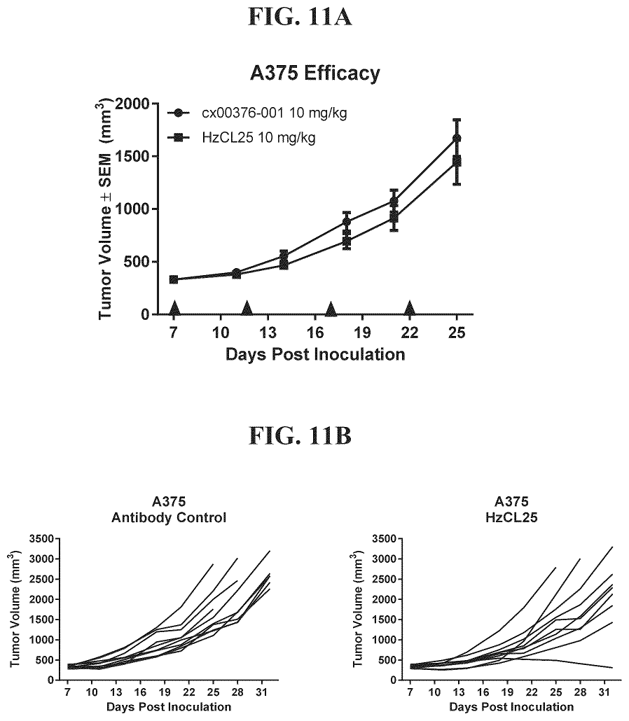

To evaluate the receptor coverage post-treatment, single cell suspensions of tumor homogenates from the experiment of FIG. 8 were stained with fluorochrome-conjugated antibodies against viability dye (Biolegend, Cat. #423110), human CD45 (Biolegend, Cat. #304036), mouse CD45 (BD Biosciences, Cat. #563410), mouse CD90.2 (BD Biosciences, Cat. #565257), and competing (AF488-conjugated CL25 antibody) or non-competing antibody (PE-conjugated Antibody X). Cells were run and analyzed using BD FACSymphony flow cytometry analyzer and FlowJo software package respectively. Analysis was performed by gating on live cell population, followed by mouse cells and human immune cell exclusion, focusing on the tumor cell population. Total CD73 levels on tumor cells were detected using fluorophore tagged non-competing CD73 antibody. In a dose-dependent manner, HzCL25 decreased the total CD73 levels on tumor cells (FIG. 10A). Of the cells expressing CD73 as measured in FIG. 10A, free surface CD73 was measured using a fluorophore tagged competing (CL25) antibody (FIG. 10B). HzCL25 treatment demonstrated a dose-dependent decrease in free surface CD73 (FIG. 10B). Furthermore, this reduction in free surface CD73 levels for both dosages was significantly lower compared to IgG treated tumors (FIG. 10B).

### Example 12: Pharmacokinetic and Pharmacodynamics In Vivo Studies of Anti-CD73 Antibody

To assess the efficacy of HzCL25 in vivo, female human CD34⁺ reconstituted mice (21 weeks of age; The Jackson Laboratory, Bar Harbor, ME) were inoculated subcutaneously with 5×10⁶ A375 human melanoma cells (ATCC #CRL-1619). The treatment of tumor-bearing mice was started 7 days post-inoculation, when tumor volume reached approximately 330 mm³. A375 engrafted humanized NSG mice were dosed intraperitoneally with HzCL25 at 10 mg/kg every 5 days. The study animals received 6 total doses of antibody, with the final dose on day 32. Tumors were collected from mice with tumors greater than 10% of their body weight on days 26 and 29, and from all remaining animals on day 33. Serum was collected from all animals on day 26, again from mice with tumors greater than 10% of their body weight on day 29, and from all remaining animals on day 33. Through 26 days (at which time the control IgG group had reached its mean efficacy endpoint), HzCL25 provided modest but insignificant tumor growth inhibition of 14% (FIG. 11A). However, extended dosing through 32 days showed marked differences in tumor growth rates and survival between HzCL25-treated animals and those remaining mice receiving the IgG control (FIG. 11B).

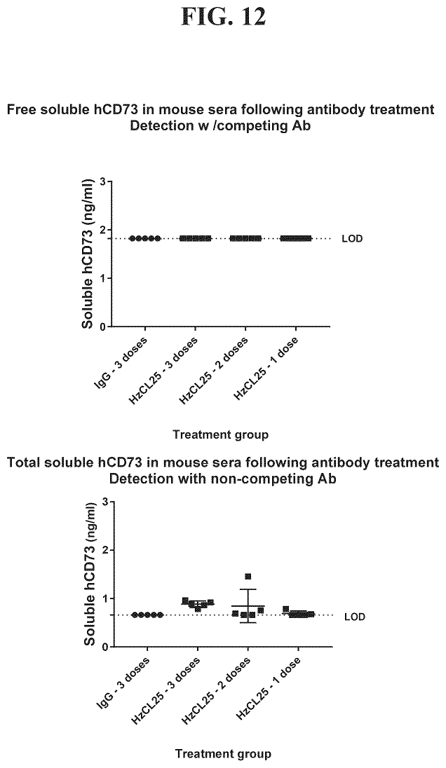

A separate pharmacodynamic (PD) study was initiated on day 22 with HzCL25 at 10 mg/kg every 3 days for a total of 2 doses of antibody. Tumors from PD animals were collected on day 27, 48 hours post-second antibody dose. To measure soluble human CD73 (hCD73) in mouse sera following antibody treatment, MSD plates were coated with a non-competing capture IgG antibody at 1 μg/mL and incubated at 4° C. overnight. Plates were washed and 1:5 diluted mouse serum samples were added. A standard curve was generated using recombinant human CD73 (rhuCD73). Plates were incubated for 2 hours at room temperature with shaking at 600 rpm. Plates were washed and competing (HzCL25) or non-competing (Antibody X) antibodies directly conjugated to sulfo-tag were added to the plate at 1 μg/mL and incubated for 1 hour at room temperature with shaking at 600 rpm. Plates were washed and 150 μL of Read Buffer were added and analyzed using an MSD plate reader. Soluble hCD73 levels from mouse sera were calculated from the standard curve. Although treatment with HzCL25 slightly stabilized soluble human CD73 in mice, the level of soluble human CD73 was still far below the level of antibody in the mice (FIG. 12).

### Example 13: Generation of Anti-Human CD73 Monoclonal Antibody 3-F03

To generate additional anti-human CD73 monoclonal antibodies, multiple selection rounds of a single donor library were performed. The library of approximately 1.5E12 phage particles was enriched over three rounds of panning using 200 nM biotinylated human CD73 (SEQ ID NO:70). The scFv cassettes from this pool were then recombined into a yeast display vector and a library of approximately 5.4E7 was created. This library was selected by FACS for three rounds using 100 nM biotinylated murine CD73 (SEQ ID NO:71). Unique sequences were obtained from the final sorting output by Sanger sequencing of yeast colonies. The yeast 3-F03 scFv sequence was identified from this pool and contained a VH of the amino acid sequence set forth in SEQ ID NO:77 and a VL of the amino acid sequence set forth in SEQ ID NO:65.

To construct a full-length human 3-F03 antibody, the yeast 3-F03 scFv sequences were modified prior to cloning into a human IgG1 scaffold comprising the human IgG1 constant region set forth in SEQ ID NO:73 and the human kappa light chain constant region set forth in SEQ ID NO:74. For the VH, the N-terminal glutamate (E) of SEQ ID NO:77 was removed and the threonine (T) at Kabat position H77 of SEQ ID NO:77 (i.e., position 78 of SEQ ID NO:77) was substituted with an alanine (A). For the VL, the N-terminal alanine (A) of SEQ ID NO:65 was removed. The resulting full-length human 3-F03 antibody contains the VH and VL set forth in the amino acid sequences of SEQ ID NOs:60 and 61, respectively. The resulting full-length human 3-F03 antibody contains the heavy chain and light chain set forth in the amino acid sequences of SEQ ID NOs: 66 and 31, respectively. This antibody is referred to herein as “3-F03”. Table 3, above, shows the amino acid sequences of the 3-F03 CDRs according to IMGT, Chothia, AbM, Kabat, and Contact numbering and of the mature VH, VL, heavy chain, and light chain.

### Example 14: Binding of 3-F03 to Cell Surface CD73

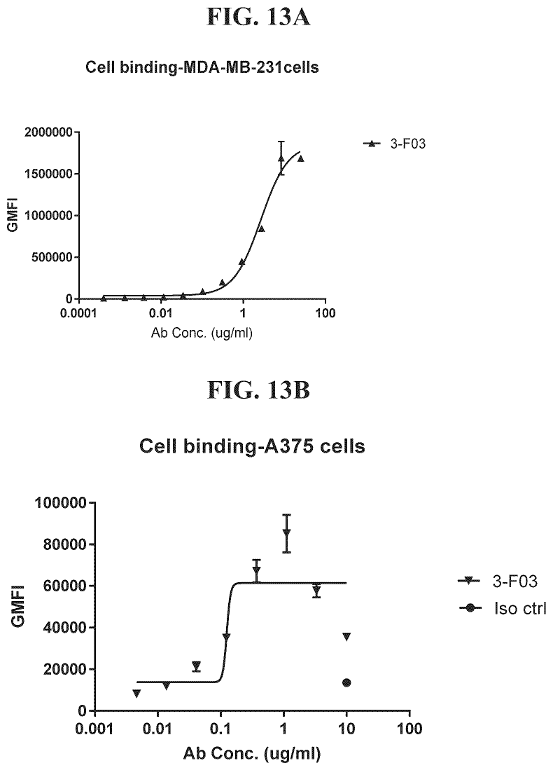

The binding of 3-F03 to cell surface CD73 was performed as described in Example 2, above. 3-F03 displays high potency binding to cells with high levels of surface CD73 (MDA-MB-231), and moderate levels of CD73 (A375 cells) (FIG. 13A and FIG. 13B).

### Example 15: 3-F03-Mediated Cellular CD73 Inhibition

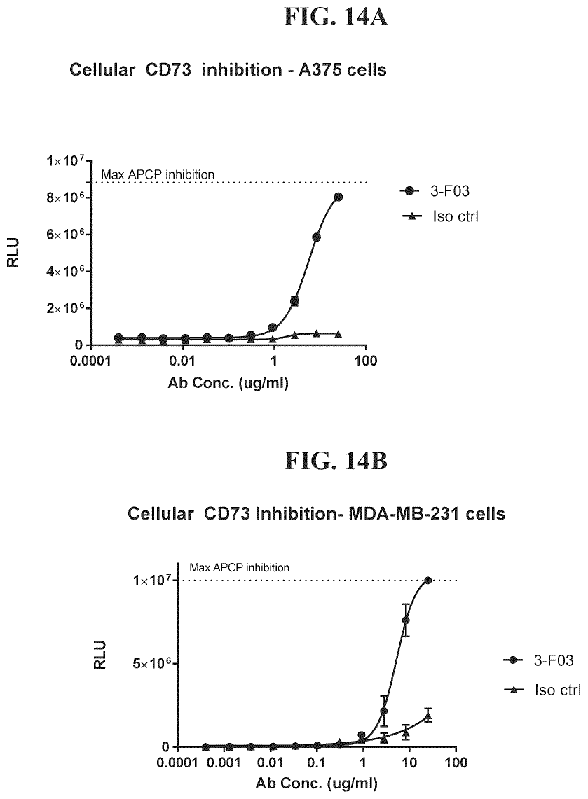

The ability of 3-F03 to inhibit CD73 activity on cells was evaluated as described in Example 3, above. Results are depicted in FIG. 14A and FIG. 14B.

Clone 3-F03 showed maximum inhibition of cellular CD73 in both tested cell types as compared to the small molecule inhibitor of CD73, APCP (FIG. 14A and FIG. 14B).

### Example 16: 3-F03-Mediated Soluble CD73 Inhibition

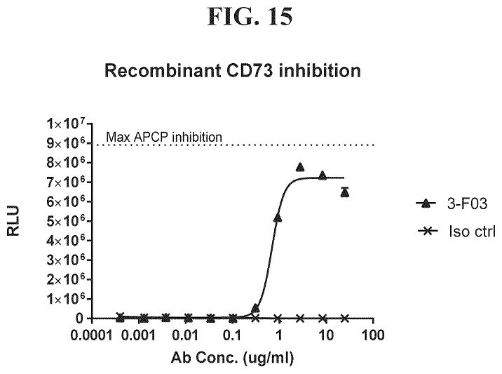

The ability of 3-F03 to inhibit CD73 activity of recombinant protein was evaluated as described in Example 4, above, except that 0.025 ug/mL of rhuCD73 was used. Results are depicted in FIG. 15. Antibody 3-F03 had good potency (FIG. 15). Antibody 3-F03 did not exhibit any hook-effect.

### Example 17: Anti-Human CD73 Monoclonal Antibody-Mediated Reversal of AMP-Mediated Suppression of T Cell Proliferation

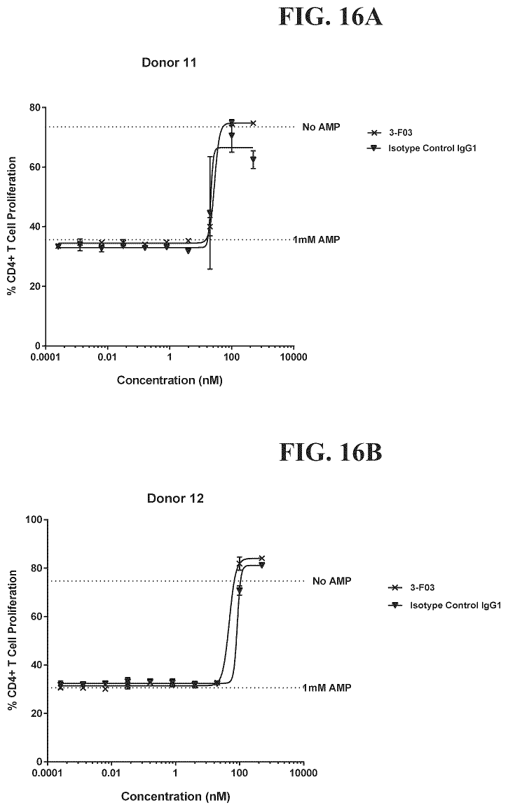

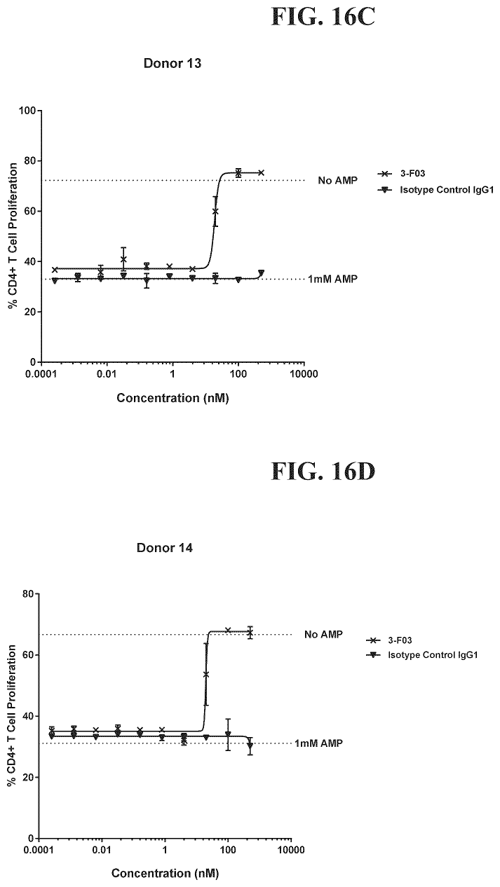

The ability of 3-F03 to reverse AMP-mediated suppression of T cell proliferation was evaluated as described in Example 5, above. 3-F03 reversed AMP-mediated suppression of CD4+ T cell proliferation in a concentration dependent manner in multiple different human donors (FIGS. 16A-16D).

### Example 18: Binding Affinity of Anti-CD73 Antibody

The binding affinity of 3-F03 was evaluated as described in Example 6, above. Binding affinities and kinetic association and dissociation rate constants to human, cynomolgus, and mouse CD73 in either open or closed conformations are shown in Table 7 below.

TABLE 7
Binding affinities and kinetic association and dissociation rate constants to
human, cynomolgus, and mouse CD73 in either open or closed conformations.

| | | Open | | | Closed | | |
|---|---|---|---|---|---|---|---|
| Sample | | kₐ | k_{d} | K_{D} | kₐ | k_{d} | K_{D} |
| Name | CD73 | (1/Ms) | (1/s) | (M) | (1/Ms) | (1/s) | (M) |

| | | | | | |
|---|---|---|---|---|---|
| 3-F03 | human | 2.15E+05 | 7.96E−05 | 3.70E−10 | No Binding |
| | Cynomolgus | 3.01E+05 | 2.21E−04 | 7.34E−10 | No Binding |
| | Murine | 2.17E+05 | 3.60E−04 | 1.66E−09 | No Binding |

### Example 19: Epitope Mapping of 3-F03

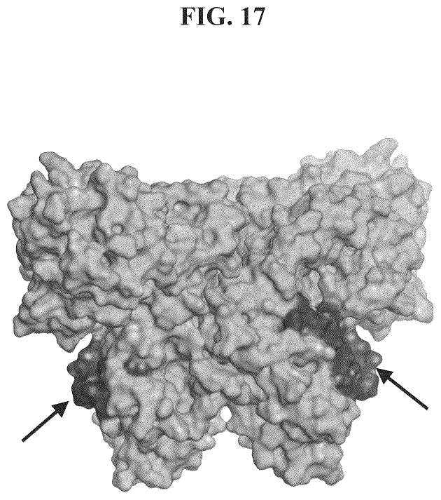

The epitope of 3-F03 was mapped as described in Example 7, above. The epitopes determined by HDX-MS for 3-F03 are mapped onto the crystal structure of human CD73 (4H2F.pdb) (FIG. 17) and are AAVLPFGGTFDLVQ (SEQ ID NO:78) (i.e., amino acids 386-399 of SEQ ID NO:70) and ILPNFLANGGDGFQMIKDEL (SEQ ID NO:79) (i.e., amino acids 470-489 of SEQ ID NO:70).

### Example 20: Effect of 3-F03 on CD73 Cell Surface Levels

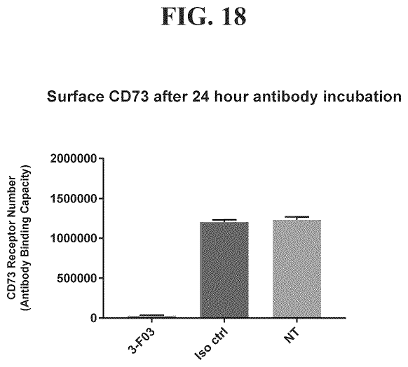

The amount of CD73 on the cell surface after treatment with 3-F03 was evaluated as described in Example 8, above. 3-F03 dramatically decreased the level of detectable CD73 on the cell surface compared to an isotype control antibody or non-treated cells (FIG. 18).

### Example 21: In Vivo Studies

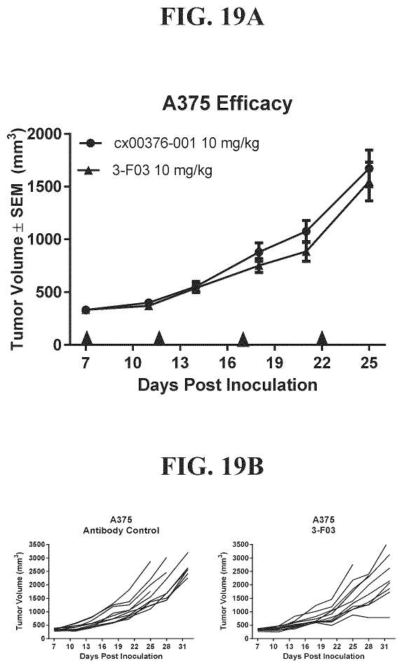

The in vivo efficacy of 3-F03 was evaluated as described in Example 12, above. Through 26 days, at which time, the control IgG group had reached its mean efficacy endpoint, 3-F03 provided modest but statistically insignificant tumor growth inhibition of 8% (FIG. 19A). However, extended dosing through 32 days showed marked differences in tumor growth rates and survival between 3-F03 treated animals and those remaining mice receiving the IgG control (FIG. 19B).

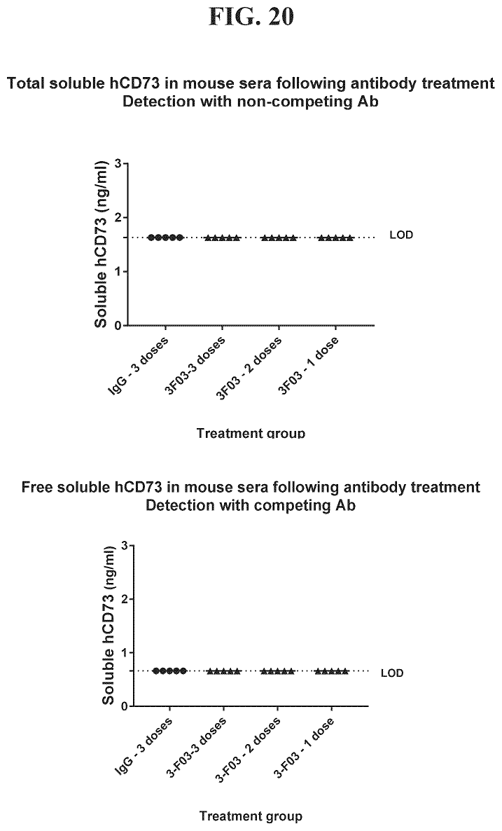

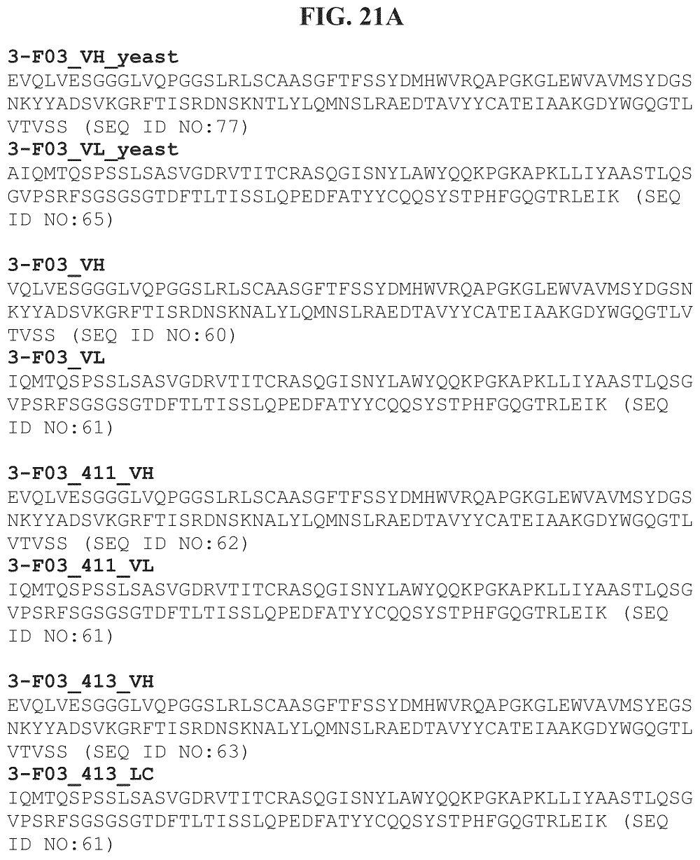

The PD properties of 3-F03 were also evaluated as described in Example 12, above. Treatment with 3-F03 did not stabilize soluble human CD73 in mice, as there was no detectable increase in total or free soluble human CD73 upon treatment with 3-F03 (FIG. 20).

### Example 22: 3-F03 Variants

Sequences of the 3-F03 light chain (LC, SEQ ID NO:66) and heavy chain (HC, SEQ ID NO:31) were used to construct a homology model. FIG. 21A-FIG. 21J provide the amino acid sequences of the VH and VL of exemplary 3-F03 variants. Antibodies comprising these VH and VL sequences contained the heavy chain constant region set forth in SEQ ID NO: 73 and light chain constant region set forth in SEQ ID NO:74. Table 8 provides the binding affinity and kinetics of the exemplary 3-F03 variants. None of the mutations tested dramatically impacted binding to CD73 by Biacore. All tested mutations had affinities within tenfold of the 3-F03 antibody, with the majority within two fold of 3-F03.

TABLE 8
Biacore binding affinity and kinetics of 3-F03 variants.

| | Mutation made (kabat numbering) | | | | | | |
|---|---|---|---|---|---|---|---|
| | | VH53 | | VL1 | | | |
| Sample | VH1 | (D, E, | VH77 | (A, D, | kₐ | k_{d} | K_{D} |
| Name | (E or —) | or S) | (A or T) | or —) | (1/Ms) | (1/s) | (M) |
| 3-F03_396 | E | D | T | A | 1.39E+05 | 1.78E−04 | 1.28E−09 |
| 3-F03_408 | E | D | T | — | 1.40E+05 | 1.86E−04 | 1.33E−09 |
| 3-F03_402 | — | D | T | A | 1.32E+05 | 1.84E−04 | 1.39E−09 |
| 3-F03_384 | E | D | T | D | 1.40E+05 | 1.98E−04 | 1.41E−09 |
| 3-F03_399 | E | D | A | A | 1.38E+05 | 1.97E−04 | 1.43E−09 |
| 3-F03_411 | E | D | A | — | 1.39E+05 | 2.04E−04 | 1.47E−09 |
| 3-F03_414 | — | D | T | — | 1.31E+05 | 1.98E−04 | 1.51E−09 |
| 3-F03_390 | — | D | T | D | 1.29E+05 | 2.12E−04 | 1.64E−09 |
| 3-F03_398 | E | E | T | A | 9.26E+04 | 1.59E−04 | 1.71E−09 |
| 3-F03_387 | E | D | A | D | 1.37E+05 | 2.38E−04 | 1.74E−09 |
| 3-F03_386 | E | E | T | D | 9.23E+04 | 1.64E−04 | 1.78E−09 |
| 3-F03_401 | E | E | A | A | 9.15E+04 | 1.67E−04 | 1.82E−09 |
| 3-F03_413 | E | E | A | — | 9.13E+04 | 1.71E−04 | 1.88E−09 |
| 3-F03_405 | — | D | A | A | 1.26E+05 | 2.39E−04 | 1.90E−09 |
| 3-F03_410 | E | E | T | — | 9.01E+04 | 1.76E−04 | 1.95E−09 |
| 3-F03_389 | E | E | A | D | 9.17E+04 | 1.89E−04 | 2.06E−09 |
| 3-F03_393 | — | D | A | D | 1.14E+05 | 2.39E−04 | 2.09E−09 |
| 3-F03_417 | — | D | A | — | 1.34E+05 | 2.84E−04 | 2.12E−09 |
| 3-F03_392 | — | E | T | D | 8.08E+04 | 1.80E−04 | 2.23E−09 |
| 3-F03_404 | — | E | T | A | 8.35E+04 | 1.89E−04 | 2.26E−09 |
| 3-F03_419 | — | E | A | — | 8.28E+04 | 2.00E−04 | 2.41E−09 |
| 3-F03_416 | — | E | T | — | 9.01E+04 | 2.21E−04 | 2.45E−09 |
| 3-F03_407 | — | E | A | A | 8.74E+04 | 2.35E−04 | 2.69E−09 |
| 3-F03_395 | — | E | A | D | 7.12E+04 | 2.10E−04 | 2.94E−09 |
| 3-F03_388 | E | S | A | D | 1.15E+05 | 8.68E−04 | 7.56E−09 |
| 3-F03_397 | E | S | T | A | 6.07E+04 | 4.89E−04 | 8.04E−09 |
| 3-F03_385 | E | S | T | D | 6.33E+04 | 5.38E−04 | 8.50E−09 |
| 3-F03_400 | E | S | A | A | 6.15E+04 | 5.29E−04 | 8.60E−09 |
| 3-F03_409 | E | S | T | — | 6.02E+04 | 5.46E−04 | 9.06E−09 |
| 3-F03_403 | — | S | T | A | 5.79E+04 | 5.41E−04 | 9.34E−09 |
| 3-F03_415 | — | S | T | — | 5.99E+04 | 6.06E−04 | 1.01E−08 |
| 3-F03_391 | — | S | T | D | 5.69E+04 | 5.84E−04 | 1.03E−08 |
| 3-F03_406 | — | S | A | A | 7.41E+04 | 7.65E−04 | 1.03E−08 |
| 3-F03_412 | E | S | A | — | 5.27E+04 | 6.38E−04 | 1.21E−08 |
| 3-F03_394 | — | S | A | D | 4.84E+04 | 6.30E−04 | 1.30E−08 |
| 3-F03_418 | — | S | A | — | 5.55E+04 | 7.99E−04 | 1.44E−08 |

— = absent.

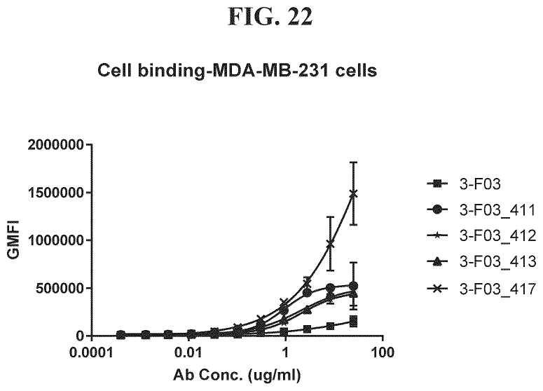

To test the binding of engineered 3-F03 variants to cell surface CD73, MDA-MB-231 cells were washed and added to 96-well plates at 5×10⁴ cells/well. Cells were stained with the indicated concentration of antibodies for 1 hour on ice. Cells were then washed and stained goat anti-mouse secondary conjugated to PE for 30 minute on ice. Cells were then washed and analyzed by flow cytometry. The GMFI of CD73 staining is graphed (FIG. 22). Each of the 3-F03 variants had a similar binding profile, except for 3-F03_417, which showed a slightly higher Ymax (FIG. 22). These data confirm the Biacore studies (Table 8): these mutations did not dramatically alter human CD73 binding for these variant clones.

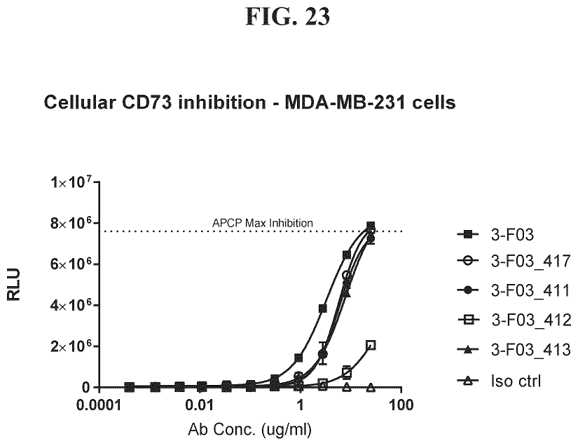

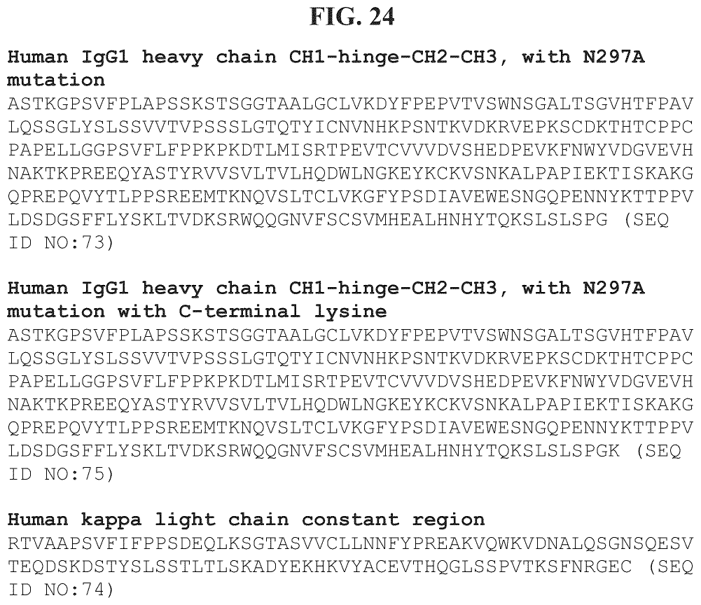

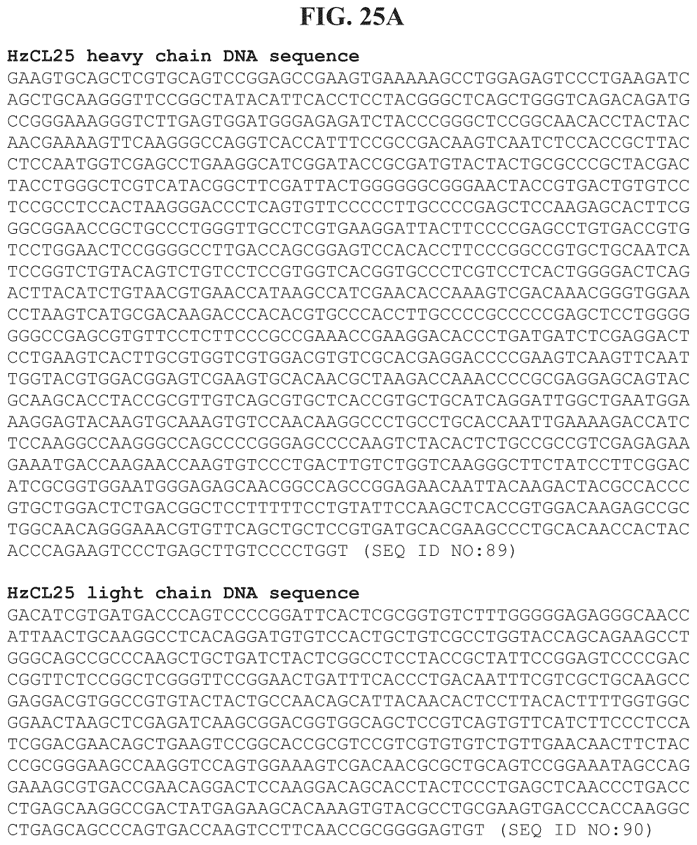

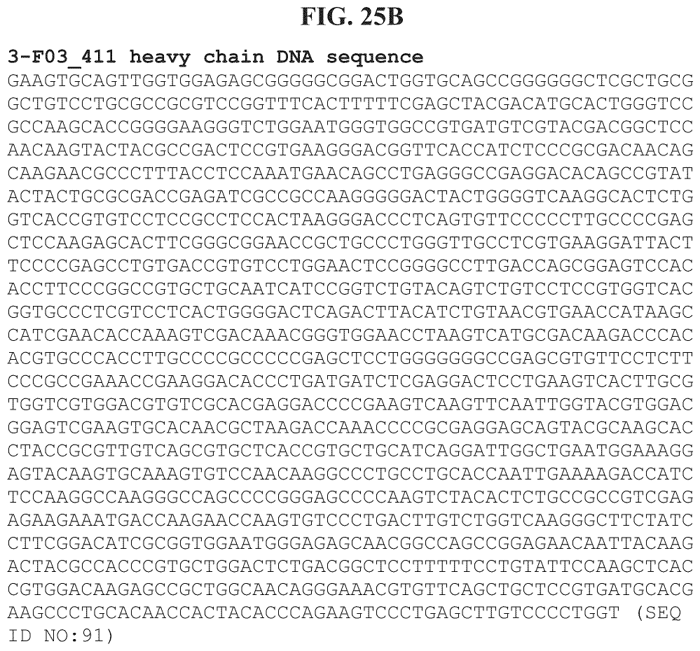

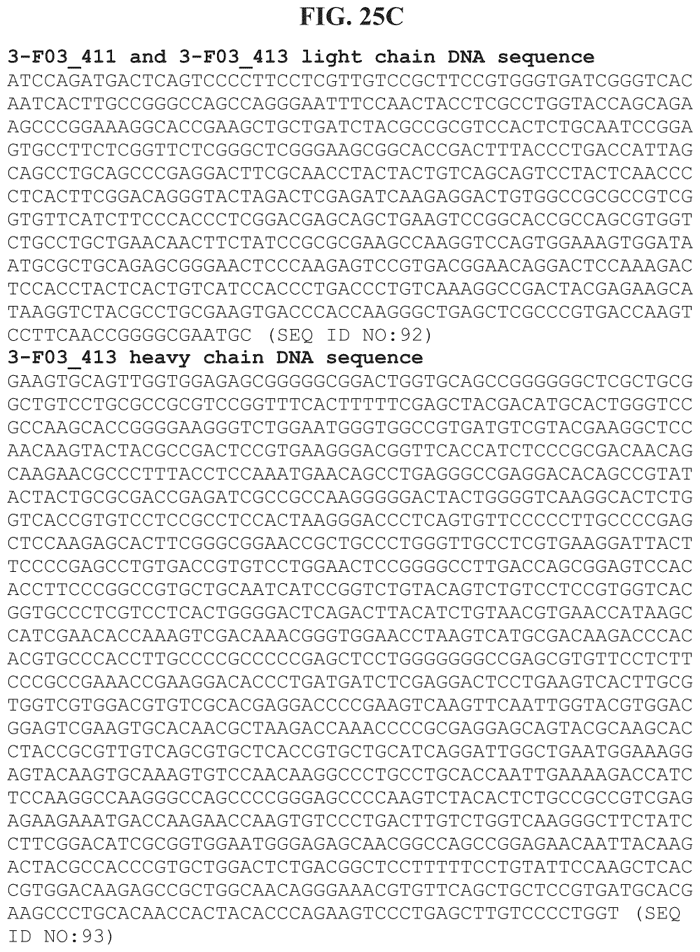

To test the ability of 3-F03 variants to inhibit CD73 activity on cells, MDA-MB-231 cells were washed with serum free RPMI media and plated 1×10⁴ cells/well in 96-well plates. Cells were incubated with the indicated concentration of antibodies or APCP at 37° C. 5% CO₂ for 30 minutes. Next, AMP was added to a final concentration of 100 μM and cells were incubated an additional 3 hours at 37° C. 5% CO₂. Plates were centrifuged for 1-2 minutes at 300 g and 25 μL of supernatant was transferred into a new 96-well plates. AMP-Glo Assay was used according to the manufacturer's instructions. RLU is a directly correlated with the AMP concentration in this assay. 3-F03 showed maximum inhibition among the 3-F03 variants (FIG. 23). Variants 3-F03_417, 3-F03_411 and 3-F03_413 displayed slightly lower potency compared to 3-F03 (FIG. 23). Variant 3-F03_412 did not inhibit membrane bound CD73 on MDA-MB-231 cells (FIG. 23).

### Example 23: Tumor Adenosine Levels of Anti-CD73 Antibody-Treated Tumors

The ability of HzCL25 and 3-F03 to modulate intra-tumoral adenosine was assessed in vivo. Female human CD34⁺ reconstituted mice (29 weeks of age; The Jackson Laboratory, Bar Harbor, ME) were inoculated subcutaneously with 3×10⁶ MDA-MB-231 cells (ATCC #HTB-26), suspended in matrigel (Corning Life Sciences) on their left flank, with three mice per treatment group. The treatment of tumor-bearing mice was started 10 days post inoculation, when tumor volume reached approximately 240 mm³. HzCL25, 3-F03 or an IgG isotype control was diluted to 1 mg/ml in phosphate-buffered saline (PBS) and administered to mice by intraperitoneal injection at a dose of 10 mg/kg on day 12, and again, 5 days later on day 17. On day 18, mice were euthanized, tumors excised, placed into cryo-tubes and flash frozen in liquid nitrogen.

To determine adenosine concentration in the tumors, frozen tissue samples were sectioned and subjected to quantitative MALDI mass spectrometry imaging (MSI) in triplicate at 80 μm spatial resolution. Based on the MSI datasets acquired from the tissue sections and a dilution series of standards spotted onto control sections, the quantification of adenosine in selected tissue sections was obtained with multi-imaging software. A correlation between the calibration curve and the signal obtained was made to determine the concentration of analytes in each region of interest. Tumor containing regions of interest were identified by H&E staining of serial sections.

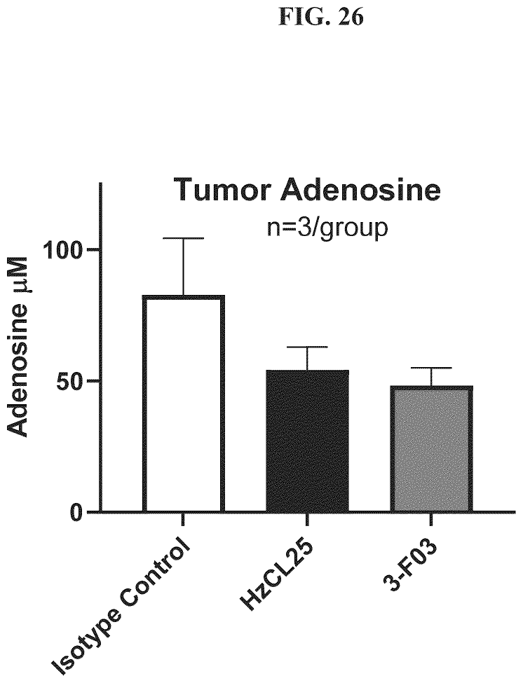

As demonstrated in FIG. 26, treatment with HzCL25 or 3-F03 resulted in a 35% and 42% decrease in intratumoral adenosine concentrations, respectively.

### Example 24: CD73 Enzyme Inhibition by Anti-CD73 Antibodies

The molecular mechanism of inhibition of 3-F03 and HzCL25 was investigated using an enzymatic assay where recombinant full length CD73 was used and the production of adenosine was measured by LCMS. The data was analyzed using a mixed-inhibition model and the findings for both 3-F03 and HzCL25 are summarized in Table 9.

Briefly, the inhibition potencies of the agents were measured in an enzyme assay using adenosine ¹³C₁₀, ¹⁵N₅ 5′-monophosphate sodium salt solution (650676, Sigma) as substrate and detecting product, adenosine by LC-MS. Antibody agents were serially diluted in buffer and a volume of 5 μL was transferred to a 96-well plate. 20 μL of 0.5 μM recombinant CD73 in assay buffer (25 mM Tris, 5 mM MgCl₂, 0.005% Brij35 pH 7.5) was added to the plate and pre-incubated for 2 hours at 25° C. The assay was initiated by the addition of a 25 μL solution containing AMP at various concentrations. The reactions were run for 0.5 hr at 25° C. and then quenched with 50 μL of methanol.

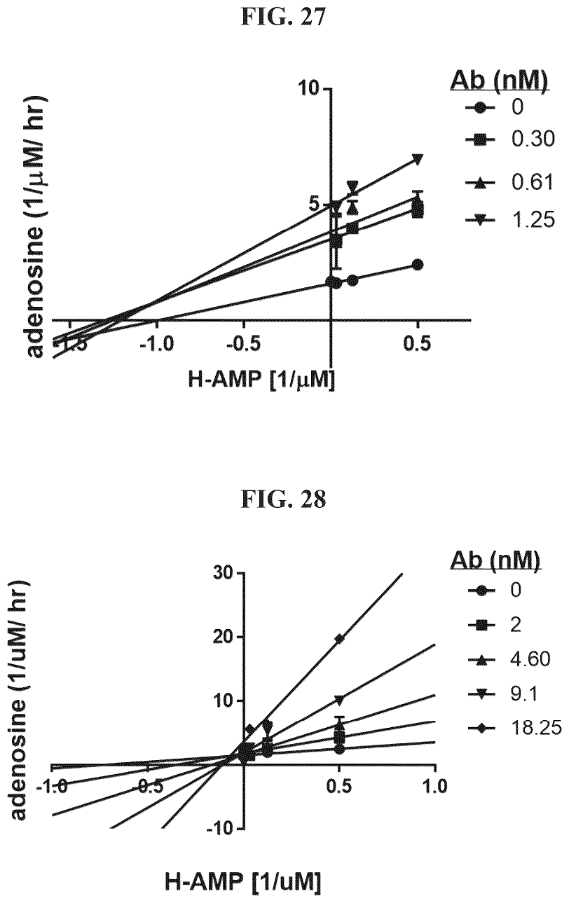

The results indicated that HzCL25 is a non-competitive inhibitor, with Ki value of 0.3±0.2 nM, and α value close to unity (FIG. 27). 3-F03 is a mixed-inhibitor with Ki of 1.5±0.7 nM and α of 19±12 indicating that 3-F03 has a higher affinity towards free enzyme than enzyme substrate, and exhibits a competitiveness nature (FIG. 28).

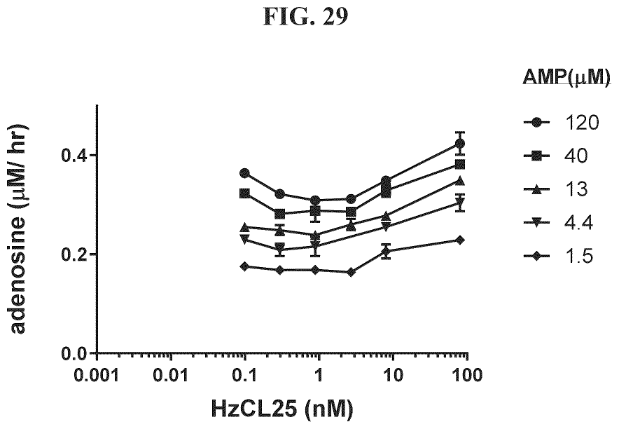

In addition, HzCL25 exhibited a “hook” effect, where a loss of inhibition was observed at high agent concentrations (FIG. 29). This is an indication that the stoichiometry of HzCL25 and CD73 molecular interactions changes as their concentration ratio changes, which shifts the molecule population from the inhibited species to non-inhibited species.

TABLE 9
Summary of kinetic parameters for HzCL25 and 3-F03.

| Agent | Mechanism of Action | Kᵢ (nM) | α | Kₘ (uM) |
|---|---|---|---|---|
| 3-F03 IgG | mixed inhibition | 1.5 ± 0.7 | 19 ± 12 | 1.4 ± 0.5 |
| HzCL25 IgG | non-competitive inhibition | 0.3 ± 0.2 | 2.1 ± 1.7 | 0.9 ± 0.3 |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody that binds to human CD73, wherein the antibody comprises a variable heavy (VH) domain comprising VH complementarity determining region (CDR)1, VH CDR2, and VH CDR3, wherein:
the VH CDR1 comprises the amino acid sequence GYTFTSYG (SEQ ID NO: 1);
the VH CDR2 comprises the amino acid sequence IYPGSGNT (SEQ ID NO:2); and
the VH CDR3 comprises the amino acid sequence ARYDYLGSSYGFDY (SEQ ID NO:3); and
wherein the antibody comprises a variable light (VL) domain comprising VL CDR1, VL CDR2, and VL CDR3, wherein:
the VL CDR1 comprises the amino acid sequence QDVSTA (SEQ ID NO:4);
the VL CDR2 comprises the amino acid sequence SAS (SEQ ID NO:5); and
the VL CDR3 comprises the amino acid sequence QQHYNTPYT (SEQ ID NO:6).

2.-12. (canceled)

13. A nucleic acid or nucleic acids encoding the antibody of claim 1.

14. An expression vector or expression vectors comprising the nucleic acid or nucleic acids of claim 13 operably linked to a promoter.

15. An isolated cell:
(a) comprising a nucleic acid or nucleic acids encoding an antibody that binds to human CD73;
(b) comprising an expression vector or expression vectors comprising a nucleic acid or nucleic acids encoding an antibody that binds to human CD73;
(c) comprising a first expression vector comprising a first nucleic acid encoding a first polypeptide comprising a variable heavy (VH) domain of an antibody that binds to human CD73 operably linked to a promoter, and a second expression vector comprising a second nucleic acid encoding a second polypeptide comprising a variable light (VL) domain of the antibody that binds to human CD73 operably linked to a promoter; or
(d) that produces an antibody that binds to human CD73;
wherein the antibody that binds to human CD73 comprises a VH domain comprising VH complementarity determining region (CDR)1, VH CDR2, and VH CDR3, wherein:
the VH CDR1 comprises the amino acid sequence GYTFTSYG (SEQ ID NO:1);
the VH CDR2 comprises the amino acid sequence IYPGSGNT (SEQ ID NO:2); and
the VH CDR3 comprises the amino acid sequence ARYDYLGSSYGFDY (SEQ ID NO:3); and
wherein the antibody comprises a VL domain comprising VL CDR1, VL CDR2, and VL CDR3, wherein:
the VL CDR1 comprises the amino acid sequence QDVSTA (SEQ ID NO:4);
the VL CDR2 comprises the amino acid sequence SAS (SEQ ID NO:5); and
the VL CDR3 comprises the amino acid sequence QQHYNTPYT (SEQ ID NO:6).

16. (canceled)

17. (canceled)

18. A method of making the antibody of claim 1, comprising culturing (i) a cell comprising a nucleic acid or nucleic acids encoding the antibody or an expression vector or expression vectors comprising a nucleic acid or nucleic acids encoding the antibody, or (ii) a cell comprising a first expression vector comprising a first nucleic acid encoding a first polypeptide comprising the VH domain of the antibody operably linked to a promoter, and a second expression vector comprising a second nucleic acid encoding a second polypeptide comprising the VL domain of the antibody operably linked to a promoter;
and isolating the antibody.

19. (canceled)

20. A method for treating a cancer in a human subject in need thereof, comprising administering to the human subject an effective amount of the antibody of claim 1.

21. (canceled)

22. (canceled)

23. An antibody that binds to human CD73, wherein the antibody comprises a variable heavy (VH) domain comprising VH complementarity determining region (CDR)1, VH CDR2, and VH CDR3, wherein:
the VH CDR1 comprises the amino acid sequence GFTFSSYD (SEQ ID NO:34);
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
wherein the antibody comprises a variable light (VL) domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39).

24.-44. (canceled)

45. A nucleic acid or nucleic acids encoding the antibody thereof of claim 23.

46. An expression vector or expression vectors comprising the nucleic acid or nucleic acids of claim 45 operably linked to a promoter.

47. An isolated cell:
(a) comprising a nucleic acid or nucleic acids encoding an antibody that binds to human CD73;
(b) comprising an expression vector or expression vectors comprising a nucleic acid or nucleic acids encoding an antibody that binds to human CD73;
(c) comprising a first expression vector comprising a first nucleic acid encoding a first polypeptide comprising a variable heavy (VH) domain of an antibody that binds to human CD73 operably linked to a promoter, and a second expression vector comprising a second nucleic acid encoding a second polypeptide comprising a variable light (VL) domain of the antibody that binds to human CD73 operably linked to a promoter; or
(d) that produces an antibody that binds to human CD73;
wherein the antibody that binds to human CD73 comprises a VH domain comprising VH complementarity determining region (CDR)1, VH CDR2, and VH CDR3, wherein:
the VH CDR1 comprises the amino acid sequence GFTFSSYD (SEQ ID NO:34):
the VH CDR2 comprises the amino acid sequence MSYDGSNK (SEQ ID NO:35) or MSYEGSNK (SEQ ID NO:40); and
the VH CDR3 comprises the amino acid sequence ATEIAAKGDY (SEQ ID NO:36); and
wherein the antibody comprises a VL domain comprising VL CDR1, VL CDR2, and VL CDR3, wherein:
the VL CDR1 comprises the amino acid sequence QGISNY (SEQ ID NO:37);
the VL CDR2 comprises the amino acid sequence AAS (SEQ ID NO:38); and
the VL CDR3 comprises the amino acid sequence QQSYSTPH (SEQ ID NO:39).

48. (canceled)

49. (canceled)

50. A method of making the antibody of claim 23, comprising culturing (i) a cell comprising a nucleic acid or nucleic acids encoding the antibody or an expression vector or expression vectors comprising a nucleic acid or nucleic acids encoding the antibody, or (ii) a cell comprising a first expression vector comprising a first nucleic acid encoding a first polypeptide comprising the VH domain of the antibody operably linked to a promoter, and a second expression vector comprising a second nucleic acid encoding a second polypeptide comprising the VL domain of the antibody operably linked to a promoter;
and isolating the antibody.

51. A pharmaceutical composition comprising the antibody of claim 23 and a pharmaceutically acceptable carrier.

52. A method for treating a cancer in a human subject in need thereof, comprising administering to the human subject an effective amount of the antibody of claim 23.

53. (canceled)

54. (canceled)


SEQUENCE LISTING (93 sequences)

SEQ ID NO: 1 - protein, 8 aa, synthetic construct
GYTFTSYG

SEQ ID NO: 2 - protein, 8 aa, synthetic construct
IYPGSGNT

SEQ ID NO: 3 - protein, 14 aa, synthetic construct
ARYDYLGSSYGFDY

SEQ ID NO: 4 - protein, 6 aa, synthetic construct
QDVSTA

SEQ ID NO: 5 - skipped

SEQ ID NO: 6 - protein, 9 aa, synthetic construct
QQHYNTPYT

SEQ ID NO: 7 - protein, 7 aa, synthetic construct
GYTFTSY

SEQ ID NO: 8 - protein, 6 aa, synthetic construct
YPGSGN

SEQ ID NO: 9 - protein, 12 aa, synthetic construct
YDYLGSSYGFDY

SEQ ID NO: 10 - protein, 11 aa, synthetic construct
KASQDVSTAVA

SEQ ID NO: 11 - protein, 7 aa, synthetic construct
SASYRYS

SEQ ID NO: 12 - protein, 10 aa, synthetic construct
GYTFTSYGLS

SEQ ID NO: 13 - protein, 10 aa, synthetic construct
EIYPGSGNTY

SEQ ID NO: 14 - protein, 5 aa, synthetic construct
SYGLS

SEQ ID NO: 15 - protein, 17 aa, synthetic construct
EIYPGSGNTYYNEKFKG

SEQ ID NO: 16 - protein, 6 aa, synthetic construct
TSYGLS

SEQ ID NO: 17 - protein, 13 aa, synthetic construct
WMGEIYPGSGNTY

SEQ ID NO: 18 - protein, 13 aa, synthetic construct
ARYDYLGSSYGFD

SEQ ID NO: 19 - protein, 7 aa, synthetic construct
STAVAWY

SEQ ID NO: 20 - protein, 10 aa, synthetic construct
LLIYSASYRY

SEQ ID NO: 21 - protein, 8 aa, synthetic construct
QQHYNTPY

SEQ ID NO: 22 - protein, 121 aa, synthetic construct
EVQLVQSGAEVKKPGESLKISCKGSGYTFTSYGLSWVRQMPGKGLEWMGEIYPGSGNTYYNEKFKGQVTISADKSISTAYLQWSSLKASDTAMYYCARYDYLGSSYGFDYWGAGTTVTVSS

SEQ ID NO: 23 - protein, 107 aa, synthetic construct
DIVMTQSPDSLAVSLGERATINCKASQDVSTAVAWYQQKPGQPPKLLIYSASYRYSGVPDRFSGSGSGTDFTLTISSLQAEDVAVYYCQQHYNTPYTFGGGTKLEIK

SEQ ID NO: 24 - protein, 450 aa, synthetic construct
EVQLVQSGAEVKKPGESLKISCKGSGYTFTSYGLSWVRQMPGKGLEWMGEIYPGSGNTYYNEKFKGQVTISADKSISTAYLQWSSLKASDTAMYYCARYDYLGSSYGFDYWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 214 aa, synthetic construct
DIVMTQSPDSLAVSLGERATINCKASQDVSTAVAWYQQKPGQPPKLLIYSASYRYSGVPDRFSGSGSGTDFTLTISSLQAEDVAVYYCQQHYNTPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 121 aa, synthetic construct
QVQLQQSGAELARPGASVKLSCRASGYTFTSYGLSWVKQRTGQGLEWIGEIYPGSGNTYYNEKFKGKATLTADKSSSTAYMELRSLTSEDSAVYFCARYDYLGSSYGFDYWGQGTTLTVSS

SEQ ID NO: 27 - protein, 107 aa, synthetic construct
DIVMTQSHKFMSTSVGDRVSITCKASQDVSTAVAWYQQKPGQSPKLLIYSASYRYNGVPDRFTGSGSGTDFTFTISSVQAEDLAVYYCQQHYNTPYTFGGGTKLEIK

SEQ ID NO: 28 - protein, 13 aa, synthetic construct
WIGEIYPGSGNTY

SEQ ID NO: 29 - protein, 7 aa, synthetic construct
SASYRYN

SEQ ID NO: 30 - protein, 446 aa, synthetic construct
EVQLVESGGGLVQPGGSLRLSCAASGFTFSSYDMHWVRQAPGKGLEWVAVMSYDGSNKYYADSVKGRFTISRDNSKNALYLQMNSLRAEDTAVYYCATEIAAKGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 212 aa, synthetic construct
IQMTQSPSSLSASVGDRVTITCRASQGISNYLAWYQQKPGKAPKLLIYAASTLQSGVPSRFSGSGSGTDFTLTISSLQPEDFATYYCQQSYSTPHFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 116 aa, synthetic construct
VQLVESGGGLVQPGGSLRLSCAASGFTFSSYDMHWVRQAPGKGLEWVAVMSYSGSNKYYADSVKGRFTISRDNSKNTLYLQMNSLRAEDTAVYYCATEIAAKGDYWGQGTLVTVSS

SEQ ID NO: 33 - protein, 446 aa, synthetic construct
EVQLVESGGGLVQPGGSLRLSCAASGFTFSSYDMHWVRQAPGKGLEWVAVMSYEGSNKYYADSVKGRFTISRDNSKNALYLQMNSLRAEDTAVYYCATEIAAKGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 8 aa, synthetic construct
GFTFSSYD

SEQ ID NO: 35 - protein, 8 aa, synthetic construct
MSYDGSNK

SEQ ID NO: 36 - protein, 10 aa, synthetic construct
ATEIAAKGDY

SEQ ID NO: 37 - protein, 6 aa, synthetic construct
QGISNY

SEQ ID NO: 38 - skipped

SEQ ID NO: 39 - protein, 8 aa, synthetic construct
QQSYSTPH

SEQ ID NO: 40 - protein, 8 aa, synthetic construct
MSYEGSNK

SEQ ID NO: 41 - protein, 7 aa, synthetic construct
GFTFSSY

SEQ ID NO: 42 - protein, 6 aa, synthetic construct
SYDGSN

SEQ ID NO: 43 - protein, 6 aa, synthetic construct
SYEGSN

SEQ ID NO: 44 - protein, 11 aa, synthetic construct
RASQGISNYLA

SEQ ID NO: 45 - protein, 7 aa, synthetic construct
AASTLQS

SEQ ID NO: 46 - protein, 10 aa, synthetic construct
GFTFSSYDMH

SEQ ID NO: 47 - protein, 10 aa, synthetic construct
VMSYDGSNKY

SEQ ID NO: 48 - protein, 10 aa, synthetic construct
VMSYEGSNKY

SEQ ID NO: 49 - protein, 5 aa, synthetic construct
SYDMH

SEQ ID NO: 50 - protein, 17 aa, synthetic construct
VMSYDGSNKYYADSVKG

SEQ ID NO: 51 - protein, 17 aa, synthetic construct
VMSYEGSNKYYADSVKG

SEQ ID NO: 52 - protein, 8 aa, synthetic construct
EIAAKGDY

SEQ ID NO: 53 - protein, 6 aa, synthetic construct
SSYDMH

SEQ ID NO: 54 - protein, 13 aa, synthetic construct
WVAVMSYDGSNKY

SEQ ID NO: 55 - protein, 13 aa, synthetic construct
WVAVMSYEGSNKY

SEQ ID NO: 56 - protein, 9 aa, synthetic construct
ATEIAAKGD

SEQ ID NO: 57 - protein, 7 aa, synthetic construct
SNYLAWY

SEQ ID NO: 58 - protein, 10 aa, synthetic construct
LLIYAASTLQ

SEQ ID NO: 59 - protein, 7 aa, synthetic construct
QQSYSTP

SEQ ID NO: 60 - protein, 116 aa, synthetic construct
VQLVESGGGLVQPGGSLRLSCAASGFTFSSYDMHWVRQAPGKGLEWVAVMSYDGSNKYYADSVKGRFTISRDNSKNALYLQMNSLRAEDTAVYYCATEIAAKGDYWGQGTLVTVSS

SEQ ID NO: 61 - protein, 105 aa, synthetic construct
IQMTQSPSSLSASVGDRVTITCRASQGISNYLAWYQQKPGKAPKLLIYAASTLQSGVPSRFSGSGSGTDFTLTISSLQPEDFATYYCQQSYSTPHFGQGTRLEIK

SEQ ID NO: 62 - protein, 117 aa, synthetic construct
EVQLVESGGGLVQPGGSLRLSCAASGFTFSSYDMHWVRQAPGKGLEWVAVMSYDGSNKYYADSVKGRFTISRDNSKNALYLQMNSLRAEDTAVYYCATEIAAKGDYWGQGTLVTVSS

SEQ ID NO: 63 - protein, 117 aa, synthetic construct
EVQLVESGGGLVQPGGSLRLSCAASGFTFSSYDMHWVRQAPGKGLEWVAVMSYEGSNKYYADSVKGRFTISRDNSKNALYLQMNSLRAEDTAVYYCATEIAAKGDYWGQGTLVTVSS

SEQ ID NO: 64 - protein, 106 aa, synthetic construct
DIQMTQSPSSLSASVGDRVTITCRASQGISNYLAWYQQKPGKAPKLLIYAASTLQSGVPSRFSGSGSGTDFTLTISSLQPEDFATYYCQQSYSTPHFGQGTRLEIK

SEQ ID NO: 65 - protein, 106 aa, synthetic construct
AIQMTQSPSSLSASVGDRVTITCRASQGISNYLAWYQQKPGKAPKLLIYAASTLQSGVPSRFSGSGSGTDFTLTISSLQPEDFATYYCQQSYSTPHFGQGTRLEIK

SEQ ID NO: 66 - protein, 446 aa, synthetic construct
VQLVESGGGLVQPGGSLRLSCAASGFTFSSYDMHWVRQAPGKGLEWVAVMSYDGSNKYYADSVKGRFTISRDNSKNALYLQMNSLRAEDTAVYYCATEIAAKGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 116 aa, synthetic construct
VQLVESGGGLVQPGGSLRLSCAASGFTFSSYDMHWVRQAPGKGLEWVAVMSYSGSNKYYADSVKGRFTISRDNSKNALYLQMNSLRAEDTAVYYCATEIAAKGDYWGQGTLVTVSS

SEQ ID NO: 68 - protein, 117 aa, synthetic construct
EVQLVESGGGLVQPGGSLRLSCAASGFTFSSYDMHWVRQAPGKGLEWVAVMSYSGSNKYYADSVKGRFTISRDNSKNTLYLQMNSLRAEDTAVYYCATEIAAKGDYWGQGTLVTVSS

SEQ ID NO: 69 - protein, 117 aa, synthetic construct
EVQLVESGGGLVQPGGSLRLSCAASGFTFSSYDMHWVRQAPGKGLEWVAVMSYSGSNKYYADSVKGRFTISRDNSKNALYLQMNSLRAEDTAVYYCATEIAAKGDYWGQGTLVTVSS

SEQ ID NO: 70 - protein, 523 aa, Homo sapiens
WELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFS

SEQ ID NO: 71 - protein, 523 aa, Mus musculus
WELTILHTNDVHSRLEQTSDDSTKCLNASLCVGGVARLFTKVQQIRKEEPNVLFLDAGDQYQGTIWFTVYKGLEVAHFMNILGYDAMALGNHEFDNGVEGLIDPLLRNVKFPILSANIKARGPLAHQISGLFLPSKVLSVGGEVVGIVGYTSKETPFLSNPGTNLVFEDEISALQPEVDKLKTLNVNKIIALGHSGFEMDKLIAQKVRGVDIVVGGHSNTFLYTGNPPSKEVPAGKYPFIVTADDGRQVPVVQAYAFGKYLGYLKVEFDDKGNVITSYGNPILLNSSIPEDATIKADINQWRIKLDNYSTQELGRTIVYLDGSTQTCRFRECNMGNLICDAMINNNLRHPDEMFWNHVSMCIVNGGGIRSPIDEKNNGTITWENLAAVLPFGGTFDLVQLKGSTLKKAFEHSVHRYGQSTGEFLQVGGIHVVYDINRKPWNRVVQLEVLCTKCRVPIYEPLEMDKVYKVTLPSYLANGGDGFQMIKDELLKHDSGDQDISVVSEYISKMKVVYPAVEGRIKFS

SEQ ID NO: 72 - protein, 523 aa, Macaca fascicularis
WELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFETDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHADEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEIYKVILPNFLANGGDGFQMIKDELLRHDSGDQDINVVSTYISKMKVIYPAVEGRIKFS

SEQ ID NO: 73 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 107 aa, Homo sapiens [sequence omitted]

SEQ ID NO: 75 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 14 aa, Homo sapiens
TKVQQIRRAEPNVL

SEQ ID NO: 77 - protein, 117 aa, synthetic construct
EVQLVESGGGLVQPGGSLRLSCAASGFTFSSYDMHWVRQAPGKGLEWVAVMSYDGSNKYYADSVKGRFTISRDNSKNTLYLQMNSLRAEDTAVYYCATEIAAKGDYWGQGTLVTVSS

SEQ ID NO: 78 - protein, 14 aa, Homo sapiens
AAVLPFGGTFDLVQ

SEQ ID NO: 79 - protein, 20 aa, Homo sapiens
ILPNFLANGGDGFQMIKDEL

SEQ ID NO: 80 - protein, 107 aa, synthetic construct
DIQMTQSPSSLSASVGDRVTITCKASQDVSTAVAWYQQKPGKAPKLLLYSASYRYSGVPSRFSGSGSGTDYTLTISSLQPEDFATYYCQQHYNTPYTFGGGTKLEIK

SEQ ID NO: 81 - protein, 107 aa, synthetic construct
AIRMTQSPSSFSASTGDRVTITCKASQDVSTAVAWYQQKPGKAPKLLIYSASYRYSGVPSRFSGSGSGTDFTLTISCLQSEDFATYYCQQHYNTPYTFGGGTKLEIK

SEQ ID NO: 82 - protein, 121 aa, synthetic construct
QVQLVQSGAEVKKPGASVKVSCKASGYTFTSYGLSWVRQATGQGLEWMGEIYPGSGNTYYNEKFKGRVTMTANTSISTAYMELSSLRSEDTAVYYCARYDYLGSSYGFDYWGAGTTVTVSS

SEQ ID NO: 83 - protein, 121 aa, synthetic construct
QVQLVQSGAEVKKPGASVKVSCKASGYTFTSYGLSWVRQATGQGLEWMGEIYPGSGNTYYNEKFKGRVTMTRNTSISTAYMELSSLRSEDTAVYYCARYDYLGSSYGFDYWGAGTTVTVSS

SEQ ID NO: 84 - protein, 121 aa, synthetic construct
QVQLVQSGAEVKKPGSSVKVSCKASGYTFTSYGLSWVRQAPGQGLEWMGEIYPGSGNTYYNEKFKGRVTITADESTSTAYMELSSLRSEDTAVYYCARYDYLGSSYGFDYWGAGTTVTVSS

SEQ ID NO: 85 - protein, 116 aa, synthetic construct
VQLVESGGGLVQPGGSLRLSCAASGFTFSSYDMHWVRQAPGKGLEWVAVMSYDGSNKYYADSVKGRFTISRDNSKNTLYLQMNSLRAEDTAVYYCATEIAAKGDYWGQGTLVTVSS

SEQ ID NO: 86 - protein, 117 aa, synthetic construct
EVQLVESGGGLVQPGGSLRLSCAASGFTFSSYDMHWVRQAPGKGLEWVAVMSYEGSNKYYADSVKGRFTISRDNSKNTLYLQMNSLRAEDTAVYYCATEIAAKGDYWGQGTLVTVSS

SEQ ID NO: 87 - protein, 116 aa, synthetic construct
VQLVESGGGLVQPGGSLRLSCAASGFTFSSYDMHWVRQAPGKGLEWVAVMSYEGSNKYYADSVKGRFTISRDNSKNTLYLQMNSLRAEDTAVYYCATEIAAKGDYWGQGTLVTVSS

SEQ ID NO: 88 - protein, 116 aa, synthetic construct
VQLVESGGGLVQPGGSLRLSCAASGFTFSSYDMHWVRQAPGKGLEWVAVMSYEGSNKYYADSVKGRFTISRDNSKNALYLQMNSLRAEDTAVYYCATEIAAKGDYWGQGTLVTVSS

SEQ ID NO: 89 - DNA, 1350 bp, synthetic construct
  source 1..1350: mol_type = other DNA
gaagtgcagctcgtgcagtccggagccgaagtgaaaaagcctggagagtccctgaagatcagctgcaagggttccggctatacattcacctcctacgggctcagctgggtcagacagatgccgggaaagggtcttgagtggatgggagagatctacccgggctccggcaacacctactacaacgaaaagttcaagggccaggtcaccatttccgccgacaagtcaatctccaccgcttacctccaatggtcgagcctgaaggcatcggataccgcgatgtactactgcgcccgctacgactacctgggctcgtcatacggcttcgattactggggggcgggaactaccgtgactgtgtcctccgcctccactaagggaccctcagtgttcccccttgccccgagctccaagagcacttcgggcggaaccgctgccctgggttgcctcgtgaaggattacttccccgagcctgtgaccgtgtcctggaactccggggccttgaccagcggagtccacaccttcccggccgtgctgcaatcatccggtctgtacagtctgtcctccgtggtcacggtgccctcgtcctcactggggactcagacttacatctgtaacgtgaaccataagccatcgaacaccaaagtcgacaaacgggtggaacctaagtcatgcgacaagacccacacgtgcccaccttgccccgcccccgagctcctgggggggccgagcgtgttcctcttcccgccgaaaccgaaggacaccctgatgatctcgaggactcctgaagtcacttgcgtggtcgtggacgtgtcgcacgaggaccccgaagtcaagttcaattggtacgtggacggagtcgaagtgcacaacgctaagaccaaaccccgcgaggagcagtacgcaagcacctaccgcgttgtcagcgtgctcaccgtgctgcatcaggattggctgaatggaaaggagtacaagtgcaaagtgtccaacaaggccctgcctgcaccaattgaaaagaccatctccaaggccaagggccagccccgggagccccaagtctacactctgccgccgtcgagagaagaaatgaccaagaaccaagtgtccctgacttgtctggtcaagggcttctatccttcggacatcgcggtggaatgggagagcaacggccagccggagaacaattacaagactacgccacccgtgctggactctgacggctcctttttcctgtattccaagctcaccgtggacaagagccgctggcaacagggaaacgtgttcagctgctccgtgatgcacgaagccctgcacaaccactacacccagaagtccctgagcttgtcccctggt

SEQ ID NO: 90 - DNA, 642 bp, synthetic construct
  source 1..642: mol_type = other DNA
gacatcgtgatgacccagtccccggattcactcgcggtgtctttgggggagagggcaaccattaactgcaaggcctcacaggatgtgtccactgctgtcgcctggtaccagcagaagcctgggcagccgcccaagctgctgatctactcggcctcctaccgctattccggagtccccgaccggttctccggctcgggttccggaactgatttcaccctgacaatttcgtcgctgcaagccgaggacgtggccgtgtactactgccaacagcattacaacactccttacacttttggtggcggaactaagctcgagatcaagcggacggtggcagctccgtcagtgttcatcttccctccatcggacgaacagctgaagtccggcaccgcgtccgtcgtgtgtctgttgaacaacttctacccgcgggaagccaaggtccagtggaaagtcgacaacgcgctgcagtccggaaatagccaggaaagcgtgaccgaacaggactccaaggacagcacctactccctgagctcaaccctgaccctgagcaaggccgactatgagaagcacaaagtgtacgcctgcgaagtgacccaccaaggcctgagcagcccagtgaccaagtccttcaaccgcggggagtgt

SEQ ID NO: 91 - DNA, 1338 bp, synthetic construct
  source 1..1338: mol_type = other DNA
gaagtgcagttggtggagagcgggggcggactggtgcagccggggggctcgctgcggctgtcctgcgccgcgtccggtttcactttttcgagctacgacatgcactgggtccgccaagcaccggggaagggtctggaatgggtggccgtgatgtcgtacgacggctccaacaagtactacgccgactccgtgaagggacggttcaccatctcccgcgacaacagcaagaacgccctttacctccaaatgaacagcctgagggccgaggacacagccgtatactactgcgcgaccgagatcgccgccaagggggactactggggtcaaggcactctggtcaccgtgtcctccgcctccactaagggaccctcagtgttcccccttgccccgagctccaagagcacttcgggcggaaccgctgccctgggttgcctcgtgaaggattacttccccgagcctgtgaccgtgtcctggaactccggggccttgaccagcggagtccacaccttcccggccgtgctgcaatcatccggtctgtacagtctgtcctccgtggtcacggtgccctcgtcctcactggggactcagacttacatctgtaacgtgaaccataagccatcgaacaccaaagtcgacaaacgggtggaacctaagtcatgcgacaagacccacacgtgcccaccttgccccgcccccgagctcctgggggggccgagcgtgttcctcttcccgccgaaaccgaaggacaccctgatgatctcgaggactcctgaagtcacttgcgtggtcgtggacgtgtcgcacgaggaccccgaagtcaagttcaattggtacgtggacggagtcgaagtgcacaacgctaagaccaaaccccgcgaggagcagtacgcaagcacctaccgcgttgtcagcgtgctcaccgtgctgcatcaggattggctgaatggaaaggagtacaagtgcaaagtgtccaacaaggccctgcctgcaccaattgaaaagaccatctccaaggccaagggccagccccgggagccccaagtctacactctgccgccgtcgagagaagaaatgaccaagaaccaagtgtccctgacttgtctggtcaagggcttctatccttcggacatcgcggtggaatgggagagcaacggccagccggagaacaattacaagactacgccacccgtgctggactctgacggctcctttttcctgtattccaagctcaccgtggacaagagccgctggcaacagggaaacgtgttcagctgctccgtgatgcacgaagccctgcacaaccactacacccagaagtccctgagcttgtcccctggt

SEQ ID NO: 92 - DNA, 636 bp, synthetic construct
  source 1..636: mol_type = other DNA
atccagatgactcagtccccttcctcgttgtccgcttccgtgggtgatcgggtcacaatcacttgccgggccagccagggaatttccaactacctcgcctggtaccagcagaagcccggaaaggcaccgaagctgctgatctacgccgcgtccactctgcaatccggagtgccttctcggttctcgggctcgggaagcggcaccgactttaccctgaccattagcagcctgcagcccgaggacttcgcaacctactactgtcagcagtcctactcaacccctcacttcggacagggtactagactcgagatcaagaggactgtggccgcgccgtcggtgttcatcttcccaccctcggacgagcagctgaagtccggcaccgccagcgtggtctgcctgctgaacaacttctatccgcgcgaagccaaggtccagtggaaagtggataatgcgctgcagagcgggaactcccaagagtccgtgacggaacaggactccaaagactccacctactcactgtcatccaccctgaccctgtcaaaggccgactacgagaagcataaggtctacgcctgcgaagtgacccaccaagggctgagctcgcccgtgaccaagtccttcaaccggggcgaatgc

SEQ ID NO: 93 - DNA, 1338 bp, synthetic construct
  source 1..1338: mol_type = other DNA
gaagtgcagttggtggagagcgggggcggactggtgcagccggggggctcgctgcggctgtcctgcgccgcgtccggtttcactttttcgagctacgacatgcactgggtccgccaagcaccggggaagggtctggaatgggtggccgtgatgtcgtacgaaggctccaacaagtactacgccgactccgtgaagggacggttcaccatctcccgcgacaacagcaagaacgccctttacctccaaatgaacagcctgagggccgaggacacagccgtatactactgcgcgaccgagatcgccgccaagggggactactggggtcaaggcactctggtcaccgtgtcctccgcctccactaagggaccctcagtgttcccccttgccccgagctccaagagcacttcgggcggaaccgctgccctgggttgcctcgtgaaggattacttccccgagcctgtgaccgtgtcctggaactccggggccttgaccagcggagtccacaccttcccggccgtgctgcaatcatccggtctgtacagtctgtcctccgtggtcacggtgccctcgtcctcactggggactcagacttacatctgtaacgtgaaccataagccatcgaacaccaaagtcgacaaacgggtggaacctaagtcatgcgacaagacccacacgtgcccaccttgccccgcccccgagctcctgggggggccgagcgtgttcctcttcccgccgaaaccgaaggacaccctgatgatctcgaggactcctgaagtcacttgcgtggtcgtggacgtgtcgcacgaggaccccgaagtcaagttcaattggtacgtggacggagtcgaagtgcacaacgctaagaccaaaccccgcgaggagcagtacgcaagcacctaccgcgttgtcagcgtgctcaccgtgctgcatcaggattggctgaatggaaaggagtacaagtgcaaagtgtccaacaaggccctgcctgcaccaattgaaaagaccatctccaaggccaagggccagccccgggagccccaagtctacactctgccgccgtcgagagaagaaatgaccaagaaccaagtgtccctgacttgtctggtcaagggcttctatccttcggacatcgcggtggaatgggagagcaacggccagccggagaacaattacaagactacgccacccgtgctggactctgacggctcctttttcctgtattccaagctcaccgtggacaagagccgctggcaacagggaaacgtgttcagctgctccgtgatgcacgaagccctgcacaaccactacacccagaagtccctgagcttgtcccctggt